SEQUENCE LISTING (24 sequences)

SEQ ID NO: 1 - protein, 422 aa, Maraba virus
MSVTVKRVIDDSLITPKLPANEDPVEYPADYFKKSRDIPVYINTTKSLSDLRGYVYQGLKSGNISIIHVNSYLYAALKEIRGKLDRDWITFGIQIGKTGDSVGIFDLLTLKPLDGVLPDGVSDATRTSSDDAWLPLYLLGLYRVGRTQMPEYRKKLMDGLINQCKMINEQFEPLLPEGRDVFDVWGNDSNYTKIVAAVDMFFHMFKKHEKASFRYGTIVSRFKDCAALATFGHLCKITGMSTEDVTTWILNREVADEMVQMMYPGQEIDKADSYMPYLIDLGLSSKSPYSSVKNPAFHFWGQLTALLLRSTRARNARQPDDIEYTSLTTAGLLYAYAVGSSADLAQQFYVGDNKYVPETGDGGLTTNAPPQGRDVVEWLSWFEDQNRKPTPDMLMYAKRAVSALQGLREKTIGKYAKSEFDK

SEQ ID NO: 2 - protein, 265 aa, Maraba virus
MDQLSKVKEFLKTYAQLDQAVQEMDDIESQREEKTNFDLFQEEGLEIKEKPSYYRADEEEIDSDEDSVDDAQDLGIRTSTSPIEGYVDEEQDDYEDEEVNVVFTSDWKQPELESDGDGKTLRLTIPDGLTGEQKSQWLATIKAVVQSAKYWNISECSFESYEQGVLIRERQMTPDVYKVTPVLNAPPVQMTANQDVWSLSSTPFTFLPKKQGVTPLTMSLEELFNTRGEFISLGGNGKMSHREAIILGLRHKKLYNQARLKYNLA

SEQ ID NO: 3 - protein, 2109 aa, Maraba virus
MDVNDFELHEDFALSEDDFVTSEFLNPEDQMTYLNHADYNLNSPLISDDIDFLIKKYNHEQIPKMWDVKNWEGVLEMLTACQASPILSSTMHKWVGKWLMSDDHDASQGFSFLHEVDKEADLTFEVVETFIRGWGGRELQYKRKDTFPDSFRVAASLCQKFLDLHKLTLIMNSVSEVELTNLAKNFKGKNRKAKSGNLITRLRVPSLGPAFVTQGWVYMKKLEMIMDRNFLLMLKDVIIGRMQTILSMISRDDNLFSESDIFTVLKIYRIGDKILERQGTKGYDLIKMIEPICNLKMMNLARKYRPLIPTFPHFEKHIADSVKEGSKIDKGIEFIYDHIMSIPGVDLTLVIYGSFRHWGHPFINYYEGLEKLHKQVTMPKTIDREYAECLASDLARIVLQQQFNEHKKWFVDVDKVPQSHPFKSHMKENTWPTAAQVQDYGDRWHQLPLIKCFEIPDLLDPSIIYSDKSHSMNRSEVLRHVRLTPHVPIPSRKVLQTMLETKATDWKEFLKKIDEEGLEDDDLVIGLKGKERELKIAGRFFSLMSWKLREYFVITEYLIKTHFVPMFKGLTMADDLTAVIKKMMDTSSGQGLDNYESICIANHIDYEKWNNHQRKESNGPVFKVMGQFLGYPRLIERTHEFFEKSLIYYNGRPDLMRVRGNSLVNASSLNVCWEGQAGGLEGLRQKGWSILNLLVIQREAKIRNTAVKVLAQGDNQVICTQYKTKKSRNDIELKAALTQMVSNNEMIMSAIKSGTEKLGLLINDDETMQSADYLNYGKVPIFRGVIRGLETKRWSRVTCVTNDQIPTCANIMSSVSTNALTVAHFAENPVNAIIQYNYFGTFARLLLMMHDPALRISLYEVQSKIPGLHSLTFKYSMLYLDPSIGGVSGMSLSRFLIRSFPDPVTESLAFWKFIHSHARSDSLKEICAVFGNPEIARFRLTHVDKLVEDPTSLNIAMGMSPANLLKTEVKKCLLESRQSIKNQIVRDATIYLHHEEDKLRSFLWSITPLFPRFLSEFKSGTFIGVADGLISLFQNSRTIRNSFKKRYHRELDDLIIKSEVSSLMHLGKLHLRRGSVRMWTCSSTQADLLRFRSWGRSVIGTTVPHPLEMLGQHFKKETPCSACNISGLDYVSVHCPNGIHDVFESRGPLPAYLGSKTSESTSILQPWERESKVPLIKRATRLRDAISWFVSPDSNLASTILKNINALTGEEWSKKQHGFKRTGSALHRFSTSRMSHGGFASQSTAALTRLMATTDTMRDLGEQNYDFLFQATLLYAQITTTVVRNGSFHSCTDHYHITCKSCLRAIDEITLDSAMEYSPPDVSSVLQSWRNGEGSWGHEVKQIYPVEGDWRGLSPVEQSYQVGRCIGFLFGDLAYRKSSHADDSSMFPLSIQNKVRGRGFLKGLMDGLMRASCCQVIHRRSLAHLKRPANAVYGGLIYLIDKLSASAPFLSLTRHGPLREELETVPHKIPTSYPTSNRDMGVIVRNYFKYQCRLVEKGRYKTHYPQLWLFSDVLSIDFLGPLSISSTLLGILYKQTLSSRDKNELRELANLSSLLRSGEGWEDIHVKFFSKDTLLCPEEIRHACKFGIAKESAVLSYYPPWSQESYGGITSIPVYFSTRKYPKILDVPPRVQNPLVSGLRLGQLPTGAHYKIRSIVKNKNLRYRDFLSCGDGSGGMTAALLRENRQSRGIFNSLLELAGSLMRGASPEPPSALETLGQERSRCVNGSTCWEYSSDLSQKETWDYFLRLKRGLGLTVDLITMDMEVRDPNTSLMIEKNLKVYLHQILEPTGVLIYKTYGTHIATQTDNILTIIGPFFETVDLVQSEYSSSQTSEVYFVGRGLRSHVDEPWVDWPSLMDNWRSIYAFHDPTTEFIRAKKVCEIDSLIGIPAQFIPDPFVNLETMLQIVGVPTGVSHAAALLSSQYPNQLVTTSIFYMTLVSYYNVNHIRRSPKPFSPPSDGVSQNIGSAIVGLSFWVSLMENDLGLYKQALGAIKTSFPIRWSSVQTKDGFTQEWRTKGNGIPKDCRLSDSLAQIGNWIRAMELVRNKTRQSGFSETLFDQFCGLADHHLKWRKLGNRTGIIDWLNNRISSIDKSILVTKSDLHDENSWRE

SEQ ID NO: 4 - protein, 229 aa, Maraba virus
MSSLKKILGIKGKGKKSKKLGMAPPPYEEETPMEYSPSAPYDKSLFGVEDMDFHDQRQLRYEKFHFSLKMTVRSNKPFRNYDDVAAAVSNWDHMYIGMAGKRPFYKILAFMGSTLLKATPAVLADQGQPEYHAHCEGRAYLPHRLGPTPPMLNVPEHFRRPFNIGLFRGTIDITLVLFDDESVDSAPVIWDHFNASRLSSFREKALLFGLILEKKATGNWVLDSISHFK

SEQ ID NO: 5 - protein, 228 aa, Artificial
  mutant of Maraba protein M
MSSLKKILGIKGKGKKSKKLGMAPPPYEEETPMEYSPSAPYDKSLFGVEDDFHDQRQLRYEKFHFSLKMTVRSNKPFRNYDDVAAAVSNWDHMYIGMAGKRPFYKILAFMGSTLLKATPAVLADQGQPEYHAHCEGRAYLPHRLGPTPPMLNVPEHFRRPFNIGLFRGTIDITLVLFDDESVDSAPVIWDHFNASRLSSFREKALLFGLILEKKATGNWVLDSISHFK

SEQ ID NO: 6 - protein, 591 aa, Bahia Grande virus
MISNMFFLFQLSLFLQFIAGDESLETITAPETPDPILLKGDTKYLFLVPSSVKNWKPADLNELTCPPLISKPDTSEMTYFSTDVMELQKHHELAPVEGYLCSGLRYKVICSEGFFGQKTIAKKIENIEPDSKQCLDDLSKFKNDDYLLPYFPSEDCNWMKETPTHKDFIVFQKHFVKYDPYNNGFYDPLLKKDYCDTQVCETEHDQTIWITEKSIENECIFNYPIKKHIFHTADFGKMIIDYELNQWTSVEDGCLINYCGREGIRLSNGMFFVGKFYKNLNNLQTCSAGTKVSYKPLTSKLEEIENEIILDQERLLCLDSIRQMTATKKLSFYSLSFLEPKSSSRHKVFRIHNKTLEYTETEWHPIMSFNFDEPNKIGIDKNGKSVYWNEWVPSGISGLLSGFNGVYKKENETKVTIARLETIKEDYDREMMIDHELVEVEHPKIVHLKRENITGSRVEIVNKEHSDVSGWLSSVLSSFWGKIMMTIISIILIVIIGLVLINCCPIICKSCIKRYKTKEESRNRHRLDREDNGRLRRQHRVIFNNQSNDEENAIEMVEYTDTPRPLRPIPDATTSDTESRSPTTAHSFFNR

SEQ ID NO: 7 - protein, 498 aa, Lymphocytic choriomeningitis virus
MGQIVTMFEALPHIIDEVINIVIIVLIIITSIKAVYNFATCGILALVRFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAISQCRTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 8 - protein, 676 aa, Ebola virus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 9 - DNA, 11380 bp, Artificial
  DNA encoded by chimeric virus that contains RNA from Maraba and Bahia Grande viruses
acgaagacaaacaaaccattgatagaattaagaggctcatgaaaatccttaacagcgttcaaaatgtctgttacagtcaagagagtcattgatgattcactcatcacccccaaattgcctgcgaatgaggaccctgtggagtaccctgctgattatttcaaaaagtcccgtgatattccggtgtacataaacacgaccaaaagtttgtctgatttgcggggctatgtttatcaaggcctaaagtcaggcaacatctctataattcatgtcaacagttatctgtatgcagcattaaaagagatcagaggaaaattggacagagattggatcacctttggtatccaaatcggaaaaacaggagatagcgtggggatattcgatttactgaccctaaaacctctagatggtgttttaccagatggggtgtctgatgctactcgaactagctcagacgatgcatggcttccactgtatctattggggttatacagagttggtcgaacacagatgccagaatacaggaagaagctgatggatggtctgattaatcaatgtaagatgatcaatgagcagtttgaaccactgttgccagaaggaagagatgtctttgatgtctggggaaatgacagcaattacacaaagattgtggccgctgtagatatgttcttccatatgttcaaaaagcatgagaaggcctctttcaggtatggcacaatagtgtcaagatttaaggattgtgcagcattggctacatttggtcatctgtgtaagatcactggtatgtccactgaagatgtgacaacttggattctaaacagggaggtggctgatgagatggttcaaatgatgtacccaggacaggagatagataaggctgattcttacatgccttatctaatcgacttaggtctgtcctcaaaatctccatattcatcagttaaaaatccagctttccatttttggggtcaattgaccgcattgttactgagatcaaccagagccagaaatgcacgtcagccggatgacatcgagtatacatccctgaccactgctgggctgttgtatgcatatgccgttggttcgtctgcagacctggctcaacaattctacgttggggacaacaagtatgtgccagaaactggagatggaggattaaccaccaatgcaccgccacaagggcgagatgtggtcgagtggcttagttggtttgaagatcaaaacagaaaacctaccccagacatgctcatgtatgctaagagagctgtcagtgctttacaaggattgagggagaagacgattggcaagtacgccaagtcagagtttgacaaatgacaactcactcaccatatgtattactacctttgcttcatatgaaaaaaactaacagcgatcatggatcagctatcaaaggtcaaggaattccttaagacttacgcgcagttggatcaagcagtacaagagatggatgacattgagtctcagagagaggaaaagactaattttgatttgtttcaggaagaaggattggagattaaggagaagccttcctattatcgggcagatgaagaagagattgattcagatgaagacagcgtggatgatgcacaagacttagggatacgtacatcaacaagtcccatcgaggggtatgtggatgaggagcaggatgattatgaggatgaggaagtgaacgtggtgtttacatcggactggaaacagcctgagctggaatccgacggggatgggaaaactctccgattgacgataccagatggattgactggggagcagaagtcgcaatggcttgccacgattaaggcagttgttcagagtgctaaatattggaacatctcagaatgttcatttgagagttatgagcaaggggttttgattagagagagacaaatgactcctgatgtctacaaagtcactcctgttttaaatgctccaccggttcaaatgacagctaatcaagatgtttggtctctcagcagcactccatttacatttttgcccaagaaacaaggtgtgactccattgaccatgtccttagaagaactcttcaacacccgaggtgaattcatatctctgggaggaaacgggaaaatgagtcaccgggaggccatcattctagggttgagacacaagaagctctataatcaagccagactaaagtataacttagcttgaatatgaaaaaaactaacagatatcaaaagatatctctaactcagtccattgtgttcagttcaatcatgagctctctcaagaaaattttgggtattaaagggaaagggaagaaatctaagaaattaggtatggctcccccaccctatgaagaagagactccaatggaatattctccaagtgcaccttatgataagtcattgtttggagtcgaagatatggatttccatgatcaacgtcaactccgatatgagaaatttcacttctcattgaagatgactgtgagatcaaacaaaccatttcgaaattatgatgacgttgcagcagcggtgtccaattgggatcatatgtacatcggcatggcaggaaaacgtcctttttataagatattagcattcatgggttctactctattgaaggctacaccagctgtcttggctgaccaaggacagccagaatatcatgctcactgtgagggacgagcttacttgccgcatcggttagggccgacccctccgatgttgaatgtccctgaacattttcgccgtccatttaacatcggattattcagagggacaatcgacataaccctggtacttttcgatgatgaatctgtagattctgccccggtcatatgggatcattttaatgcatccagattgagcagcttcagagaaaaggctttgttgtttggtttgattctagaaaagaaagccactgggaattgggtattggactctattagtcatttcaagtaattatcacaagtgttgaggtgatgggcagactatgaaaaaaactaacagggttcaaacactcttgatcgaggtacccagttatatttgttacaacaatgatttcgaatatgtttttcttgtttcaactctcattatttctacagtttatagcaggagatgagtcattagaaacaataacagcccctgaaactcctgaccctatactcttaaaaggagatacaaaatatctgttcttagtcccttcttctgtcaaaaattggaaaccagctgacctgaatgaattaacatgcccccccctaatctcgaaaccagatacttctgaaatgacttatttttccacagatgtgatggagttacaaaaacatcatgaattggcaccagtagaagggtatttatgttcgggtttgcgttacaaagtaatatgttctgaaggattttttggacaaaaaacaatagcaaaaaagattgagaacattgaacctgatagtaaacaatgccttgatgacttgtcaaaatttaagaatgatgattacctactcccatatttcccttctgaagattgtaattggatgaaagagactcccacccataaagattttatagtttttcaaaaacattttgttaaatatgacccatacaataatggtttttatgatcctttacttaaaaaagactactgtgatactcaagtctgtgagacagaacatgatcaaactatttggataacagaaaagagtattgaaaatgaatgcatcttcaattatccgattaaaaagcatatattccatacagctgactttgggaaaatgataatagattacgaattaaatcaatggacttcagtggaagatgggtgtttaattaactattgtggaagagagggaataaggttatctaatgggatgttctttgtaggtaagttctataaaaatctcaataatttacagacctgtagtgctggaacaaaggtcagttacaagcctttaacctccaagctggaagaaattgaaaatgaaatcattctagatcaggaaagattattatgtcttgattcaattaggcaaatgacagcaacaaaaaaattatcattttattctttatcctttctagaaccaaaatcttctagtaggcacaaggtctttagaattcataataaaacactagaatataccgaaaccgaatggcatccaatcatgtcgtttaattttgatgaaccaaacaaaattggaattgacaagaatggtaaatcagtttattggaatgaatgggttcctagtggaatatctgggctgttatcagggttcaatggagtctacaaaaaagaaaatgaaactaaagtaactattgcccgattagaaacaataaaagaagattatgatagggagatgatgatagatcacgagttggtagaggtagaacatcctaaaattgtacacttaaaaagagagaacatcacaggatctagagtcgaaattgttaataaagaacattctgatgtgagtggttggctgtcatcagtattgagtagtttttggggaaaaatcatgatgacaataataagtataatcttaatcgtaataataggattagttttaataaactgctgcccaattatatgcaaatcatgtattaaacgttataaaacaaaggaagaatcccgcaatagacatagattggatagagaagataacggtagattgaggaggcaacatcgagttatttttaacaatcaatccaatgatgaagaaaatgccattgaaatggtagaatatactgacactcccaggccattgcgaccgattcctgatgccacaacatcagacactgagtcaagatcccccacaacagcccatagttttttcaaccgttaagcggccgccagatgacgcatgagggtcagatcagatttacagcgtaagtgtgatatttaggattataaaggttccttcattttaatttgttacagactgtatgaaaaaaactcatcaacagccatcatggatgttaacgattttgagttgcatgaggactttgcattgtctgaagatgactttgtcacttcagaatttctcaatccggaagaccaaatgacatacctgaatcatgccgattataatttgaattctcccttaatcagcgatgatattgatttcctgatcaagaaatataatcatgagcaaattccgaaaatgtgggatgtcaagaattgggagggagtgttagagatgttgacagcctgtcaagccagtccaattttatctagcactatgcataagtgggtgggaaagtggctcatgtctgatgatcatgacgcaagccaaggcttcagttttcttcatgaagtggacaaagaagctgatctgacgtttgaggtggtggagacattcattagaggatggggaggtcgagaattgcagtacaagaggaaagacacatttccggactcctttagagttgcagcctcattgtgtcaaaaattccttgatttgcacaaactcactctgataatgaattcagtctctgaagtcgaacttaccaacctagcaaagaattttaaaggaaaaaacaggaaagcaaaaagcggaaatctgataaccagattgagggttcccagtttaggtcctgcttttgtgactcagggatgggtgtacatgaagaagttggaaatgattatggatcggaattttttgttgatgttgaaagacgttatcatcgggaggatgcagacgatcctgtccatgatctcaagagatgataatctcttctccgagtctgatatctttactgtattaaagatataccggataggggataagatattagaaaggcaagggacaaagggttacgacttgatcaaaatgattgagcctatttgtaacttaaagatgatgaatctggcacgtaaatatcgtcctctcatccctacatttcctcattttgaaaaacatattgctgactctgttaaggaaggatcgaaaatagacaaagggattgagtttatatatgatcacattatgtcaatccctggtgtggacttgaccttagttatttacggatcatttcggcactggggtcatccttttatcaactactatgagggcttagagaagctacacaagcaggttacaatgcccaagactattgacagagaatatgcagaatgtcttgctagtgatctggcaagaatcgttcttcagcaacaattcaatgaacataagaaatggtttgttgatgtagataaagtcccacaatcccatcctttcaaaagccatatgaaagagaatacttggcctactgcagcccaagttcaggattacggcgatcgctggcatcagctcccactcatcaaatgcttcgaaatcccagatttgttagatccatcgatcatctactcagacaaaagtcattccatgaaccggtctgaagtactacgacatgtaagacttacacctcatgtgcccattccaagcaggaaagtattgcagacaatgttggagactaaggcaacagactggaaagagtttttaaagaaaattgacgaagaggggttagaggatgatgatcttgtcataggactcaaagggaaagagagagaattaaaaattgcgggaagattcttttctttgatgtcctggaagctcagagagtattttgtcatcactgagtatttgattaagacgcactttgtcccgatgtttaaagggttgaccatggcggatgacttgacagcggtgataaagaagatgatggacacatcttcaggacaaggcttagataattatgaatccatttgtatagccaaccatattgactatgagaagtggaacaatcatcaaagaaaagagtcgaacgggcccgtgttcaaggtgatgggtcaattcttgggatatccacgtctgattgagagaactcatgaattttttgagaagagtctgatatattacaatggacgaccagatctgatgcgggttcgaggaaattctctagtcaacgcctcatctttaaatgtctgctgggagggtcaagctgggggattagaaggactgcgacagaagggatggagtattctaaatttgcttgtcattcagagagaagcaaaaataaggaacaccgccgtgaaagtgctagctcaaggtgacaatcaggtgatatgtactcagtataaaacgaagaaatcccggaatgatattgagcttaaggcagctctaacacagatggtatctaataatgagatgattatgtctgcgattaaatcaggcaccgagaaactgggtcttttgattaatgatgatgagacaatgcaatctgctgattacctcaattacgggaaggttcccattttcagaggagtaatcagaggccttgagacaaaaagatggtcacgcgtgacctgtgtgacaaatgatcagattccaacgtgtgcgaacattatgagctctgtgtcaactaatgcattaactgtagcccattttgccgagaatccagtcaatgccatcattcagtataactactttggaacatttgcaaggctactgctgatgatgcatgaccccgctctgaggatctctctgtatgaagtccaatcaaaaattccaggacttcacagtttgacatttaaatattctatgttgtatctggatccttcgataggaggagtctccggaatgtcactctcgagattcctcataagatcatttccagatccagtgacagaaagtttggcgttctggaaatttatccactctcatgcaagaagcgattcattaaaggagatatgtgcagtttttggaaatcctgaaattgcaagatttcggctaactcatgtcgataaattggtggaagacccaacctcattgaacatagctatgggaatgagtcctgctaatctattaaagacagaggtaaaaaaatgtctactggaatcaaggcagagcatcaagaaccagattgtaagagatgctactatttacctacaccatgaggaagacaaacttcgtagtttcttatggtccataacaccactgttccctcggttcttgagtgaattcaaatctgggacattcatcggagtagcagatggcctgatcagcttatttcagaactctaggactattcgaaattcttttaaaaagcgttatcacagggaacttgatgatttaataatcaagagcgaagtttcctcacttatgcatttgggtaagctacatttgaggcgaggctcagttcgtatgtggacttgctcttctactcaggctgatcttctccgattccggtcatggggaagatctgttataggaaccacagtccctcatcccttagagatgttaggacaacattttaaaaaggagactccttgcagtgcttgcaacatatccggattagactatgtatctgtccactgtccgaatgggattcatgacgtttttgaatcacgtggtccactccctgcatatttgggttctaaaacatccgaatcaacttcgatcttgcagccgtgggagagagagagtaaagtaccgttgattaagcgtgccacaaggcttcgtgatgcaatttcatggtttgtgtctcccgactctaacttggcctcaactatccttaagaacataaatgcattaacaggagaagaatggtcaaagaagcagcatggatttaaaaggacgggatcggcgttacacaggttctccacatccaggatgagtcatggtggttttgcttctcagagtacggctgccttgactagattgatggcaactactgacactatgagagatctgggagaacagaactatgatttcctgtttcaggcgacattattgtatgctcaaataaccacaactgtagtcaggaatggatcatttcatagctgcacggaccattaccatataacctgcaaatcttgtctgagggccattgatgagattaccttggattcagcgatggaatatagccctccagatgtatcatcagttttacaatcttggaggaatggagaaggctcttggggacatgaagtgaaacaaatatacccagttgaaggtgactggaggggactatctcctgttgaacaatcttatcaagtcggacgctgtatcgggtttctgttcggtgatctggcgtatagaaaatcatcccatgcagatgatagctccatgtttccgttatctatacaaaacaaagtcagaggaagaggctttttaaaagggcttatggatgggttaatgagagccagttgttgccaggtgatccatcgtcgaagcttagcccatctgaagagaccggctaatgcagtctatggagggctgatttatttgatagacaaattgagtgcatctgccccttttctttcactgacgagacatggacctttaagggaagaattagaaactgttccacataagataccgacttcttatcctacgagcaaccgagatatgggggtgatagttcgtaattattttaaatatcagtgcagactggtagaaaaaggtcggtacaagacacattatcctcaattgtggcttttctcagatgtgctgtccattgatttcttaggacccctgtctatatcttcaactctattgggtattctgtataaacagacgttatcttctcgagacaaaaatgagttgagagaactcgctaacttgtcttcattgttgagatcaggagaaggatgggaagatatccatgtcaaattcttctctaaggacactttactctgccctgaagagatccgacatgcgtgcaaatttgggattgctaaggaatccgctgttttaagctattatcctccttggtctcaagagtcttatggaggcatcacctcgatccccgtatatttttcgaccaggaagtatcccaaaattttagatgtccctcctcgggttcaaaacccattggtctcgggtctacgattggggcaactccctactggagcacattataagattaggagcattgtaaagaacaagaaccttcgttatagagatttccttagttgtggggatggatctggggggatgaccgcggcactattgagagaaaacagacaaagtaggggaatcttcaacagcctgttagagttagccggatctcttatgagaggagcatctccagagcctccaagtgcactggagacgctcgggcaagaacgatctaggtgtgtgaatggaagcacatgttgggagtactcatctgacctaagccaaaaagagacatgggattacttcttaagattgaagagaggcctgggtttgaccgtggacttaatcaccatggacatggaggtcagagaccctaatacaagtttgatgatagaaaagaacctcaaagtttatctgcatcagatattagaaccaactggtgtcttaatatataaaacatacgggacccatattgcgacacaaacagataatatcctgacgataatcggtcctttctttgagacggttgacctagtccagtccgaatacagcagctcacaaacgtccgaggtctattttgtaggacgaggcttgcgctctcatgttgacgaaccctgggtggactggccatccttaatggacaattggagatccatttatgcttttcatgatcctactacagaatttatcagagcaaaaaaagtctgtgaaattgacagtcttataggcattccggctcaattcattccagacccatttgtaaatctcgagaccatgctacagatagttggtgttccaacaggagtttcgcatgccgcagctctattatcatcacaatatccaaatcaattggtcacaacgtcaatattttatatgacactcgtgtcttattataatgtaaaccatattcgaagaagccccaagcctttctctcctccgtctgatggagtctcacagaacattggttcagccatagtcggactaagtttttgggtgagtttgatggagaatgatctcggattatacaaacaggctctaggtgcaataaagacgtcattccctattagatggtcctctgtccagaccaaggatgggtttacacaagaatggagaactaaaggaaacggaattcctaaagattgtcgtctctcagactctttggctcagataggaaactggatcagagcgatggaattggttaggaacaaaacgaggcaatcaggattttctgaaaccctatttgatcaattctgcggacttgcagaccatcacctcaaatggcggaagttgggaaacagaacaggaattattgattggctaaataatagaatttcatccattgacaaatccatcttggtgaccaaaagtgatctgcatgacgagaactcatggagggagtgaagatgtattcttccacctctcattgggtgatacccatatatgaaaaaaactataagtactttaaactctctttgttttttaatgtatatctggttttgttgtttccgt

SEQ ID NO: 10 - RNA, 11380 nt, Artificial
  Chimeric virus containing RNA from Maraba and Bahia Grande viruses
acgaagacaaacaaaccauugauagaauuaagaggcucaugaaaauccuuaacagcguucaaaaugucuguuacagucaagagagucauugaugauucacucaucacccccaaauugccugcgaaugaggacccuguggaguacccugcugauuauuucaaaaagucccgugauauuccgguguacauaaacacgaccaaaaguuugucugauuugcggggcuauguuuaucaaggccuaaagucaggcaacaucucuauaauucaugucaacaguuaucuguaugcagcauuaaaagagaucagaggaaaauuggacagagauuggaucaccuuugguauccaaaucggaaaaacaggagauagcguggggauauucgauuuacugacccuaaaaccucuagaugguguuuuaccagauggggugucugaugcuacucgaacuagcucagacgaugcauggcuuccacuguaucuauugggguuauacagaguuggucgaacacagaugccagaauacaggaagaagcugauggauggucugauuaaucaauguaagaugaucaaugagcaguuugaaccacuguugccagaaggaagagaugucuuugaugucuggggaaaugacagcaauuacacaaagauuguggccgcuguagauauguucuuccauauguucaaaaagcaugagaaggccucuuucagguauggcacaauagugucaagauuuaaggauugugcagcauuggcuacauuuggucaucuguguaagaucacugguauguccacugaagaugugacaacuuggauucuaaacagggagguggcugaugagaugguucaaaugauguacccaggacaggagauagauaaggcugauucuuacaugccuuaucuaaucgacuuaggucuguccucaaaaucuccauauucaucaguuaaaaauccagcuuuccauuuuuggggucaauugaccgcauuguuacugagaucaaccagagccagaaaugcacgucagccggaugacaucgaguauacaucccugaccacugcugggcuguuguaugcauaugccguugguucgucugcagaccuggcucaacaauucuacguuggggacaacaaguaugugccagaaacuggagauggaggauuaaccaccaaugcaccgccacaagggcgagauguggucgaguggcuuaguugguuugaagaucaaaacagaaaaccuaccccagacaugcucauguaugcuaagagagcugucagugcuuuacaaggauugagggagaagacgauuggcaaguacgccaagucagaguuugacaaaugacaacucacucaccauauguauuacuaccuuugcuucauaugaaaaaaacuaacagcgaucauggaucagcuaucaaaggucaaggaauuccuuaagacuuacgcgcaguuggaucaagcaguacaagagauggaugacauugagucucagagagaggaaaagacuaauuuugauuuguuucaggaagaaggauuggagauuaaggagaagccuuccuauuaucgggcagaugaagaagagauugauucagaugaagacagcguggaugaugcacaagacuuagggauacguacaucaacaagucccaucgagggguauguggaugaggagcaggaugauuaugaggaugaggaagugaacgugguguuuacaucggacuggaaacagccugagcuggaauccgacggggaugggaaaacucuccgauugacgauaccagauggauugacuggggagcagaagucgcaauggcuugccacgauuaaggcaguuguucagagugcuaaauauuggaacaucucagaauguucauuugagaguuaugagcaagggguuuugauuagagagagacaaaugacuccugaugucuacaaagucacuccuguuuuaaaugcuccaccgguucaaaugacagcuaaucaagauguuuggucucucagcagcacuccauuuacauuuuugcccaagaaacaaggugugacuccauugaccauguccuuagaagaacucuucaacacccgaggugaauucauaucucugggaggaaacgggaaaaugagucaccgggaggccaucauucuaggguugagacacaagaagcucuauaaucaagccagacuaaaguauaacuuagcuugaauaugaaaaaaacuaacagauaucaaaagauaucucuaacucaguccauuguguucaguucaaucaugagcucucucaagaaaauuuuggguauuaaagggaaagggaagaaaucuaagaaauuagguauggcucccccacccuaugaagaagagacuccaauggaauauucuccaagugcaccuuaugauaagucauuguuuggagucgaagauauggauuuccaugaucaacgucaacuccgauaugagaaauuucacuucucauugaagaugacugugagaucaaacaaaccauuucgaaauuaugaugacguugcagcagcgguguccaauugggaucauauguacaucggcauggcaggaaaacguccuuuuuauaagauauuagcauucauggguucuacucuauugaaggcuacaccagcugucuuggcugaccaaggacagccagaauaucaugcucacugugagggacgagcuuacuugccgcaucgguuagggccgaccccuccgauguugaaugucccugaacauuuucgccguccauuuaacaucggauuauucagagggacaaucgacauaacccugguacuuuucgaugaugaaucuguagauucugccccggucauaugggaucauuuuaaugcauccagauugagcagcuucagagaaaaggcuuuguuguuugguuugauucuagaaaagaaagccacugggaauuggguauuggacucuauuagucauuucaaguaauuaucacaaguguugaggugaugggcagacuaugaaaaaaacuaacaggguucaaacacucuugaucgagguacccaguuauauuuguuacaacaaugauuucgaauauguuuuucuuguuucaacucucauuauuucuacaguuuauagcaggagaugagucauuagaaacaauaacagccccugaaacuccugacccuauacucuuaaaaggagauacaaaauaucuguucuuagucccuucuucugucaaaaauuggaaaccagcugaccugaaugaauuaacaugccccccccuaaucucgaaaccagauacuucugaaaugacuuauuuuuccacagaugugauggaguuacaaaaacaucaugaauuggcaccaguagaaggguauuuauguucggguuugcguuacaaaguaauauguucugaaggauuuuuuggacaaaaaacaauagcaaaaaagauugagaacauugaaccugauaguaaacaaugccuugaugacuugucaaaauuuaagaaugaugauuaccuacucccauauuucccuucugaagauuguaauuggaugaaagagacucccacccauaaagauuuuauaguuuuucaaaaacauuuuguuaaauaugacccauacaauaaugguuuuuaugauccuuuacuuaaaaaagacuacugugauacucaagucugugagacagaacaugaucaaacuauuuggauaacagaaaagaguauugaaaaugaaugcaucuucaauuauccgauuaaaaagcauauauuccauacagcugacuuugggaaaaugauaauagauuacgaauuaaaucaauggacuucaguggaagauggguguuuaauuaacuauuguggaagagagggaauaagguuaucuaaugggauguucuuuguagguaaguucuauaaaaaucucaauaauuuacagaccuguagugcuggaacaaaggucaguuacaagccuuuaaccuccaagcuggaagaaauugaaaaugaaaucauucuagaucaggaaagauuauuaugucuugauucaauuaggcaaaugacagcaacaaaaaaauuaucauuuuauucuuuauccuuucuagaaccaaaaucuucuaguaggcacaaggucuuuagaauucauaauaaaacacuagaauauaccgaaaccgaauggcauccaaucaugucguuuaauuuugaugaaccaaacaaaauuggaauugacaagaaugguaaaucaguuuauuggaaugaauggguuccuaguggaauaucugggcuguuaucaggguucaauggagucuacaaaaaagaaaaugaaacuaaaguaacuauugcccgauuagaaacaauaaaagaagauuaugauagggagaugaugauagaucacgaguugguagagguagaacauccuaaaauuguacacuuaaaaagagagaacaucacaggaucuagagucgaaauuguuaauaaagaacauucugaugugagugguuggcugucaucaguauugaguaguuuuuggggaaaaaucaugaugacaauaauaaguauaaucuuaaucguaauaauaggauuaguuuuaauaaacugcugcccaauuauaugcaaaucauguauuaaacguuauaaaacaaaggaagaaucccgcaauagacauagauuggauagagaagauaacgguagauugaggaggcaacaucgaguuauuuuuaacaaucaauccaaugaugaagaaaaugccauugaaaugguagaauauacugacacucccaggccauugcgaccgauuccugaugccacaacaucagacacugagucaagaucccccacaacagcccauaguuuuuucaaccguuaagcggccgccagaugacgcaugagggucagaucagauuuacagcguaagugugauauuuaggauuauaaagguuccuucauuuuaauuuguuacagacuguaugaaaaaaacucaucaacagccaucauggauguuaacgauuuugaguugcaugaggacuuugcauugucugaagaugacuuugucacuucagaauuucucaauccggaagaccaaaugacauaccugaaucaugccgauuauaauuugaauucucccuuaaucagcgaugauauugauuuccugaucaagaaauauaaucaugagcaaauuccgaaaaugugggaugucaagaauugggagggaguguuagagauguugacagccugucaagccaguccaauuuuaucuagcacuaugcauaagugggugggaaaguggcucaugucugaugaucaugacgcaagccaaggcuucaguuuucuucaugaaguggacaaagaagcugaucugacguuugaggugguggagacauucauuagaggauggggaggucgagaauugcaguacaagaggaaagacacauuuccggacuccuuuagaguugcagccucauugugucaaaaauuccuugauuugcacaaacucacucugauaaugaauucagucucugaagucgaacuuaccaaccuagcaaagaauuuuaaaggaaaaaacaggaaagcaaaaagcggaaaucugauaaccagauugaggguucccaguuuagguccugcuuuugugacucagggauggguguacaugaagaaguuggaaaugauuauggaucggaauuuuuuguugauguugaaagacguuaucaucgggaggaugcagacgauccuguccaugaucucaagagaugauaaucucuucuccgagucugauaucuuuacuguauuaaagauauaccggauaggggauaagauauuagaaaggcaagggacaaaggguuacgacuugaucaaaaugauugagccuauuuguaacuuaaagaugaugaaucuggcacguaaauaucguccucucaucccuacauuuccucauuuugaaaaacauauugcugacucuguuaaggaaggaucgaaaauagacaaagggauugaguuuauauaugaucacauuaugucaaucccugguguggacuugaccuuaguuauuuacggaucauuucggcacuggggucauccuuuuaucaacuacuaugagggcuuagagaagcuacacaagcagguuacaaugcccaagacuauugacagagaauaugcagaaugucuugcuagugaucuggcaagaaucguucuucagcaacaauucaaugaacauaagaaaugguuuguugauguagauaaagucccacaaucccauccuuucaaaagccauaugaaagagaauacuuggccuacugcagcccaaguucaggauuacggcgaucgcuggcaucagcucccacucaucaaaugcuucgaaaucccagauuuguuagauccaucgaucaucuacucagacaaaagucauuccaugaaccggucugaaguacuacgacauguaagacuuacaccucaugugcccauuccaagcaggaaaguauugcagacaauguuggagacuaaggcaacagacuggaaagaguuuuuaaagaaaauugacgaagagggguuagaggaugaugaucuugucauaggacucaaagggaaagagagagaauuaaaaauugcgggaagauucuuuucuuugauguccuggaagcucagagaguauuuugucaucacugaguauuugauuaagacgcacuuugucccgauguuuaaaggguugaccauggcggaugacuugacagcggugauaaagaagaugauggacacaucuucaggacaaggcuuagauaauuaugaauccauuuguauagccaaccauauugacuaugagaaguggaacaaucaucaaagaaaagagucgaacgggcccguguucaaggugaugggucaauucuugggauauccacgucugauugagagaacucaugaauuuuuugagaagagucugauauauuacaauggacgaccagaucugaugcggguucgaggaaauucucuagucaacgccucaucuuuaaaugucugcugggagggucaagcugggggauuagaaggacugcgacagaagggauggaguauucuaaauuugcuugucauucagagagaagcaaaaauaaggaacaccgccgugaaagugcuagcucaaggugacaaucaggugauauguacucaguauaaaacgaagaaaucccggaaugauauugagcuuaaggcagcucuaacacagaugguaucuaauaaugagaugauuaugucugcgauuaaaucaggcaccgagaaacugggucuuuugauuaaugaugaugagacaaugcaaucugcugauuaccucaauuacgggaagguucccauuuucagaggaguaaucagaggccuugagacaaaaagauggucacgcgugaccugugugacaaaugaucagauuccaacgugugcgaacauuaugagcucugugucaacuaaugcauuaacuguagcccauuuugccgagaauccagucaaugccaucauucaguauaacuacuuuggaacauuugcaaggcuacugcugaugaugcaugaccccgcucugaggaucucucuguaugaaguccaaucaaaaauuccaggacuucacaguuugacauuuaaauauucuauguuguaucuggauccuucgauaggaggagucuccggaaugucacucucgagauuccucauaagaucauuuccagauccagugacagaaaguuuggcguucuggaaauuuauccacucucaugcaagaagcgauucauuaaaggagauaugugcaguuuuuggaaauccugaaauugcaagauuucggcuaacucaugucgauaaauugguggaagacccaaccucauugaacauagcuaugggaaugaguccugcuaaucuauuaaagacagagguaaaaaaaugucuacuggaaucaaggcagagcaucaagaaccagauuguaagagaugcuacuauuuaccuacaccaugaggaagacaaacuucguaguuucuuaugguccauaacaccacuguucccucgguucuugagugaauucaaaucugggacauucaucggaguagcagauggccugaucagcuuauuucagaacucuaggacuauucgaaauucuuuuaaaaagcguuaucacagggaacuugaugauuuaauaaucaagagcgaaguuuccucacuuaugcauuuggguaagcuacauuugaggcgaggcucaguucguauguggacuugcucuucuacucaggcugaucuucuccgauuccggucauggggaagaucuguuauaggaaccacagucccucaucccuuagagauguuaggacaacauuuuaaaaaggagacuccuugcagugcuugcaacauauccggauuagacuauguaucuguccacuguccgaaugggauucaugacguuuuugaaucacgugguccacucccugcauauuuggguucuaaaacauccgaaucaacuucgaucuugcagccgugggagagagagaguaaaguaccguugauuaagcgugccacaaggcuucgugaugcaauuucaugguuugugucucccgacucuaacuuggccucaacuauccuuaagaacauaaaugcauuaacaggagaagaauggucaaagaagcagcauggauuuaaaaggacgggaucggcguuacacagguucuccacauccaggaugagucauggugguuuugcuucucagaguacggcugccuugacuagauugauggcaacuacugacacuaugagagaucugggagaacagaacuaugauuuccuguuucaggcgacauuauuguaugcucaaauaaccacaacuguagucaggaauggaucauuucauagcugcacggaccauuaccauauaaccugcaaaucuugucugagggccauugaugagauuaccuuggauucagcgauggaauauagcccuccagauguaucaucaguuuuacaaucuuggaggaauggagaaggcucuuggggacaugaagugaaacaaauauacccaguugaaggugacuggaggggacuaucuccuguugaacaaucuuaucaagucggacgcuguaucggguuucuguucggugaucuggcguauagaaaaucaucccaugcagaugauagcuccauguuuccguuaucuauacaaaacaaagucagaggaagaggcuuuuuaaaagggcuuauggauggguuaaugagagccaguuguugccaggugauccaucgucgaagcuuagcccaucugaagagaccggcuaaugcagucuauggagggcugauuuauuugauagacaaauugagugcaucugccccuuuucuuucacugacgagacauggaccuuuaagggaagaauuagaaacuguuccacauaagauaccgacuucuuauccuacgagcaaccgagauaugggggugauaguucguaauuauuuuaaauaucagugcagacugguagaaaaaggucgguacaagacacauuauccucaauuguggcuuuucucagaugugcuguccauugauuucuuaggaccccugucuauaucuucaacucuauuggguauucuguauaaacagacguuaucuucucgagacaaaaaugaguugagagaacucgcuaacuugucuucauuguugagaucaggagaaggaugggaagauauccaugucaaauucuucucuaaggacacuuuacucugcccugaagagauccgacaugcgugcaaauuugggauugcuaaggaauccgcuguuuuaagcuauuauccuccuuggucucaagagucuuauggaggcaucaccucgauccccguauauuuuucgaccaggaaguaucccaaaauuuuagaugucccuccucggguucaaaacccauuggucucgggucuacgauuggggcaacucccuacuggagcacauuauaagauuaggagcauuguaaagaacaagaaccuucguuauagagauuuccuuaguuguggggauggaucuggggggaugaccgcggcacuauugagagaaaacagacaaaguaggggaaucuucaacagccuguuagaguuagccggaucucuuaugagaggagcaucuccagagccuccaagugcacuggagacgcucgggcaagaacgaucuaggugugugaauggaagcacauguugggaguacucaucugaccuaagccaaaaagagacaugggauuacuucuuaagauugaagagaggccuggguuugaccguggacuuaaucaccauggacauggaggucagagacccuaauacaaguuugaugauagaaaagaaccucaaaguuuaucugcaucagauauuagaaccaacuggugucuuaauauauaaaacauacgggacccauauugcgacacaaacagauaauauccugacgauaaucgguccuuucuuugagacgguugaccuaguccaguccgaauacagcagcucacaaacguccgaggucuauuuuguaggacgaggcuugcgcucucauguugacgaacccuggguggacuggccauccuuaauggacaauuggagauccauuuaugcuuuucaugauccuacuacagaauuuaucagagcaaaaaaagucugugaaauugacagucuuauaggcauuccggcucaauucauuccagacccauuuguaaaucucgagaccaugcuacagauaguugguguuccaacaggaguuucgcaugccgcagcucuauuaucaucacaauauccaaaucaauuggucacaacgucaauauuuuauaugacacucgugucuuauuauaauguaaaccauauucgaagaagccccaagccuuucucuccuccgucugauggagucucacagaacauugguucagccauagucggacuaaguuuuugggugaguuugauggagaaugaucucggauuauacaaacaggcucuaggugcaauaaagacgucauucccuauuagaugguccucuguccagaccaaggauggguuuacacaagaauggagaacuaaaggaaacggaauuccuaaagauugucgucucucagacucuuuggcucagauaggaaacuggaucagagcgauggaauugguuaggaacaaaacgaggcaaucaggauuuucugaaacccuauuugaucaauucugcggacuugcagaccaucaccucaaauggcggaaguugggaaacagaacaggaauuauugauuggcuaaauaauagaauuucauccauugacaaauccaucuuggugaccaaaagugaucugcaugacgagaacucauggagggagugaagauguauucuuccaccucucauugggugauacccauauaugaaaaaaacuauaaguacuuuaaacucucuuuguuuuuuaauguauaucugguuuuguuguuuccgu

SEQ ID NO: 11 - DNA, 11101 bp, Artificial
  DNA encoded by chimeric virus that contains RNA from Maraba and Lymphocytic choriomeningitis viruses
acgaagacaaacaaaccattgatagaattaagaggctcatgaaaatccttaacagcgttcaaaatgtctgttacagtcaagagagtcattgatgattcactcatcacccccaaattgcctgcgaatgaggaccctgtggagtaccctgctgattatttcaaaaagtcccgtgatattccggtgtacataaacacgaccaaaagtttgtctgatttgcggggctatgtttatcaaggcctaaagtcaggcaacatctctataattcatgtcaacagttatctgtatgcagcattaaaagagatcagaggaaaattggacagagattggatcacctttggtatccaaatcggaaaaacaggagatagcgtggggatattcgatttactgaccctaaaacctctagatggtgttttaccagatggggtgtctgatgctactcgaactagctcagacgatgcatggcttccactgtatctattggggttatacagagttggtcgaacacagatgccagaatacaggaagaagctgatggatggtctgattaatcaatgtaagatgatcaatgagcagtttgaaccactgttgccagaaggaagagatgtctttgatgtctggggaaatgacagcaattacacaaagattgtggccgctgtagatatgttcttccatatgttcaaaaagcatgagaaggcctctttcaggtatggcacaatagtgtcaagatttaaggattgtgcagcattggctacatttggtcatctgtgtaagatcactggtatgtccactgaagatgtgacaacttggattctaaacagggaggtggctgatgagatggttcaaatgatgtacccaggacaggagatagataaggctgattcttacatgccttatctaatcgacttaggtctgtcctcaaaatctccatattcatcagttaaaaatccagctttccatttttggggtcaattgaccgcattgttactgagatcaaccagagccagaaatgcacgtcagccggatgacatcgagtatacatccctgaccactgctgggctgttgtatgcatatgccgttggttcgtctgcagacctggctcaacaattctacgttggggacaacaagtatgtgccagaaactggagatggaggattaaccaccaatgcaccgccacaagggcgagatgtggtcgagtggcttagttggtttgaagatcaaaacagaaaacctaccccagacatgctcatgtatgctaagagagctgtcagtgctttacaaggattgagggagaagacgattggcaagtacgccaagtcagagtttgacaaatgacaactcactcaccatatgtattactacctttgcttcatatgaaaaaaactaacagcgatcatggatcagctatcaaaggtcaaggaattccttaagacttacgcgcagttggatcaagcagtacaagagatggatgacattgagtctcagagagaggaaaagactaattttgatttgtttcaggaagaaggattggagattaaggagaagccttcctattatcgggcagatgaagaagagattgattcagatgaagacagcgtggatgatgcacaagacttagggatacgtacatcaacaagtcccatcgaggggtatgtggatgaggagcaggatgattatgaggatgaggaagtgaacgtggtgtttacatcggactggaaacagcctgagctggaatccgacggggatgggaaaactctccgattgacgataccagatggattgactggggagcagaagtcgcaatggcttgccacgattaaggcagttgttcagagtgctaaatattggaacatctcagaatgttcatttgagagttatgagcaaggggttttgattagagagagacaaatgactcctgatgtctacaaagtcactcctgttttaaatgctccaccggttcaaatgacagctaatcaagatgtttggtctctcagcagcactccatttacatttttgcccaagaaacaaggtgtgactccattgaccatgtccttagaagaactcttcaacacccgaggtgaattcatatctctgggaggaaacgggaaaatgagtcaccgggaggccatcattctagggttgagacacaagaagctctataatcaagccagactaaagtataacttagcttgaatatgaaaaaaactaacagatatcaaaagatatctctaactcagtccattgtgttcagttcaatcatgagctctctcaagaaaattttgggtattaaagggaaagggaagaaatctaagaaattaggtatggctcccccaccctatgaagaagagactccaatggaatattctccaagtgcaccttatgataagtcattgtttggagtcgaagatatggatttccatgatcaacgtcaactccgatatgagaaatttcacttctcattgaagatgactgtgagatcaaacaaaccatttcgaaattatgatgacgttgcagcagcggtgtccaattgggatcatatgtacatcggcatggcaggaaaacgtcctttttataagatattagcattcatgggttctactctattgaaggctacaccagctgtcttggctgaccaaggacagccagaatatcatgctcactgtgagggacgagcttacttgccgcatcggttagggccgacccctccgatgttgaatgtccctgaacattttcgccgtccatttaacatcggattattcagagggacaatcgacataaccctggtacttttcgatgatgaatctgtagattctgccccggtcatatgggatcattttaatgcatccagattgagcagcttcagagaaaaggctttgttgtttggtttgattctagaaaagaaagccactgggaattgggtattggactctattagtcatttcaagtaattatcacaagtgttgaggtgatgggcagactatgaaaaaaactaacagggttcaaacactcttgatcgaggtacccagttatatttgttacaacaatgggccagatcgttactatgtttgaagcccttccacacatcattgatgaggtaatcaatatagtgatcattgtgcttattatcattacatctattaaggctgtctacaatttcgcaacctgcggcatcctcgccctggttagattcttgttcctggctggccggtcatgcggtatgtacggactgaacggtcccgacatatacaagggtgtataccaatttaagagcgtagaatttgatatgtctcacctgaacctgacaatgcctaacgcttgcagcgccaacaatagtcaccattatattagtatgggttcatcaggactggagctcactttcacaaacgactccattcttaaccacaacttctgcaacctgacaagcgcctttaacaagaagaccttcgatcacacgctgatgtcaatcgttagttccctccacctgagtatcagagggaattctaaccataaagccgtaagctgcgactttaacaacggcatcacgatccagtacaacttgtctttctccgacccccaatctgccatctcccagtgccgcaccttccgagggcgagtgctcgatatgtttcgcaccgcttttgggggcaagtatatgcgctctggatgggggtgggcaggttcagatgggaaaactacatggtgcagccagaccagttaccagtatcttatcattcaaaatcgcacttgggaaaaccattgtaggtatgccgggcccttcggtatgtcaagaatactgtttgctcaggaaaagactaaattcctgaccagaagactcgccggcacatttacatggacactcagtgattcttctggagtcgagaatccaggaggttattgtctgactaagtggatgattctggctgccgagctgaagtgctttggcaacacagcggttgccaagtgcaatgtcaaccacgatgaagagttttgtgacatgctgagactcatcgattacaataaggctgcgctgtcaaaatttaaacaggacgtcgaaagcgcactccatgttttcaaaactacagtgaattcactgatatcagatcagcttctcatgcgaaaccacctcagagatctcatgggtgttccatactgcaattacagtaaattctggtatctggaacatgccaagacgggagagacgagcgtgcctaagtgctggctggtgactaacgggtcatacctcaacgagacacatttttccgaccagatagagcaggaggcggataacatgataacggagatgctgcgcaaggattacattaaaaggcagggctctaccccccttgcactgatggatttgctcatgttttccacctcagcgtacctgatctctatcttccttcatctcgtgaaaataccgacccacaggcacatcaaagggggttcatgccctaaaccacaccgccttaccaataaggggatttgtagttgtggggcatttaaggtgcctggagtcaaaactatttggaagcggagataagcggccgccagatgacgcatgagggtcagatcagatttacagcgtaagtgtgatatttaggattataaaggttccttcattttaatttgttacagactgtatgaaaaaaactcatcaacagccatcatggatgttaacgattttgagttgcatgaggactttgcattgtctgaagatgactttgtcacttcagaatttctcaatccggaagaccaaatgacatacctgaatcatgccgattataatttgaattctcccttaatcagcgatgatattgatttcctgatcaagaaatataatcatgagcaaattccgaaaatgtgggatgtcaagaattgggagggagtgttagagatgttgacagcctgtcaagccagtccaattttatctagcactatgcataagtgggtgggaaagtggctcatgtctgatgatcatgacgcaagccaaggcttcagttttcttcatgaagtggacaaagaagctgatctgacgtttgaggtggtggagacattcattagaggatggggaggtcgagaattgcagtacaagaggaaagacacatttccggactcctttagagttgcagcctcattgtgtcaaaaattccttgatttgcacaaactcactctgataatgaattcagtctctgaagtcgaacttaccaacctagcaaagaattttaaaggaaaaaacaggaaagcaaaaagcggaaatctgataaccagattgagggttcccagtttaggtcctgcttttgtgactcagggatgggtgtacatgaagaagttggaaatgattatggatcggaattttttgttgatgttgaaagacgttatcatcgggaggatgcagacgatcctgtccatgatctcaagagatgataatctcttctccgagtctgatatctttactgtattaaagatataccggataggggataagatattagaaaggcaagggacaaagggttacgacttgatcaaaatgattgagcctatttgtaacttaaagatgatgaatctggcacgtaaatatcgtcctctcatccctacatttcctcattttgaaaaacatattgctgactctgttaaggaaggatcgaaaatagacaaagggattgagtttatatatgatcacattatgtcaatccctggtgtggacttgaccttagttatttacggatcatttcggcactggggtcatccttttatcaactactatgagggcttagagaagctacacaagcaggttacaatgcccaagactattgacagagaatatgcagaatgtcttgctagtgatctggcaagaatcgttcttcagcaacaattcaatgaacataagaaatggtttgttgatgtagataaagtcccacaatcccatcctttcaaaagccatatgaaagagaatacttggcctactgcagcccaagttcaggattacggcgatcgctggcatcagctcccactcatcaaatgcttcgaaatcccagatttgttagatccatcgatcatctactcagacaaaagtcattccatgaaccggtctgaagtactacgacatgtaagacttacacctcatgtgcccattccaagcaggaaagtattgcagacaatgttggagactaaggcaacagactggaaagagtttttaaagaaaattgacgaagaggggttagaggatgatgatcttgtcataggactcaaagggaaagagagagaattaaaaattgcgggaagattcttttctttgatgtcctggaagctcagagagtattttgtcatcactgagtatttgattaagacgcactttgtcccgatgtttaaagggttgaccatggcggatgacttgacagcggtgataaagaagatgatggacacatcttcaggacaaggcttagataattatgaatccatttgtatagccaaccatattgactatgagaagtggaacaatcatcaaagaaaagagtcgaacgggcccgtgttcaaggtgatgggtcaattcttgggatatccacgtctgattgagagaactcatgaattttttgagaagagtctgatatattacaatggacgaccagatctgatgcgggttcgaggaaattctctagtcaacgcctcatctttaaatgtctgctgggagggtcaagctgggggattagaaggactgcgacagaagggatggagtattctaaatttgcttgtcattcagagagaagcaaaaataaggaacaccgccgtgaaagtgctagctcaaggtgacaatcaggtgatatgtactcagtataaaacgaagaaatcccggaatgatattgagcttaaggcagctctaacacagatggtatctaataatgagatgattatgtctgcgattaaatcaggcaccgagaaactgggtcttttgattaatgatgatgagacaatgcaatctgctgattacctcaattacgggaaggttcccattttcagaggagtaatcagaggccttgagacaaaaagatggtcacgcgtgacctgtgtgacaaatgatcagattccaacgtgtgcgaacattatgagctctgtgtcaactaatgcattaactgtagcccattttgccgagaatccagtcaatgccatcattcagtataactactttggaacatttgcaaggctactgctgatgatgcatgaccccgctctgaggatctctctgtatgaagtccaatcaaaaattccaggacttcacagtttgacatttaaatattctatgttgtatctggatccttcgataggaggagtctccggaatgtcactctcgagattcctcataagatcatttccagatccagtgacagaaagtttggcgttctggaaatttatccactctcatgcaagaagcgattcattaaaggagatatgtgcagtttttggaaatcctgaaattgcaagatttcggctaactcatgtcgataaattggtggaagacccaacctcattgaacatagctatgggaatgagtcctgctaatctattaaagacagaggtaaaaaaatgtctactggaatcaaggcagagcatcaagaaccagattgtaagagatgctactatttacctacaccatgaggaagacaaacttcgtagtttcttatggtccataacaccactgttccctcggttcttgagtgaattcaaatctgggacattcatcggagtagcagatggcctgatcagcttatttcagaactctaggactattcgaaattcttttaaaaagcgttatcacagggaacttgatgatttaataatcaagagcgaagtttcctcacttatgcatttgggtaagctacatttgaggcgaggctcagttcgtatgtggacttgctcttctactcaggctgatcttctccgattccggtcatggggaagatctgttataggaaccacagtccctcatcccttagagatgttaggacaacattttaaaaaggagactccttgcagtgcttgcaacatatccggattagactatgtatctgtccactgtccgaatgggattcatgacgtttttgaatcacgtggtccactccctgcatatttgggttctaaaacatccgaatcaacttcgatcttgcagccgtgggagagagagagtaaagtaccgttgattaagcgtgccacaaggcttcgtgatgcaatttcatggtttgtgtctcccgactctaacttggcctcaactatccttaagaacataaatgcattaacaggagaagaatggtcaaagaagcagcatggatttaaaaggacgggatcggcgttacacaggttctccacatccaggatgagtcatggtggttttgcttctcagagtacggctgccttgactagattgatggcaactactgacactatgagagatctgggagaacagaactatgatttcctgtttcaggcgacattattgtatgctcaaataaccacaactgtagtcaggaatggatcatttcatagctgcacggaccattaccatataacctgcaaatcttgtctgagggccattgatgagattaccttggattcagcgatggaatatagccctccagatgtatcatcagttttacaatcttggaggaatggagaaggctcttggggacatgaagtgaaacaaatatacccagttgaaggtgactggaggggactatctcctgttgaacaatcttatcaagtcggacgctgtatcgggtttctgttcggtgatctggcgtatagaaaatcatcccatgcagatgatagctccatgtttccgttatctatacaaaacaaagtcagaggaagaggctttttaaaagggcttatggatgggttaatgagagccagttgttgccaggtgatccatcgtcgaagcttagcccatctgaagagaccggctaatgcagtctatggagggctgatttatttgatagacaaattgagtgcatctgccccttttctttcactgacgagacatggacctttaagggaagaattagaaactgttccacataagataccgacttcttatcctacgagcaaccgagatatgggggtgatagttcgtaattattttaaatatcagtgcagactggtagaaaaaggtcggtacaagacacattatcctcaattgtggcttttctcagatgtgctgtccattgatttcttaggacccctgtctatatcttcaactctattgggtattctgtataaacagacgttatcttctcgagacaaaaatgagttgagagaactcgctaacttgtcttcattgttgagatcaggagaaggatgggaagatatccatgtcaaattcttctctaaggacactttactctgccctgaagagatccgacatgcgtgcaaatttgggattgctaaggaatccgctgttttaagctattatcctccttggtctcaagagtcttatggaggcatcacctcgatccccgtatatttttcgaccaggaagtatcccaaaattttagatgtccctcctcgggttcaaaacccattggtctcgggtctacgattggggcaactccctactggagcacattataagattaggagcattgtaaagaacaagaaccttcgttatagagatttccttagttgtggggatggatctggggggatgaccgcggcactattgagagaaaacagacaaagtaggggaatcttcaacagcctgttagagttagccggatctcttatgagaggagcatctccagagcctccaagtgcactggagacgctcgggcaagaacgatctaggtgtgtgaatggaagcacatgttgggagtactcatctgacctaagccaaaaagagacatgggattacttcttaagattgaagagaggcctgggtttgaccgtggacttaatcaccatggacatggaggtcagagaccctaatacaagtttgatgatagaaaagaacctcaaagtttatctgcatcagatattagaaccaactggtgtcttaatatataaaacatacgggacccatattgcgacacaaacagataatatcctgacgataatcggtcctttctttgagacggttgacctagtccagtccgaatacagcagctcacaaacgtccgaggtctattttgtaggacgaggcttgcgctctcatgttgacgaaccctgggtggactggccatccttaatggacaattggagatccatttatgcttttcatgatcctactacagaatttatcagagcaaaaaaagtctgtgaaattgacagtcttataggcattccggctcaattcattccagacccatttgtaaatctcgagaccatgctacagatagttggtgttccaacaggagtttcgcatgccgcagctctattatcatcacaatatccaaatcaattggtcacaacgtcaatattttatatgacactcgtgtcttattataatgtaaaccatattcgaagaagccccaagcctttctctcctccgtctgatggagtctcacagaacattggttcagccatagtcggactaagtttttgggtgagtttgatggagaatgatctcggattatacaaacaggctctaggtgcaataaagacgtcattccctattagatggtcctctgtccagaccaaggatgggtttacacaagaatggagaactaaaggaaacggaattcctaaagattgtcgtctctcagactctttggctcagataggaaactggatcagagcgatggaattggttaggaacaaaacgaggcaatcaggattttctgaaaccctatttgatcaattctgcggacttgcagaccatcacctcaaatggcggaagttgggaaacagaacaggaattattgattggctaaataatagaatttcatccattgacaaatccatcttggtgaccaaaagtgatctgcatgacgagaactcatggagggagtgaagatgtattcttccacctctcattgggtgatacccatatatgaaaaaaactataagtactttaaactctctttgttttttaatgtatatctggttttgttgtttccgt

SEQ ID NO: 12 - RNA, 11101 nt, Artificial
  Chimeric virus containing RNA from Maraba and Lymphocytic choriomeningitis viruses
acgaagacaaacaaaccauugauagaauuaagaggcucaugaaaauccuuaacagcguucaaaaugucuguuacagucaagagagucauugaugauucacucaucacccccaaauugccugcgaaugaggacccuguggaguacccugcugauuauuucaaaaagucccgugauauuccgguguacauaaacacgaccaaaaguuugucugauuugcggggcuauguuuaucaaggccuaaagucaggcaacaucucuauaauucaugucaacaguuaucuguaugcagcauuaaaagagaucagaggaaaauuggacagagauuggaucaccuuugguauccaaaucggaaaaacaggagauagcguggggauauucgauuuacugacccuaaaaccucuagaugguguuuuaccagauggggugucugaugcuacucgaacuagcucagacgaugcauggcuuccacuguaucuauugggguuauacagaguuggucgaacacagaugccagaauacaggaagaagcugauggauggucugauuaaucaauguaagaugaucaaugagcaguuugaaccacuguugccagaaggaagagaugucuuugaugucuggggaaaugacagcaauuacacaaagauuguggccgcuguagauauguucuuccauauguucaaaaagcaugagaaggccucuuucagguauggcacaauagugucaagauuuaaggauugugcagcauuggcuacauuuggucaucuguguaagaucacugguauguccacugaagaugugacaacuuggauucuaaacagggagguggcugaugagaugguucaaaugauguacccaggacaggagauagauaaggcugauucuuacaugccuuaucuaaucgacuuaggucuguccucaaaaucuccauauucaucaguuaaaaauccagcuuuccauuuuuggggucaauugaccgcauuguuacugagaucaaccagagccagaaaugcacgucagccggaugacaucgaguauacaucccugaccacugcugggcuguuguaugcauaugccguugguucgucugcagaccuggcucaacaauucuacguuggggacaacaaguaugugccagaaacuggagauggaggauuaaccaccaaugcaccgccacaagggcgagauguggucgaguggcuuaguugguuugaagaucaaaacagaaaaccuaccccagacaugcucauguaugcuaagagagcugucagugcuuuacaaggauugagggagaagacgauuggcaaguacgccaagucagaguuugacaaaugacaacucacucaccauauguauuacuaccuuugcuucauaugaaaaaaacuaacagcgaucauggaucagcuaucaaaggucaaggaauuccuuaagacuuacgcgcaguuggaucaagcaguacaagagauggaugacauugagucucagagagaggaaaagacuaauuuugauuuguuucaggaagaaggauuggagauuaaggagaagccuuccuauuaucgggcagaugaagaagagauugauucagaugaagacagcguggaugaugcacaagacuuagggauacguacaucaacaagucccaucgagggguauguggaugaggagcaggaugauuaugaggaugaggaagugaacgugguguuuacaucggacuggaaacagccugagcuggaauccgacggggaugggaaaacucuccgauugacgauaccagauggauugacuggggagcagaagucgcaauggcuugccacgauuaaggcaguuguucagagugcuaaauauuggaacaucucagaauguucauuugagaguuaugagcaagggguuuugauuagagagagacaaaugacuccugaugucuacaaagucacuccuguuuuaaaugcuccaccgguucaaaugacagcuaaucaagauguuuggucucucagcagcacuccauuuacauuuuugcccaagaaacaaggugugacuccauugaccauguccuuagaagaacucuucaacacccgaggugaauucauaucucugggaggaaacgggaaaaugagucaccgggaggccaucauucuaggguugagacacaagaagcucuauaaucaagccagacuaaaguauaacuuagcuugaauaugaaaaaaacuaacagauaucaaaagauaucucuaacucaguccauuguguucaguucaaucaugagcucucucaagaaaauuuuggguauuaaagggaaagggaagaaaucuaagaaauuagguauggcucccccacccuaugaagaagagacuccaauggaauauucuccaagugcaccuuaugauaagucauuguuuggagucgaagauauggauuuccaugaucaacgucaacuccgauaugagaaauuucacuucucauugaagaugacugugagaucaaacaaaccauuucgaaauuaugaugacguugcagcagcgguguccaauugggaucauauguacaucggcauggcaggaaaacguccuuuuuauaagauauuagcauucauggguucuacucuauugaaggcuacaccagcugucuuggcugaccaaggacagccagaauaucaugcucacugugagggacgagcuuacuugccgcaucgguuagggccgaccccuccgauguugaaugucccugaacauuuucgccguccauuuaacaucggauuauucagagggacaaucgacauaacccugguacuuuucgaugaugaaucuguagauucugccccggucauaugggaucauuuuaaugcauccagauugagcagcuucagagaaaaggcuuuguuguuugguuugauucuagaaaagaaagccacugggaauuggguauuggacucuauuagucauuucaaguaauuaucacaaguguugaggugaugggcagacuaugaaaaaaacuaacaggguucaaacacucuugaucgagguacccaguuauauuuguuacaacaaugggccagaucguuacuauguuugaagcccuuccacacaucauugaugagguaaucaauauagugaucauugugcuuauuaucauuacaucuauuaaggcugucuacaauuucgcaaccugcggcauccucgcccugguuagauucuuguuccuggcuggccggucaugcgguauguacggacugaacggucccgacauauacaaggguguauaccaauuuaagagcguagaauuugauaugucucaccugaaccugacaaugccuaacgcuugcagcgccaacaauagucaccauuauauuaguauggguucaucaggacuggagcucacuuucacaaacgacuccauucuuaaccacaacuucugcaaccugacaagcgccuuuaacaagaagaccuucgaucacacgcugaugucaaucguuaguucccuccaccugaguaucagagggaauucuaaccauaaagccguaagcugcgacuuuaacaacggcaucacgauccaguacaacuugucuuucuccgacccccaaucugccaucucccagugccgcaccuuccgagggcgagugcucgauauguuucgcaccgcuuuugggggcaaguauaugcgcucuggaugggggugggcagguucagaugggaaaacuacauggugcagccagaccaguuaccaguaucuuaucauucaaaaucgcacuugggaaaaccauuguagguaugccgggcccuucgguaugucaagaauacuguuugcucaggaaaagacuaaauuccugaccagaagacucgccggcacauuuacauggacacucagugauucuucuggagucgagaauccaggagguuauugucugacuaaguggaugauucuggcugccgagcugaagugcuuuggcaacacagcgguugccaagugcaaugucaaccacgaugaagaguuuugugacaugcugagacucaucgauuacaauaaggcugcgcugucaaaauuuaaacaggacgucgaaagcgcacuccauguuuucaaaacuacagugaauucacugauaucagaucagcuucucaugcgaaaccaccucagagaucucauggguguuccauacugcaauuacaguaaauucugguaucuggaacaugccaagacgggagagacgagcgugccuaagugcuggcuggugacuaacgggucauaccucaacgagacacauuuuuccgaccagauagagcaggaggcggauaacaugauaacggagaugcugcgcaaggauuacauuaaaaggcagggcucuaccccccuugcacugauggauuugcucauguuuuccaccucagcguaccugaucucuaucuuccuucaucucgugaaaauaccgacccacaggcacaucaaaggggguucaugcccuaaaccacaccgccuuaccaauaaggggauuuguaguuguggggcauuuaaggugccuggagucaaaacuauuuggaagcggagauaagcggccgccagaugacgcaugagggucagaucagauuuacagcguaagugugauauuuaggauuauaaagguuccuucauuuuaauuuguuacagacuguaugaaaaaaacucaucaacagccaucauggauguuaacgauuuugaguugcaugaggacuuugcauugucugaagaugacuuugucacuucagaauuucucaauccggaagaccaaaugacauaccugaaucaugccgauuauaauuugaauucucccuuaaucagcgaugauauugauuuccugaucaagaaauauaaucaugagcaaauuccgaaaaugugggaugucaagaauugggagggaguguuagagauguugacagccugucaagccaguccaauuuuaucuagcacuaugcauaagugggugggaaaguggcucaugucugaugaucaugacgcaagccaaggcuucaguuuucuucaugaaguggacaaagaagcugaucugacguuugaggugguggagacauucauuagaggauggggaggucgagaauugcaguacaagaggaaagacacauuuccggacuccuuuagaguugcagccucauugugucaaaaauuccuugauuugcacaaacucacucugauaaugaauucagucucugaagucgaacuuaccaaccuagcaaagaauuuuaaaggaaaaaacaggaaagcaaaaagcggaaaucugauaaccagauugaggguucccaguuuagguccugcuuuugugacucagggauggguguacaugaagaaguuggaaaugauuauggaucggaauuuuuuguugauguugaaagacguuaucaucgggaggaugcagacgauccuguccaugaucucaagagaugauaaucucuucuccgagucugauaucuuuacuguauuaaagauauaccggauaggggauaagauauuagaaaggcaagggacaaaggguuacgacuugaucaaaaugauugagccuauuuguaacuuaaagaugaugaaucuggcacguaaauaucguccucucaucccuacauuuccucauuuugaaaaacauauugcugacucuguuaaggaaggaucgaaaauagacaaagggauugaguuuauauaugaucacauuaugucaaucccugguguggacuugaccuuaguuauuuacggaucauuucggcacuggggucauccuuuuaucaacuacuaugagggcuuagagaagcuacacaagcagguuacaaugcccaagacuauugacagagaauaugcagaaugucuugcuagugaucuggcaagaaucguucuucagcaacaauucaaugaacauaagaaaugguuuguugauguagauaaagucccacaaucccauccuuucaaaagccauaugaaagagaauacuuggccuacugcagcccaaguucaggauuacggcgaucgcuggcaucagcucccacucaucaaaugcuucgaaaucccagauuuguuagauccaucgaucaucuacucagacaaaagucauuccaugaaccggucugaaguacuacgacauguaagacuuacaccucaugugcccauuccaagcaggaaaguauugcagacaauguuggagacuaaggcaacagacuggaaagaguuuuuaaagaaaauugacgaagagggguuagaggaugaugaucuugucauaggacucaaagggaaagagagagaauuaaaaauugcgggaagauucuuuucuuugauguccuggaagcucagagaguauuuugucaucacugaguauuugauuaagacgcacuuugucccgauguuuaaaggguugaccauggcggaugacuugacagcggugauaaagaagaugauggacacaucuucaggacaaggcuuagauaauuaugaauccauuuguauagccaaccauauugacuaugagaaguggaacaaucaucaaagaaaagagucgaacgggcccguguucaaggugaugggucaauucuugggauauccacgucugauugagagaacucaugaauuuuuugagaagagucugauauauuacaauggacgaccagaucugaugcggguucgaggaaauucucuagucaacgccucaucuuuaaaugucugcugggagggucaagcugggggauuagaaggacugcgacagaagggauggaguauucuaaauuugcuugucauucagagagaagcaaaaauaaggaacaccgccgugaaagugcuagcucaaggugacaaucaggugauauguacucaguauaaaacgaagaaaucccggaaugauauugagcuuaaggcagcucuaacacagaugguaucuaauaaugagaugauuaugucugcgauuaaaucaggcaccgagaaacugggucuuuugauuaaugaugaugagacaaugcaaucugcugauuaccucaauuacgggaagguucccauuuucagaggaguaaucagaggccuugagacaaaaagauggucacgcgugaccugugugacaaaugaucagauuccaacgugugcgaacauuaugagcucugugucaacuaaugcauuaacuguagcccauuuugccgagaauccagucaaugccaucauucaguauaacuacuuuggaacauuugcaaggcuacugcugaugaugcaugaccccgcucugaggaucucucuguaugaaguccaaucaaaaauuccaggacuucacaguuugacauuuaaauauucuauguuguaucuggauccuucgauaggaggagucuccggaaugucacucucgagauuccucauaagaucauuuccagauccagugacagaaaguuuggcguucuggaaauuuauccacucucaugcaagaagcgauucauuaaaggagauaugugcaguuuuuggaaauccugaaauugcaagauuucggcuaacucaugucgauaaauugguggaagacccaaccucauugaacauagcuaugggaaugaguccugcuaaucuauuaaagacagagguaaaaaaaugucuacuggaaucaaggcagagcaucaagaaccagauuguaagagaugcuacuauuuaccuacaccaugaggaagacaaacuucguaguuucuuaugguccauaacaccacuguucccucgguucuugagugaauucaaaucugggacauucaucggaguagcagauggccugaucagcuuauuucagaacucuaggacuauucgaaauucuuuuaaaaagcguuaucacagggaacuugaugauuuaauaaucaagagcgaaguuuccucacuuaugcauuuggguaagcuacauuugaggcgaggcucaguucguauguggacuugcucuucuacucaggcugaucuucuccgauuccggucauggggaagaucuguuauaggaaccacagucccucaucccuuagagauguuaggacaacauuuuaaaaaggagacuccuugcagugcuugcaacauauccggauuagacuauguaucuguccacuguccgaaugggauucaugacguuuuugaaucacgugguccacucccugcauauuuggguucuaaaacauccgaaucaacuucgaucuugcagccgugggagagagagaguaaaguaccguugauuaagcgugccacaaggcuucgugaugcaauuucaugguuugugucucccgacucuaacuuggccucaacuauccuuaagaacauaaaugcauuaacaggagaagaauggucaaagaagcagcauggauuuaaaaggacgggaucggcguuacacagguucuccacauccaggaugagucauggugguuuugcuucucagaguacggcugccuugacuagauugauggcaacuacugacacuaugagagaucugggagaacagaacuaugauuuccuguuucaggcgacauuauuguaugcucaaauaaccacaacuguagucaggaauggaucauuucauagcugcacggaccauuaccauauaaccugcaaaucuugucugagggccauugaugagauuaccuuggauucagcgauggaauauagcccuccagauguaucaucaguuuuacaaucuuggaggaauggagaaggcucuuggggacaugaagugaaacaaauauacccaguugaaggugacuggaggggacuaucuccuguugaacaaucuuaucaagucggacgcuguaucggguuucuguucggugaucuggcguauagaaaaucaucccaugcagaugauagcuccauguuuccguuaucuauacaaaacaaagucagaggaagaggcuuuuuaaaagggcuuauggauggguuaaugagagccaguuguugccaggugauccaucgucgaagcuuagcccaucugaagagaccggcuaaugcagucuauggagggcugauuuauuugauagacaaauugagugcaucugccccuuuucuuucacugacgagacauggaccuuuaagggaagaauuagaaacuguuccacauaagauaccgacuucuuauccuacgagcaaccgagauaugggggugauaguucguaauuauuuuaaauaucagugcagacugguagaaaaaggucgguacaagacacauuauccucaauuguggcuuuucucagaugugcuguccauugauuucuuaggaccccugucuauaucuucaacucuauuggguauucuguauaaacagacguuaucuucucgagacaaaaaugaguugagagaacucgcuaacuugucuucauuguugagaucaggagaaggaugggaagauauccaugucaaauucuucucuaaggacacuuuacucugcccugaagagauccgacaugcgugcaaauuugggauugcuaaggaauccgcuguuuuaagcuauuauccuccuuggucucaagagucuuauggaggcaucaccucgauccccguauauuuuucgaccaggaaguaucccaaaauuuuagaugucccuccucggguucaaaacccauuggucucgggucuacgauuggggcaacucccuacuggagcacauuauaagauuaggagcauuguaaagaacaagaaccuucguuauagagauuuccuuaguuguggggauggaucuggggggaugaccgcggcacuauugagagaaaacagacaaaguaggggaaucuucaacagccuguuagaguuagccggaucucuuaugagaggagcaucuccagagccuccaagugcacuggagacgcucgggcaagaacgaucuaggugugugaauggaagcacauguugggaguacucaucugaccuaagccaaaaagagacaugggauuacuucuuaagauugaagagaggccuggguuugaccguggacuuaaucaccauggacauggaggucagagacccuaauacaaguuugaugauagaaaagaaccucaaaguuuaucugcaucagauauuagaaccaacuggugucuuaauauauaaaacauacgggacccauauugcgacacaaacagauaauauccugacgauaaucgguccuuucuuugagacgguugaccuaguccaguccgaauacagcagcucacaaacguccgaggucuauuuuguaggacgaggcuugcgcucucauguugacgaacccuggguggacuggccauccuuaauggacaauuggagauccauuuaugcuuuucaugauccuacuacagaauuuaucagagcaaaaaaagucugugaaauugacagucuuauaggcauuccggcucaauucauuccagacccauuuguaaaucucgagaccaugcuacagauaguugguguuccaacaggaguuucgcaugccgcagcucuauuaucaucacaauauccaaaucaauuggucacaacgucaauauuuuauaugacacucgugucuuauuauaauguaaaccauauucgaagaagccccaagccuuucucuccuccgucugauggagucucacagaacauugguucagccauagucggacuaaguuuuugggugaguuugauggagaaugaucucggauuauacaaacaggcucuaggugcaauaaagacgucauucccuauuagaugguccucuguccagaccaaggauggguuuacacaagaauggagaacuaaaggaaacggaauuccuaaagauugucgucucucagacucuuuggcucagauaggaaacuggaucagagcgauggaauugguuaggaacaaaacgaggcaaucaggauuuucugaaacccuauuugaucaauucugcggacuugcagaccaucaccucaaauggcggaaguugggaaacagaacaggaauuauugauuggcuaaauaauagaauuucauccauugacaaauccaucuuggugaccaaaagugaucugcaugacgagaacucauggagggagugaagauguauucuuccaccucucauugggugauacccauauaugaaaaaaacuauaaguacuuuaaacucucuuuguuuuuuaauguauaucugguuuuguuguuuccgu

SEQ ID NO: 13 - DNA, 11632 bp, Artificial
  DNA encoded by chimeric virus that contains RNA from Maraba and Ebola viruses
acgaagacaaacaaaccattgatagaattaagaggctcatgaaaatccttaacagcgttcaaaatgtctgttacagtcaagagagtcattgatgattcactcatcacccccaaattgcctgcgaatgaggaccctgtggagtaccctgctgattatttcaaaaagtcccgtgatattccggtgtacataaacacgaccaaaagtttgtctgatttgcggggctatgtttatcaaggcctaaagtcaggcaacatctctataattcatgtcaacagttatctgtatgcagcattaaaagagatcagaggaaaattggacagagattggatcacctttggtatccaaatcggaaaaacaggagatagcgtggggatattcgatttactgaccctaaaacctctagatggtgttttaccagatggggtgtctgatgctactcgaactagctcagacgatgcatggcttccactgtatctattggggttatacagagttggtcgaacacagatgccagaatacaggaagaagctgatggatggtctgattaatcaatgtaagatgatcaatgagcagtttgaaccactgttgccagaaggaagagatgtctttgatgtctggggaaatgacagcaattacacaaagattgtggccgctgtagatatgttcttccatatgttcaaaaagcatgagaaggcctctttcaggtatggcacaatagtgtcaagatttaaggattgtgcagcattggctacatttggtcatctgtgtaagatcactggtatgtccactgaagatgtgacaacttggattctaaacagggaggtggctgatgagatggttcaaatgatgtacccaggacaggagatagataaggctgattcttacatgccttatctaatcgacttaggtctgtcctcaaaatctccatattcatcagttaaaaatccagctttccatttttggggtcaattgaccgcattgttactgagatcaaccagagccagaaatgcacgtcagccggatgacatcgagtatacatccctgaccactgctgggctgttgtatgcatatgccgttggttcgtctgcagacctggctcaacaattctacgttggggacaacaagtatgtgccagaaactggagatggaggattaaccaccaatgcaccgccacaagggcgagatgtggtcgagtggcttagttggtttgaagatcaaaacagaaaacctaccccagacatgctcatgtatgctaagagagctgtcagtgctttacaaggattgagggagaagacgattggcaagtacgccaagtcagagtttgacaaatgacaactcactcaccatatgtattactacctttgcttcatatgaaaaaaactaacagcgatcatggatcagctatcaaaggtcaaggaattccttaagacttacgcgcagttggatcaagcagtacaagagatggatgacattgagtctcagagagaggaaaagactaattttgatttgtttcaggaagaaggattggagattaaggagaagccttcctattatcgggcagatgaagaagagattgattcagatgaagacagcgtggatgatgcacaagacttagggatacgtacatcaacaagtcccatcgaggggtatgtggatgaggagcaggatgattatgaggatgaggaagtgaacgtggtgtttacatcggactggaaacagcctgagctggaatccgacggggatgggaaaactctccgattgacgataccagatggattgactggggagcagaagtcgcaatggcttgccacgattaaggcagttgttcagagtgctaaatattggaacatctcagaatgttcatttgagagttatgagcaaggggttttgattagagagagacaaatgactcctgatgtctacaaagtcactcctgttttaaatgctccaccggttcaaatgacagctaatcaagatgtttggtctctcagcagcactccatttacatttttgcccaagaaacaaggtgtgactccattgaccatgtccttagaagaactcttcaacacccgaggtgaattcatatctctgggaggaaacgggaaaatgagtcaccgggaggccatcattctagggttgagacacaagaagctctataatcaagccagactaaagtataacttagcttgaatatgaaaaaaactaacagatatcaaaagatatctctaactcagtccattgtgttcagttcaatcatgagctctctcaagaaaattttgggtattaaagggaaagggaagaaatctaagaaattaggtatggctcccccaccctatgaagaagagactccaatggaatattctccaagtgcaccttatgataagtcattgtttggagtcgaagatgatttccacgatcaacgtcaactccgatatgagaaatttcacttctcattgaagatgactgtgagatcaaacaaaccatttcgaaattatgatgacgttgcagcagcggtgtccaattgggatcatatgtacatcggcatggcaggaaaacgtcctttttataagatattagcattcatgggttctactctattgaaggctacaccagctgtcttggctgaccaaggacagccagaatatcatgctcactgtgagggacgagcttacttgccgcatcggttagggccgacccctccgatgttgaatgtccctgaacattttcgccgtccatttaacatcggattattcagagggacaatcgacataaccctggtacttttcgatgatgaatctgtagattctgccccggtcatatgggatcattttaatgcatccagattgagcagcttcagagaaaaggctttgttgtttggtttgattctagaaaagaaagccactgggaattgggtattggactctattagtcatttcaagtaattatcacaagtgttgaggtgatgggcagactatgaaaaaaactaacagggttcaaacactcttgatcgaggtacccagttatatttgttacaacaatgggcgttacaggaatattgcagttacctcgtgatcgattcaagaggacatcattctttctttgggtaattatccttttccaaagaacattttccatcccacttggagtcatccacaatagcacattacaggttagtgatgtcgacaaactagtttgtcgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccataaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaggctaccggttttggaaccaatgagacagagtacttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaaacctcactagaaaaattcgcagtgaagagttgtctttcacagttgtatcaaacggagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctaacaacccttgccacaatctccacgagtccccaatccctcacaaccaaaccaggtccggacaacagcacccataatacacccgtgtataaacttgacatctctgaggcaactcaagttgaacaacatcaccgcagaacagacaacgacagcacagcctccgacactccctctgccacgaccgcagccggacccccaaaagcagagaacaccaacacgagcaagagcactgacttcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggagaagaactcgaagagaagcaattgtcaatgctcaacccaaatgcaaccctaatttacattactggactactcaggatgaaggtgctgcaatcggactggcctggataccatatttcgggccagcagccgagggaatttacatagaggggctaatgcacaatcaagatggtttaatctgtgggttgagacagctggccaacgagacgactcaagctcttcaactgttcctgagagccacaactgagctacgcaccttttcaatcctcaaccgtaaggcaattgatttcttgctgcagcgatggggcggcacatgccacattctgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttaggcggccgccagatgacgcatgagggtcagatcagatttacagcgtaagtgtgatatttaggattataaaggttccttcattttaatttgttacagactgtatgaaaaaaactcatcaacagccatcatggatgttaacgattttgagttgcatgaggactttgcattgtctgaagatgactttgtcacttcagaatttctcaatccggaagaccaaatgacatacctgaatcatgccgattataatttgaattctcccttaatcagcgatgatattgatttcctgatcaagaaatataatcatgagcaaattccgaaaatgtgggatgtcaagaattgggagggagtgttagagatgttgacagcctgtcaagccagtccaattttatctagcactatgcataagtgggtgggaaagtggctcatgtctgatgatcatgacgcaagccaaggcttcagttttcttcatgaagtggacaaagaagctgatctgacgtttgaggtggtggagacattcattagaggatggggaggtcgagaattgcagtacaagaggaaagacacatttccggactcctttagagttgcagcctcattgtgtcaaaaattccttgatttgcacaaactcactctgataatgaattcagtctctgaagtcgaacttaccaacctagcaaagaattttaaaggaaaaaacaggaaagcaaaaagcggaaatctgataaccagattgagggttcccagtttaggtcctgcttttgtgactcagggatgggtgtacatgaagaagttggaaatgattatggatcggaattttttgttgatgttgaaagacgttatcatcgggaggatgcagacgatcctgtccatgatctcaagagatgataatctcttctccgagtctgatatctttactgtattaaagatataccggataggggataagatattagaaaggcaagggacaaagggttacgacttgatcaaaatgattgagcctatttgtaacttaaagatgatgaatctggcacgtaaatatcgtcctctcatccctacatttcctcattttgaaaaacatattgctgactctgttaaggaaggatcgaaaatagacaaagggattgagtttatatatgatcacattatgtcaatccctggtgtggacttgaccttagttatttacggatcatttcggcactggggtcatccttttatcaactactatgagggcttagagaagctacacaagcaggttacaatgcccaagactattgacagagaatatgcagaatgtcttgctagtgatctggcaagaatcgttcttcagcaacaattcaatgaacataagaaatggtttgttgatgtagataaagtcccacaatcccatcctttcaaaagccatatgaaagagaatacttggcctactgcagcccaagttcaggattacggcgatcgctggcatcagctcccactcatcaaatgcttcgaaatcccagatttgttagatccatcgatcatctactcagacaaaagtcattccatgaaccggtctgaagtactacgacatgtaagacttacacctcatgtgcccattccaagcaggaaagtattgcagacaatgttggagactaaggcaacagactggaaagagtttttaaagaaaattgacgaagaggggttagaggatgatgatcttgtcataggactcaaagggaaagagagagaattaaaaattgcgggaagattcttttctttgatgtcctggaagctcagagagtattttgtcatcactgagtatttgattaagacgcactttgtcccgatgtttaaagggttgaccatggcggatgacttgacagcggtgataaagaagatgatggacacatcttcaggacaaggcttagataattatgaatccatttgtatagccaaccatattgactatgagaagtggaacaatcatcaaagaaaagagtcgaacgggcccgtgttcaaggtgatgggtcaattcttgggatatccacgtctgattgagagaactcatgaattttttgagaagagtctgatatattacaatggacgaccagatctgatgcgggttcgaggaaattctctagtcaacgcctcatctttaaatgtctgctgggagggtcaagctgggggattagaaggactgcgacagaagggatggagtattctaaatttgcttgtcattcagagagaagcaaaaataaggaacaccgccgtgaaagtgctagctcaaggtgacaatcaggtgatatgtactcagtataaaacgaagaaatcccggaatgatattgagcttaaggcagctctaacacagatggtatctaataatgagatgattatgtctgcgattaaatcaggcaccgagaaactgggtcttttgattaatgatgatgagacaatgcaatctgctgattacctcaattacgggaaggttcccattttcagaggagtaatcagaggccttgagacaaaaagatggtcacgcgtgacctgtgtgacaaatgatcagattccaacgtgtgcgaacattatgagctctgtgtcaactaatgcattaactgtagcccattttgccgagaatccagtcaatgccatcattcagtataactactttggaacatttgcaaggctactgctgatgatgcatgaccccgctctgaggatctctctgtatgaagtccaatcaaaaattccaggacttcacagtttgacatttaaatattctatgttgtatctggatccttcgataggaggagtctccggaatgtcactctcgagattcctcataagatcatttccagatccagtgacagaaagtttggcgttctggaaatttatccactctcatgcaagaagcgattcattaaaggagatatgtgcagtttttggaaatcctgaaattgcaagatttcggctaactcatgtcgataaattggtggaagacccaacctcattgaacatagctatgggaatgagtcctgctaatctattaaagacagaggtaaaaaaatgtctactggaatcaaggcagagcatcaagaaccagattgtaagagatgctactatttacctacaccatgaggaagacaaacttcgtagtttcttatggtccataacaccactgttccctcggttcttgagtgaattcaaatctgggacattcatcggagtagcagatggcctgatcagcttatttcagaactctaggactattcgaaattcttttaaaaagcgttatcacagggaacttgatgatttaataatcaagagcgaagtttcctcacttatgcatttgggtaagctacatttgaggcgaggctcagttcgtatgtggacttgctcttctactcaggctgatcttctccgattccggtcatggggaagatctgttataggaaccacagtccctcatcccttagagatgttaggacaacattttaaaaaggagactccttgcagtgcttgcaacatatccggattagactatgtatctgtccactgtccgaatgggattcatgacgtttttgaatcacgtggtccactccctgcatatttgggttctaaaacatccgaatcaacttcgatcttgcagccgtgggagagagagagtaaagtaccgttgattaagcgtgccacaaggcttcgtgatgcaatttcatggtttgtgtctcccgactctaacttggcctcaactatccttaagaacataaatgcattaacaggagaagaatggtcaaagaagcagcatggatttaaaaggacgggatcggcgttacacaggttctccacatccaggatgagtcatggtggttttgcttctcagagtacggctgccttgactagattgatggcaactactgacactatgagagatctgggagaacagaactatgatttcctgtttcaggcgacattattgtatgctcaaataaccacaactgtagtcaggaatggatcatttcatagctgcacggaccattaccatataacctgcaaatcttgtctgagggccattgatgagattaccttggattcagcgatggaatatagccctccagatgtatcatcagttttacaatcttggaggaatggagaaggctcttggggacatgaagtgaaacaaatatacccagttgaaggtgactggaggggactatctcctgttgaacaatcttatcaagtcggacgctgtatcgggtttctgttcggtgatctggcgtatagaaaatcatcccatgcagatgatagctccatgtttccgttatctatacaaaacaaagtcagaggaagaggctttttaaaagggcttatggatgggttaatgagagccagttgttgccaggtgatccatcgtcgaagcttagcccatctgaagagaccggctaatgcagtctatggagggctgatttatttgatagacaaattgagtgcatctgccccttttctttcactgacgagacatggacctttaagggaagaattagaaactgttccacataagataccgacttcttatcctacgagcaaccgagatatgggggtgatagttcgtaattattttaaatatcagtgcagactggtagaaaaaggtcggtacaagacacattatcctcaattgtggcttttctcagatgtgctgtccattgatttcttaggacccctgtctatatcttcaactctattgggtattctgtataaacagacgttatcttctcgagacaaaaatgagttgagagaactcgctaacttgtcttcattgttgagatcaggagaaggatgggaagatatccatgtcaaattcttctctaaggacactttactctgccctgaagagatccgacatgcgtgcaaatttgggattgctaaggaatccgctgttttaagctattatcctccttggtctcaagagtcttatggaggcatcacctcgatccccgtatatttttcgaccaggaagtatcccaaaattttagatgtccctcctcgggttcaaaacccattggtctcgggtctacgattggggcaactccctactggagcacattataagattaggagcattgtaaagaacaagaaccttcgttatagagatttccttagttgtggggatggatctggggggatgaccgcggcactattgagagaaaacagacaaagtaggggaatcttcaacagcctgttagagttagccggatctcttatgagaggagcatctccagagcctccaagtgcactggagacgctcgggcaagaacgatctaggtgtgtgaatggaagcacatgttgggagtactcatctgacctaagccaaaaagagacatgggattacttcttaagattgaagagaggcctgggtttgaccgtggacttaatcaccatggacatggaggtcagagaccctaatacaagtttgatgatagaaaagaacctcaaagtttatctgcatcagatattagaaccaactggtgtcttaatatataaaacatacgggacccatattgcgacacaaacagataatatcctgacgataatcggtcctttctttgagacggttgacctagtccagtccgaatacagcagctcacaaacgtccgaggtctattttgtaggacgaggcttgcgctctcatgttgacgaaccctgggtggactggccatccttaatggacaattggagatccatttatgcttttcatgatcctactacagaatttatcagagcaaaaaaagtctgtgaaattgacagtcttataggcattccggctcaattcattccagacccatttgtaaatctcgagaccatgctacagatagttggtgttccaacaggagtttcgcatgccgcagctctattatcatcacaatatccaaatcaattggtcacaacgtcaatattttatatgacactcgtgtcttattataatgtaaaccatattcgaagaagccccaagcctttctctcctccgtctgatggagtctcacagaacattggttcagccatagtcggactaagtttttgggtgagtttgatggagaatgatctcggattatacaaacaggctctaggtgcaataaagacgtcattccctattagatggtcctctgtccagaccaaggatgggtttacacaagaatggagaactaaaggaaacggaattcctaaagattgtcgtctctcagactctttggctcagataggaaactggatcagagcgatggaattggttaggaacaaaacgaggcaatcaggattttctgaaaccctatttgatcaattctgcggacttgcagaccatcacctcaaatggcggaagttgggaaacagaacaggaattattgattggctaaataatagaatttcatccattgacaaatccatcttggtgaccaaaagtgatctgcatgacgagaactcatggagggagtgaagatgtattcttccacctctcattgggtgatacccatatatgaaaaaaactataagtactttaaactctctttgttttttaatgtatatctggttttgttgtttccgt

SEQ ID NO: 14 - RNA, 11632 nt, Artificial
  Chimeric virus containing RNA from Maraba and Ebola viruses
acgaagacaaacaaaccauugauagaauuaagaggcucaugaaaauccuuaacagcguucaaaaugucuguuacagucaagagagucauugaugauucacucaucacccccaaauugccugcgaaugaggacccuguggaguacccugcugauuauuucaaaaagucccgugauauuccgguguacauaaacacgaccaaaaguuugucugauuugcggggcuauguuuaucaaggccuaaagucaggcaacaucucuauaauucaugucaacaguuaucuguaugcagcauuaaaagagaucagaggaaaauuggacagagauuggaucaccuuugguauccaaaucggaaaaacaggagauagcguggggauauucgauuuacugacccuaaaaccucuagaugguguuuuaccagauggggugucugaugcuacucgaacuagcucagacgaugcauggcuuccacuguaucuauugggguuauacagaguuggucgaacacagaugccagaauacaggaagaagcugauggauggucugauuaaucaauguaagaugaucaaugagcaguuugaaccacuguugccagaaggaagagaugucuuugaugucuggggaaaugacagcaauuacacaaagauuguggccgcuguagauauguucuuccauauguucaaaaagcaugagaaggccucuuucagguauggcacaauagugucaagauuuaaggauugugcagcauuggcuacauuuggucaucuguguaagaucacugguauguccacugaagaugugacaacuuggauucuaaacagggagguggcugaugagaugguucaaaugauguacccaggacaggagauagauaaggcugauucuuacaugccuuaucuaaucgacuuaggucuguccucaaaaucuccauauucaucaguuaaaaauccagcuuuccauuuuuggggucaauugaccgcauuguuacugagaucaaccagagccagaaaugcacgucagccggaugacaucgaguauacaucccugaccacugcugggcuguuguaugcauaugccguugguucgucugcagaccuggcucaacaauucuacguuggggacaacaaguaugugccagaaacuggagauggaggauuaaccaccaaugcaccgccacaagggcgagauguggucgaguggcuuaguugguuugaagaucaaaacagaaaaccuaccccagacaugcucauguaugcuaagagagcugucagugcuuuacaaggauugagggagaagacgauuggcaaguacgccaagucagaguuugacaaaugacaacucacucaccauauguauuacuaccuuugcuucauaugaaaaaaacuaacagcgaucauggaucagcuaucaaaggucaaggaauuccuuaagacuuacgcgcaguuggaucaagcaguacaagagauggaugacauugagucucagagagaggaaaagacuaauuuugauuuguuucaggaagaaggauuggagauuaaggagaagccuuccuauuaucgggcagaugaagaagagauugauucagaugaagacagcguggaugaugcacaagacuuagggauacguacaucaacaagucccaucgagggguauguggaugaggagcaggaugauuaugaggaugaggaagugaacgugguguuuacaucggacuggaaacagccugagcuggaauccgacggggaugggaaaacucuccgauugacgauaccagauggauugacuggggagcagaagucgcaauggcuugccacgauuaaggcaguuguucagagugcuaaauauuggaacaucucagaauguucauuugagaguuaugagcaagggguuuugauuagagagagacaaaugacuccugaugucuacaaagucacuccuguuuuaaaugcuccaccgguucaaaugacagcuaaucaagauguuuggucucucagcagcacuccauuuacauuuuugcccaagaaacaaggugugacuccauugaccauguccuuagaagaacucuucaacacccgaggugaauucauaucucugggaggaaacgggaaaaugagucaccgggaggccaucauucuaggguugagacacaagaagcucuauaaucaagccagacuaaaguauaacuuagcuugaauaugaaaaaaacuaacagauaucaaaagauaucucuaacucaguccauuguguucaguucaaucaugagcucucucaagaaaauuuuggguauuaaagggaaagggaagaaaucuaagaaauuagguauggcucccccacccuaugaagaagagacuccaauggaauauucuccaagugcaccuuaugauaagucauuguuuggagucgaagaugauuuccacgaucaacgucaacuccgauaugagaaauuucacuucucauugaagaugacugugagaucaaacaaaccauuucgaaauuaugaugacguugcagcagcgguguccaauugggaucauauguacaucggcauggcaggaaaacguccuuuuuauaagauauuagcauucauggguucuacucuauugaaggcuacaccagcugucuuggcugaccaaggacagccagaauaucaugcucacugugagggacgagcuuacuugccgcaucgguuagggccgaccccuccgauguugaaugucccugaacauuuucgccguccauuuaacaucggauuauucagagggacaaucgacauaacccugguacuuuucgaugaugaaucuguagauucugccccggucauaugggaucauuuuaaugcauccagauugagcagcuucagagaaaaggcuuuguuguuugguuugauucuagaaaagaaagccacugggaauuggguauuggacucuauuagucauuucaaguaauuaucacaaguguugaggugaugggcagacuaugaaaaaaacuaacaggguucaaacacucuugaucgagguacccaguuauauuuguuacaacaaugggcguuacaggaauauugcaguuaccucgugaucgauucaagaggacaucauucuuucuuuggguaauuauccuuuuccaaagaacauuuuccaucccacuuggagucauccacaauagcacauuacagguuagugaugucgacaaacuaguuugucgugacaaacugucauccacaaaucaauugagaucaguuggacugaaucucgaagggaauggaguggcaacugacgugccaucugcaacuaaaagauggggcuucagguccggugucccaccaaagguggucaauuaugaagcuggugaaugggcugaaaacugcuacaaucuugaaaucaaaaaaccugacgggagugagugucuaccagcagcgccagacgggauucggggcuucccccggugccgguaugugcacaaaguaucaggaacgggaccgugugccggagacuuugccuuccauaaagagggugcuuucuuccuguaugaucgacuugcuuccacaguuaucuaccgaggaacgacuuucgcugaaggugucguugcauuucugauacugccccaagcuaagaaggacuucuucagcucacaccccuugagagagccggucaaugcaacggaggacccgucuaguggcuacuauucuaccacaauuagauaucaggcuaccgguuuuggaaccaaugagacagaguacuuguucgagguugacaauuugaccuacguccaacuugaaucaagauucacaccacaguuucugcuccagcugaaugagacaauauauacaagugggaaaaggagcaauaccacgggaaaacuaauuuggaaggucaaccccgaaauugauacaacaaucggggagugggccuucugggaaacuaaaaaaaaccucacuagaaaaauucgcagugaagaguugucuuucacaguuguaucaaacggagccaaaaacaucaguggucagaguccggcgcgaacuucuuccgacccagggaccaacacaacaacugaagaccacaaaaucauggcuucagaaaauuccucugcaaugguucaagugcacagucaaggaagggaagcugcagugucgcaucuaacaacccuugccacaaucuccacgaguccccaaucccucacaaccaaaccagguccggacaacagcacccauaauacacccguguauaaacuugacaucucugaggcaacucaaguugaacaacaucaccgcagaacagacaacgacagcacagccuccgacacucccucugccacgaccgcagccggacccccaaaagcagagaacaccaacacgagcaagagcacugacuuccuggaccccgccaccacaacaaguccccaaaaccacagcgagaccgcuggcaacaacaacacucaucaccaagauaccggagaagagagugccagcagcgggaagcuaggcuuaauuaccaauacuauugcuggagucgcaggacugaucacaggcgggagaagaacucgaagagaagcaauugucaaugcucaacccaaaugcaacccuaauuuacauuacuggacuacucaggaugaaggugcugcaaucggacuggccuggauaccauauuucgggccagcagccgagggaauuuacauagaggggcuaaugcacaaucaagaugguuuaaucuguggguugagacagcuggccaacgagacgacucaagcucuucaacuguuccugagagccacaacugagcuacgcaccuuuucaauccucaaccguaaggcaauugauuucuugcugcagcgauggggcggcacaugccacauucugggaccggacugcuguaucgaaccacaugauuggaccaagaacauaacagacaaaauugaucagauuauucaugauuuuguugauaaaacccuuccggaccagggggacaaugacaauugguggacaggauggagacaauggauaccggcagguauuggaguuacaggcguuauaauugcaguuaucgcuuuauucuguauaugcaaauuugucuuuuaggcggccgccagaugacgcaugagggucagaucagauuuacagcguaagugugauauuuaggauuauaaagguuccuucauuuuaauuuguuacagacuguaugaaaaaaacucaucaacagccaucauggauguuaacgauuuugaguugcaugaggacuuugcauugucugaagaugacuuugucacuucagaauuucucaauccggaagaccaaaugacauaccugaaucaugccgauuauaauuugaauucucccuuaaucagcgaugauauugauuuccugaucaagaaauauaaucaugagcaaauuccgaaaaugugggaugucaagaauugggagggaguguuagagauguugacagccugucaagccaguccaauuuuaucuagcacuaugcauaagugggugggaaaguggcucaugucugaugaucaugacgcaagccaaggcuucaguuuucuucaugaaguggacaaagaagcugaucugacguuugaggugguggagacauucauuagaggauggggaggucgagaauugcaguacaagaggaaagacacauuuccggacuccuuuagaguugcagccucauugugucaaaaauuccuugauuugcacaaacucacucugauaaugaauucagucucugaagucgaacuuaccaaccuagcaaagaauuuuaaaggaaaaaacaggaaagcaaaaagcggaaaucugauaaccagauugaggguucccaguuuagguccugcuuuugugacucagggauggguguacaugaagaaguuggaaaugauuauggaucggaauuuuuuguugauguugaaagacguuaucaucgggaggaugcagacgauccuguccaugaucucaagagaugauaaucucuucuccgagucugauaucuuuacuguauuaaagauauaccggauaggggauaagauauuagaaaggcaagggacaaaggguuacgacuugaucaaaaugauugagccuauuuguaacuuaaagaugaugaaucuggcacguaaauaucguccucucaucccuacauuuccucauuuugaaaaacauauugcugacucuguuaaggaaggaucgaaaauagacaaagggauugaguuuauauaugaucacauuaugucaaucccugguguggacuugaccuuaguuauuuacggaucauuucggcacuggggucauccuuuuaucaacuacuaugagggcuuagagaagcuacacaagcagguuacaaugcccaagacuauugacagagaauaugcagaaugucuugcuagugaucuggcaagaaucguucuucagcaacaauucaaugaacauaagaaaugguuuguugauguagauaaagucccacaaucccauccuuucaaaagccauaugaaagagaauacuuggccuacugcagcccaaguucaggauuacggcgaucgcuggcaucagcucccacucaucaaaugcuucgaaaucccagauuuguuagauccaucgaucaucuacucagacaaaagucauuccaugaaccggucugaaguacuacgacauguaagacuuacaccucaugugcccauuccaagcaggaaaguauugcagacaauguuggagacuaaggcaacagacuggaaagaguuuuuaaagaaaauugacgaagagggguuagaggaugaugaucuugucauaggacucaaagggaaagagagagaauuaaaaauugcgggaagauucuuuucuuugauguccuggaagcucagagaguauuuugucaucacugaguauuugauuaagacgcacuuugucccgauguuuaaaggguugaccauggcggaugacuugacagcggugauaaagaagaugauggacacaucuucaggacaaggcuuagauaauuaugaauccauuuguauagccaaccauauugacuaugagaaguggaacaaucaucaaagaaaagagucgaacgggcccguguucaaggugaugggucaauucuugggauauccacgucugauugagagaacucaugaauuuuuugagaagagucugauauauuacaauggacgaccagaucugaugcggguucgaggaaauucucuagucaacgccucaucuuuaaaugucugcugggagggucaagcugggggauuagaaggacugcgacagaagggauggaguauucuaaauuugcuugucauucagagagaagcaaaaauaaggaacaccgccgugaaagugcuagcucaaggugacaaucaggugauauguacucaguauaaaacgaagaaaucccggaaugauauugagcuuaaggcagcucuaacacagaugguaucuaauaaugagaugauuaugucugcgauuaaaucaggcaccgagaaacugggucuuuugauuaaugaugaugagacaaugcaaucugcugauuaccucaauuacgggaagguucccauuuucagaggaguaaucagaggccuugagacaaaaagauggucacgcgugaccugugugacaaaugaucagauuccaacgugugcgaacauuaugagcucugugucaacuaaugcauuaacuguagcccauuuugccgagaauccagucaaugccaucauucaguauaacuacuuuggaacauuugcaaggcuacugcugaugaugcaugaccccgcucugaggaucucucuguaugaaguccaaucaaaaauuccaggacuucacaguuugacauuuaaauauucuauguuguaucuggauccuucgauaggaggagucuccggaaugucacucucgagauuccucauaagaucauuuccagauccagugacagaaaguuuggcguucuggaaauuuauccacucucaugcaagaagcgauucauuaaaggagauaugugcaguuuuuggaaauccugaaauugcaagauuucggcuaacucaugucgauaaauugguggaagacccaaccucauugaacauagcuaugggaaugaguccugcuaaucuauuaaagacagagguaaaaaaaugucuacuggaaucaaggcagagcaucaagaaccagauuguaagagaugcuacuauuuaccuacaccaugaggaagacaaacuucguaguuucuuaugguccauaacaccacuguucccucgguucuugagugaauucaaaucugggacauucaucggaguagcagauggccugaucagcuuauuucagaacucuaggacuauucgaaauucuuuuaaaaagcguuaucacagggaacuugaugauuuaauaaucaagagcgaaguuuccucacuuaugcauuuggguaagcuacauuugaggcgaggcucaguucguauguggacuugcucuucuacucaggcugaucuucuccgauuccggucauggggaagaucuguuauaggaaccacagucccucaucccuuagagauguuaggacaacauuuuaaaaaggagacuccuugcagugcuugcaacauauccggauuagacuauguaucuguccacuguccgaaugggauucaugacguuuuugaaucacgugguccacucccugcauauuuggguucuaaaacauccgaaucaacuucgaucuugcagccgugggagagagagaguaaaguaccguugauuaagcgugccacaaggcuucgugaugcaauuucaugguuugugucucccgacucuaacuuggccucaacuauccuuaagaacauaaaugcauuaacaggagaagaauggucaaagaagcagcauggauuuaaaaggacgggaucggcguuacacagguucuccacauccaggaugagucauggugguuuugcuucucagaguacggcugccuugacuagauugauggcaacuacugacacuaugagagaucugggagaacagaacuaugauuuccuguuucaggcgacauuauuguaugcucaaauaaccacaacuguagucaggaauggaucauuucauagcugcacggaccauuaccauauaaccugcaaaucuugucugagggccauugaugagauuaccuuggauucagcgauggaauauagcccuccagauguaucaucaguuuuacaaucuuggaggaauggagaaggcucuuggggacaugaagugaaacaaauauacccaguugaaggugacuggaggggacuaucuccuguugaacaaucuuaucaagucggacgcuguaucggguuucuguucggugaucuggcguauagaaaaucaucccaugcagaugauagcuccauguuuccguuaucuauacaaaacaaagucagaggaagaggcuuuuuaaaagggcuuauggauggguuaaugagagccaguuguugccaggugauccaucgucgaagcuuagcccaucugaagagaccggcuaaugcagucuauggagggcugauuuauuugauagacaaauugagugcaucugccccuuuucuuucacugacgagacauggaccuuuaagggaagaauuagaaacuguuccacauaagauaccgacuucuuauccuacgagcaaccgagauaugggggugauaguucguaauuauuuuaaauaucagugcagacugguagaaaaaggucgguacaagacacauuauccucaauuguggcuuuucucagaugugcuguccauugauuucuuaggaccccugucuauaucuucaacucuauuggguauucuguauaaacagacguuaucuucucgagacaaaaaugaguugagagaacucgcuaacuugucuucauuguugagaucaggagaaggaugggaagauauccaugucaaauucuucucuaaggacacuuuacucugcccugaagagauccgacaugcgugcaaauuugggauugcuaaggaauccgcuguuuuaagcuauuauccuccuuggucucaagagucuuauggaggcaucaccucgauccccguauauuuuucgaccaggaaguaucccaaaauuuuagaugucccuccucggguucaaaacccauuggucucgggucuacgauuggggcaacucccuacuggagcacauuauaagauuaggagcauuguaaagaacaagaaccuucguuauagagauuuccuuaguuguggggauggaucuggggggaugaccgcggcacuauugagagaaaacagacaaaguaggggaaucuucaacagccuguuagaguuagccggaucucuuaugagaggagcaucuccagagccuccaagugcacuggagacgcucgggcaagaacgaucuaggugugugaauggaagcacauguugggaguacucaucugaccuaagccaaaaagagacaugggauuacuucuuaagauugaagagaggccuggguuugaccguggacuuaaucaccauggacauggaggucagagacccuaauacaaguuugaugauagaaaagaaccucaaaguuuaucugcaucagauauuagaaccaacuggugucuuaauauauaaaacauacgggacccauauugcgacacaaacagauaauauccugacgauaaucgguccuuucuuugagacgguugaccuaguccaguccgaauacagcagcucacaaacguccgaggucuauuuuguaggacgaggcuugcgcucucauguugacgaacccuggguggacuggccauccuuaauggacaauuggagauccauuuaugcuuuucaugauccuacuacagaauuuaucagagcaaaaaaagucugugaaauugacagucuuauaggcauuccggcucaauucauuccagacccauuuguaaaucucgagaccaugcuacagauaguugguguuccaacaggaguuucgcaugccgcagcucuauuaucaucacaauauccaaaucaauuggucacaacgucaauauuuuauaugacacucgugucuuauuauaauguaaaccauauucgaagaagccccaagccuuucucuccuccgucugauggagucucacagaacauugguucagccauagucggacuaaguuuuugggugaguuugauggagaaugaucucggauuauacaaacaggcucuaggugcaauaaagacgucauucccuauuagaugguccucuguccagaccaaggauggguuuacacaagaauggagaacuaaaggaaacggaauuccuaaagauugucgucucucagacucuuuggcucagauaggaaacuggaucagagcgauggaauugguuaggaacaaaacgaggcaaucaggauuuucugaaacccuauuugaucaauucugcggacuugcagaccaucaccucaaauggcggaaguugggaaacagaacaggaauuauugauuggcuaaauaauagaauuucauccauugacaaauccaucuuggugaccaaaagugaucugcaugacgagaacucauggagggagugaagauguauucuuccaccucucauugggugauacccauauaugaaaaaaacuauaaguacuuuaaacucucuuuguuuuuuaauguauaucugguuuuguuguuuccgu

SEQ ID NO: 15 - DNA, 11719 bp, Artificial
  DNA encoded by chimeric virus that contains RNA from Maraba and Farmington viruses
acgaagacaaacaaaccattgatagaattaagaggctcatgaaaatccttaacagcgttcaaaatgtctgttacagtcaagagagtcattgatgattcactcatcacccccaaattgcctgcgaatgaggaccctgtggagtaccctgctgattatttcaaaaagtcccgtgatattccggtgtacataaacacgaccaaaagtttgtctgatttgcggggctatgtttatcaaggcctaaagtcaggcaacatctctataattcatgtcaacagttatctgtatgcagcattaaaagagatcagaggaaaattggacagagattggatcacctttggtatccaaatcggaaaaacaggagatagcgtggggatattcgatttactgaccctaaaacctctagatggtgttttaccagatggggtgtctgatgctactcgaactagctcagacgatgcatggcttccactgtatctattggggttatacagagttggtcgaacacagatgccagaatacaggaagaagctgatggatggtctgattaatcaatgtaagatgatcaatgagcagtttgaaccactgttgccagaaggaagagatgtctttgatgtctggggaaatgacagcaattacacaaagattgtggccgctgtagatatgttcttccatatgttcaaaaagcatgagaaggcctctttcaggtatggcacaatagtgtcaagatttaaggattgtgcagcattggctacatttggtcatctgtgtaagatcactggtatgtccactgaagatgtgacaacttggattctaaacagggaggtggctgatgagatggttcaaatgatgtacccaggacaggagatagataaggctgattcttacatgccttatctaatcgacttaggtctgtcctcaaaatctccatattcatcagttaaaaatccagctttccatttttggggtcaattgaccgcattgttactgagatcaaccagagccagaaatgcacgtcagccggatgacatcgagtatacatccctgaccactgctgggctgttgtatgcatatgccgttggttcgtctgcagacctggctcaacaattctacgttggggacaacaagtatgtgccagaaactggagatggaggattaaccaccaatgcaccgccacaagggcgagatgtggtcgagtggcttagttggtttgaagatcaaaacagaaaacctaccccagacatgctcatgtatgctaagagagctgtcagtgctttacaaggattgagggagaagacgattggcaagtacgccaagtcagagtttgacaaatgacaactcactcaccatatgtattactacctttgcttcatatgaaaaaaactaacagcgatcatggatcagctatcaaaggtcaaggaattccttaagacttacgcgcagttggatcaagcagtacaagagatggatgacattgagtctcagagagaggaaaagactaattttgatttgtttcaggaagaaggattggagattaaggagaagccttcctattatcgggcagatgaagaagagattgattcagatgaagacagcgtggatgatgcacaagacttagggatacgtacatcaacaagtcccatcgaggggtatgtggatgaggagcaggatgattatgaggatgaggaagtgaacgtggtgtttacatcggactggaaacagcctgagctggaatccgacggggatgggaaaactctccgattgacgataccagatggattgactggggagcagaagtcgcaatggcttgccacgattaaggcagttgttcagagtgctaaatattggaacatctcagaatgttcatttgagagttatgagcaaggggttttgattagagagagacaaatgactcctgatgtctacaaagtcactcctgttttaaatgctccaccggttcaaatgacagctaatcaagatgtttggtctctcagcagcactccatttacatttttgcccaagaaacaaggtgtgactccattgaccatgtccttagaagaactcttcaacacccgaggtgaattcatatctctgggaggaaacgggaaaatgagtcaccgggaggccatcattctagggttgagacacaagaagctctataatcaagccagactaaagtataacttagcttgaatatgaaaaaaactaacagatatcaaaagatatctctaactcagtccattgtgttcagttcaatcatgagctctctcaagaaaattttgggtattaaagggaaagggaagaaatctaagaaattaggtatggctcccccaccctatgaagaagagactccaatggaatattctccaagtgcaccttatgataagtcattgtttggagtcgaagatatggatttccatgatcaacgtcaactccgatatgagaaatttcacttctcattgaagatgactgtgagatcaaacaaaccatttcgaaattatgatgacgttgcagcagcggtgtccaattgggatcatatgtacatcggcatggcaggaaaacgtcctttttataagatattagcattcatgggttctactctattgaaggctacaccagctgtcttggctgaccaaggacagccagaatatcatgctcactgtgagggacgagcttacttgccgcatcggttagggccgacccctccgatgttgaatgtccctgaacattttcgccgtccatttaacatcggattattcagagggacaatcgacataaccctggtacttttcgatgatgaatctgtagattctgccccggtcatatgggatcattttaatgcatccagattgagcagcttcagagaaaaggctttgttgtttggtttgattctagaaaagaaagccactgggaattgggtattggactctattagtcatttcaagtaattatcacaagtgttgaggtgatgggcagactatgaaaaaaactaacagggttcaaacactcttgatcgaggtacccagttatatttgttacaacaatgctcaggatccagatccctccgattgctatcattctggtaagtctcctcacactcgacctgtccggtgcaaggaggacaaccacacaaagaatccctctccttaatgattcgtgggatttgttctcgagctatggcgacattcccgaagaacttgtcgtataccagaactacagccacaattcctccgagttaccccctcctggcttcgagagatggtacataaaccgaagagtggcagacacttccataccgtgcaggggcccctgtctagtgccctacatccttcatggcctcaatgacacaactgtctctcgacggggaggaggatggcgaaggtccggaatgaagtacccaacccacgctgtcaggctaggcccttcaacagacgacgagagagttgaggaagacatcggctacgtcaatgtctccgcactatcctgcacagggtcgcccgttgagatggcgataccaacaatccccgactgcaccagtgctatccatccacgatccgaggttactgtgcccgtcaagctcgatgtcatgagacgaaatcccaactaccctcccattagagcgtggtcgtgcatcggacagaaaatcaccaaccgatgtgattgggcactcttcggcgagaacctcatatatactcaagttgaagctagctctctagcattcaagcacacaagagcctctcttttgaacgaatccaacgggatagacgctgaaggacgtgcagttccctatatcctcggggatatcgaacccgggtactgccgaaccctattcaacacatgggtctctagtgagatcgtgtcatgcacgcccatcgaacttgtcctagttgacctgaaccctttgtccccgggacatggcggatatgctgtattgctgccaaacggagacaaagtggatgtacacgacaagcatgcatgggatggggacaacaaaatgtggagatgggtgtacgagaagaaagatccctgtgcgttcgagctggtatccagggaagtgtgtcttttctcactgagtaggggtagtagactgagaggagcaacccctccccaaggagagctcctcacctgcccgcattcgggaaaggcatttgacctgaagggggcccgaaggattacacccatttcatgcaaaatcgacatggaatatgacttgctgtcactaccaaccggagtcatcctaggcctccacctatcagaactcgggacctcctttggcaacctctcaatgagtcttgaaatgtatgaacctgccacaactctgacccctgagcaaatcaacttctcgcttaaagagctgggaagctggaccgaggctcaactgaagagcctgtctcactcaatctgcctctccacattctccatatgggaactatcggttgggatgatcgatctaaaccctaccagggcagcaagggccttgctccatgatgataacatactggcaacattcgagaacggtcacttttccatcgtcagatgtcgtccggaaatagttcaagtcccttcgcatcctcgagcatgtcacatggatctccgcccttatgacaagcaatcacgggcatcaaccctggtggttccccttgacaacagcactgccctcctggtccccgacaacatcgtggttgaaggagtagaggccagtctatgcaaccactccgttgccatcacgctgtcgaagaacagaactcactcatacagcctctatccccagggtcgtcctgtgcttcgacagaaaggtgccgtggagctcccgacgatagggcccctccagttacatcctgccactcgagtggacctttatacactgaaagagttccaggaggaccgaatagcgcgcagtcgagtcacagacatcaaggctgccgttgacgatctgcgtgcgaagtggcgtaaaggcaaatttgaggcggacaccacgggagggggactttggtcggcgattgtgggagtcttcagttctctcggggggttcttcatgaggcccttgattgctctcgcggcgatagtgacctcaatcatcatcctgtatatccttctgcgtgtactgtgtgctgcctcatgttcgacacaccgaagagtaaggcaggactcttggtaagcggccgccagatgacgcatgagggtcagatcagatttacagcgtaagtgtgatatttaggattataaaggttccttcattttaatttgttacagactgtatgaaaaaaactcatcaacagccatcatggatgttaacgattttgagttgcatgaggactttgcattgtctgaagatgactttgtcacttcagaatttctcaatccggaagaccaaatgacatacctgaatcatgccgattataatttgaattctcccttaatcagcgatgatattgatttcctgatcaagaaatataatcatgagcaaattccgaaaatgtgggatgtcaagaattgggagggagtgttagagatgttgacagcctgtcaagccagtccaattttatctagcactatgcataagtgggtgggaaagtggctcatgtctgatgatcatgacgcaagccaaggcttcagttttcttcatgaagtggacaaagaagctgatctgacgtttgaggtggtggagacattcattagaggatggggaggtcgagaattgcagtacaagaggaaagacacatttccggactcctttagagttgcagcctcattgtgtcaaaaattccttgatttgcacaaactcactctgataatgaattcagtctctgaagtcgaacttaccaacctagcaaagaattttaaaggaaaaaacaggaaagcaaaaagcggaaatctgataaccagattgagggttcccagtttaggtcctgcttttgtgactcagggatgggtgtacatgaagaagttggaaatgattatggatcggaattttttgttgatgttgaaagacgttatcatcgggaggatgcagacgatcctgtccatgatctcaagagatgataatctcttctccgagtctgatatctttactgtattaaagatataccggataggggataagatattagaaaggcaagggacaaagggttacgacttgatcaaaatgattgagcctatttgtaacttaaagatgatgaatctggcacgtaaatatcgtcctctcatccctacatttcctcattttgaaaaacatattgctgactctgttaaggaaggatcgaaaatagacaaagggattgagtttatatatgatcacattatgtcaatccctggtgtggacttgaccttagttatttacggatcatttcggcactggggtcatccttttatcaactactatgagggcttagagaagctacacaagcaggttacaatgcccaagactattgacagagaatatgcagaatgtcttgctagtgatctggcaagaatcgttcttcagcaacaattcaatgaacataagaaatggtttgttgatgtagataaagtcccacaatcccatcctttcaaaagccatatgaaagagaatacttggcctactgcagcccaagttcaggattacggcgatcgctggcatcagctcccactcatcaaatgcttcgaaatcccagatttgttagatccatcgatcatctactcagacaaaagtcattccatgaaccggtctgaagtactacgacatgtaagacttacacctcatgtgcccattccaagcaggaaagtattgcagacaatgttggagactaaggcaacagactggaaagagtttttaaagaaaattgacgaagaggggttagaggatgatgatcttgtcataggactcaaagggaaagagagagaattaaaaattgcgggaagattcttttctttgatgtcctggaagctcagagagtattttgtcatcactgagtatttgattaagacgcactttgtcccgatgtttaaagggttgaccatggcggatgacttgacagcggtgataaagaagatgatggacacatcttcaggacaaggcttagataattatgaatccatttgtatagccaaccatattgactatgagaagtggaacaatcatcaaagaaaagagtcgaacgggcccgtgttcaaggtgatgggtcaattcttgggatatccacgtctgattgagagaactcatgaattttttgagaagagtctgatatattacaatggacgaccagatctgatgcgggttcgaggaaattctctagtcaacgcctcatctttaaatgtctgctgggagggtcaagctgggggattagaaggactgcgacagaagggatggagtattctaaatttgcttgtcattcagagagaagcaaaaataaggaacaccgccgtgaaagtgctagctcaaggtgacaatcaggtgatatgtactcagtataaaacgaagaaatcccggaatgatattgagcttaaggcagctctaacacagatggtatctaataatgagatgattatgtctgcgattaaatcaggcaccgagaaactgggtcttttgattaatgatgatgagacaatgcaatctgctgattacctcaattacgggaaggttcccattttcagaggagtaatcagaggccttgagacaaaaagatggtcacgcgtgacctgtgtgacaaatgatcagattccaacgtgtgcgaacattatgagctctgtgtcaactaatgcattaactgtagcccattttgccgagaatccagtcaatgccatcattcagtataactactttggaacatttgcaaggctactgctgatgatgcatgaccccgctctgaggatctctctgtatgaagtccaatcaaaaattccaggacttcacagtttgacatttaaatattctatgttgtatctggatccttcgataggaggagtctccggaatgtcactctcgagattcctcataagatcatttccagatccagtgacagaaagtttggcgttctggaaatttatccactctcatgcaagaagcgattcattaaaggagatatgtgcagtttttggaaatcctgaaattgcaagatttcggctaactcatgtcgataaattggtggaagacccaacctcattgaacatagctatgggaatgagtcctgctaatctattaaagacagaggtaaaaaaatgtctactggaatcaaggcagagcatcaagaaccagattgtaagagatgctactatttacctacaccatgaggaagacaaacttcgtagtttcttatggtccataacaccactgttccctcggttcttgagtgaattcaaatctgggacattcatcggagtagcagatggcctgatcagcttatttcagaactctaggactattcgaaattcttttaaaaagcgttatcacagggaacttgatgatttaataatcaagagcgaagtttcctcacttatgcatttgggtaagctacatttgaggcgaggctcagttcgtatgtggacttgctcttctactcaggctgatcttctccgattccggtcatggggaagatctgttataggaaccacagtccctcatcccttagagatgttaggacaacattttaaaaaggagactccttgcagtgcttgcaacatatccggattagactatgtatctgtccactgtccgaatgggattcatgacgtttttgaatcacgtggtccactccctgcatatttgggttctaaaacatccgaatcaacttcgatcttgcagccgtgggagagagagagtaaagtaccgttgattaagcgtgccacaaggcttcgtgatgcaatttcatggtttgtgtctcccgactctaacttggcctcaactatccttaagaacataaatgcattaacaggagaagaatggtcaaagaagcagcatggatttaaaaggacgggatcggcgttacacaggttctccacatccaggatgagtcatggtggttttgcttctcagagtacggctgccttgactagattgatggcaactactgacactatgagagatctgggagaacagaactatgatttcctgtttcaggcgacattattgtatgctcaaataaccacaactgtagtcaggaatggatcatttcatagctgcacggaccattaccatataacctgcaaatcttgtctgagggccattgatgagattaccttggattcagcgatggaatatagccctccagatgtatcatcagttttacaatcttggaggaatggagaaggctcttggggacatgaagtgaaacaaatatacccagttgaaggtgactggaggggactatctcctgttgaacaatcttatcaagtcggacgctgtatcgggtttctgttcggtgatctggcgtatagaaaatcatcccatgcagatgatagctccatgtttccgttatctatacaaaacaaagtcagaggaagaggctttttaaaagggcttatggatgggttaatgagagccagttgttgccaggtgatccatcgtcgaagcttagcccatctgaagagaccggctaatgcagtctatggagggctgatttatttgatagacaaattgagtgcatctgccccttttctttcactgacgagacatggacctttaagggaagaattagaaactgttccacataagataccgacttcttatcctacgagcaaccgagatatgggggtgatagttcgtaattattttaaatatcagtgcagactggtagaaaaaggtcggtacaagacacattatcctcaattgtggcttttctcagatgtgctgtccattgatttcttaggacccctgtctatatcttcaactctattgggtattctgtataaacagacgttatcttctcgagacaaaaatgagttgagagaactcgctaacttgtcttcattgttgagatcaggagaaggatgggaagatatccatgtcaaattcttctctaaggacactttactctgccctgaagagatccgacatgcgtgcaaatttgggattgctaaggaatccgctgttttaagctattatcctccttggtctcaagagtcttatggaggcatcacctcgatccccgtatatttttcgaccaggaagtatcccaaaattttagatgtccctcctcgggttcaaaacccattggtctcgggtctacgattggggcaactccctactggagcacattataagattaggagcattgtaaagaacaagaaccttcgttatagagatttccttagttgtggggatggatctggggggatgaccgcggcactattgagagaaaacagacaaagtaggggaatcttcaacagcctgttagagttagccggatctcttatgagaggagcatctccagagcctccaagtgcactggagacgctcgggcaagaacgatctaggtgtgtgaatggaagcacatgttgggagtactcatctgacctaagccaaaaagagacatgggattacttcttaagattgaagagaggcctgggtttgaccgtggacttaatcaccatggacatggaggtcagagaccctaatacaagtttgatgatagaaaagaacctcaaagtttatctgcatcagatattagaaccaactggtgtcttaatatataaaacatacgggacccatattgcgacacaaacagataatatcctgacgataatcggtcctttctttgagacggttgacctagtccagtccgaatacagcagctcacaaacgtccgaggtctattttgtaggacgaggcttgcgctctcatgttgacgaaccctgggtggactggccatccttaatggacaattggagatccatttatgcttttcatgatcctactacagaatttatcagagcaaaaaaagtctgtgaaattgacagtcttataggcattccggctcaattcattccagacccatttgtaaatctcgagaccatgctacagatagttggtgttccaacaggagtttcgcatgccgcagctctattatcatcacaatatccaaatcaattggtcacaacgtcaatattttatatgacactcgtgtcttattataatgtaaaccatattcgaagaagccccaagcctttctctcctccgtctgatggagtctcacagaacattggttcagccatagtcggactaagtttttgggtgagtttgatggagaatgatctcggattatacaaacaggctctaggtgcaataaagacgtcattccctattagatggtcctctgtccagaccaaggatgggtttacacaagaatggagaactaaaggaaacggaattcctaaagattgtcgtctctcagactctttggctcagataggaaactggatcagagcgatggaattggttaggaacaaaacgaggcaatcaggattttctgaaaccctatttgatcaattctgcggacttgcagaccatcacctcaaatggcggaagttgggaaacagaacaggaattattgattggctaaataatagaatttcatccattgacaaatccatcttggtgaccaaaagtgatctgcatgacgagaactcatggagggagtgaagatgtattcttccacctctcattgggtgatacccatatatgaaaaaaactataagtactttaaactctctttgttttttaatgtatatctggttttgttgtttccgt

SEQ ID NO: 16 - RNA, 11719 nt, Artificial
  Chimeric virus containing RNA from Maraba and Farmington viruses
acgaagacaaacaaaccauugauagaauuaagaggcucaugaaaauccuuaacagcguucaaaaugucuguuacagucaagagagucauugaugauucacucaucacccccaaauugccugcgaaugaggacccuguggaguacccugcugauuauuucaaaaagucccgugauauuccgguguacauaaacacgaccaaaaguuugucugauuugcggggcuauguuuaucaaggccuaaagucaggcaacaucucuauaauucaugucaacaguuaucuguaugcagcauuaaaagagaucagaggaaaauuggacagagauuggaucaccuuugguauccaaaucggaaaaacaggagauagcguggggauauucgauuuacugacccuaaaaccucuagaugguguuuuaccagauggggugucugaugcuacucgaacuagcucagacgaugcauggcuuccacuguaucuauugggguuauacagaguuggucgaacacagaugccagaauacaggaagaagcugauggauggucugauuaaucaauguaagaugaucaaugagcaguuugaaccacuguugccagaaggaagagaugucuuugaugucuggggaaaugacagcaauuacacaaagauuguggccgcuguagauauguucuuccauauguucaaaaagcaugagaaggccucuuucagguauggcacaauagugucaagauuuaaggauugugcagcauuggcuacauuuggucaucuguguaagaucacugguauguccacugaagaugugacaacuuggauucuaaacagggagguggcugaugagaugguucaaaugauguacccaggacaggagauagauaaggcugauucuuacaugccuuaucuaaucgacuuaggucuguccucaaaaucuccauauucaucaguuaaaaauccagcuuuccauuuuuggggucaauugaccgcauuguuacugagaucaaccagagccagaaaugcacgucagccggaugacaucgaguauacaucccugaccacugcugggcuguuguaugcauaugccguugguucgucugcagaccuggcucaacaauucuacguuggggacaacaaguaugugccagaaacuggagauggaggauuaaccaccaaugcaccgccacaagggcgagauguggucgaguggcuuaguugguuugaagaucaaaacagaaaaccuaccccagacaugcucauguaugcuaagagagcugucagugcuuuacaaggauugagggagaagacgauuggcaaguacgccaagucagaguuugacaaaugacaacucacucaccauauguauuacuaccuuugcuucauaugaaaaaaacuaacagcgaucauggaucagcuaucaaaggucaaggaauuccuuaagacuuacgcgcaguuggaucaagcaguacaagagauggaugacauugagucucagagagaggaaaagacuaauuuugauuuguuucaggaagaaggauuggagauuaaggagaagccuuccuauuaucgggcagaugaagaagagauugauucagaugaagacagcguggaugaugcacaagacuuagggauacguacaucaacaagucccaucgagggguauguggaugaggagcaggaugauuaugaggaugaggaagugaacgugguguuuacaucggacuggaaacagccugagcuggaauccgacggggaugggaaaacucuccgauugacgauaccagauggauugacuggggagcagaagucgcaauggcuugccacgauuaaggcaguuguucagagugcuaaauauuggaacaucucagaauguucauuugagaguuaugagcaagggguuuugauuagagagagacaaaugacuccugaugucuacaaagucacuccuguuuuaaaugcuccaccgguucaaaugacagcuaaucaagauguuuggucucucagcagcacuccauuuacauuuuugcccaagaaacaaggugugacuccauugaccauguccuuagaagaacucuucaacacccgaggugaauucauaucucugggaggaaacgggaaaaugagucaccgggaggccaucauucuaggguugagacacaagaagcucuauaaucaagccagacuaaaguauaacuuagcuugaauaugaaaaaaacuaacagauaucaaaagauaucucuaacucaguccauuguguucaguucaaucaugagcucucucaagaaaauuuuggguauuaaagggaaagggaagaaaucuaagaaauuagguauggcucccccacccuaugaagaagagacuccaauggaauauucuccaagugcaccuuaugauaagucauuguuuggagucgaagauauggauuuccaugaucaacgucaacuccgauaugagaaauuucacuucucauugaagaugacugugagaucaaacaaaccauuucgaaauuaugaugacguugcagcagcgguguccaauugggaucauauguacaucggcauggcaggaaaacguccuuuuuauaagauauuagcauucauggguucuacucuauugaaggcuacaccagcugucuuggcugaccaaggacagccagaauaucaugcucacugugagggacgagcuuacuugccgcaucgguuagggccgaccccuccgauguugaaugucccugaacauuuucgccguccauuuaacaucggauuauucagagggacaaucgacauaacccugguacuuuucgaugaugaaucuguagauucugccccggucauaugggaucauuuuaaugcauccagauugagcagcuucagagaaaaggcuuuguuguuugguuugauucuagaaaagaaagccacugggaauuggguauuggacucuauuagucauuucaaguaauuaucacaaguguugaggugaugggcagacuaugaaaaaaacuaacaggguucaaacacucuugaucgagguacccaguuauauuuguuacaacaaugcucaggauccagaucccuccgauugcuaucauucugguaagucuccucacacucgaccuguccggugcaaggaggacaaccacacaaagaaucccucuccuuaaugauucgugggauuuguucucgagcuauggcgacauucccgaagaacuugucguauaccagaacuacagccacaauuccuccgaguuacccccuccuggcuucgagagaugguacauaaaccgaagaguggcagacacuuccauaccgugcaggggccccugucuagugcccuacauccuucauggccucaaugacacaacugucucucgacggggaggaggauggcgaagguccggaaugaaguacccaacccacgcugucaggcuaggcccuucaacagacgacgagagaguugaggaagacaucggcuacgucaaugucuccgcacuauccugcacagggucgcccguugagauggcgauaccaacaauccccgacugcaccagugcuauccauccacgauccgagguuacugugcccgucaagcucgaugucaugagacgaaaucccaacuacccucccauuagagcguggucgugcaucggacagaaaaucaccaaccgaugugauugggcacucuucggcgagaaccucauauauacucaaguugaagcuagcucucuagcauucaagcacacaagagccucucuuuugaacgaauccaacgggauagacgcugaaggacgugcaguucccuauauccucggggauaucgaacccggguacugccgaacccuauucaacacaugggucucuagugagaucgugucaugcacgcccaucgaacuuguccuaguugaccugaacccuuuguccccgggacauggcggauaugcuguauugcugccaaacggagacaaaguggauguacacgacaagcaugcaugggauggggacaacaaaauguggagauggguguacgagaagaaagaucccugugcguucgagcugguauccagggaagugugucuuuucucacugaguagggguaguagacugagaggagcaaccccuccccaaggagagcuccucaccugcccgcauucgggaaaggcauuugaccugaagggggcccgaaggauuacacccauuucaugcaaaaucgacauggaauaugacuugcugucacuaccaaccggagucauccuaggccuccaccuaucagaacucgggaccuccuuuggcaaccucucaaugagucuugaaauguaugaaccugccacaacucugaccccugagcaaaucaacuucucgcuuaaagagcugggaagcuggaccgaggcucaacugaagagccugucucacucaaucugccucuccacauucuccauaugggaacuaucgguugggaugaucgaucuaaacccuaccagggcagcaagggccuugcuccaugaugauaacauacuggcaacauucgagaacggucacuuuuccaucgucagaugucguccggaaauaguucaagucccuucgcauccucgagcaugucacauggaucuccgcccuuaugacaagcaaucacgggcaucaacccuggugguuccccuugacaacagcacugcccuccugguccccgacaacaucgugguugaaggaguagaggccagucuaugcaaccacuccguugccaucacgcugucgaagaacagaacucacucauacagccucuauccccagggucguccugugcuucgacagaaaggugccguggagcucccgacgauagggccccuccaguuacauccugccacucgaguggaccuuuauacacugaaagaguuccaggaggaccgaauagcgcgcagucgagucacagacaucaaggcugccguugacgaucugcgugcgaaguggcguaaaggcaaauuugaggcggacaccacgggagggggacuuuggucggcgauugugggagucuucaguucucucgggggguucuucaugaggcccuugauugcucucgcggcgauagugaccucaaucaucauccuguauauccuucugcguguacugugugcugccucauguucgacacaccgaagaguaaggcaggacucuugguaagcggccgccagaugacgcaugagggucagaucagauuuacagcguaagugugauauuuaggauuauaaagguuccuucauuuuaauuuguuacagacuguaugaaaaaaacucaucaacagccaucauggauguuaacgauuuugaguugcaugaggacuuugcauugucugaagaugacuuugucacuucagaauuucucaauccggaagaccaaaugacauaccugaaucaugccgauuauaauuugaauucucccuuaaucagcgaugauauugauuuccugaucaagaaauauaaucaugagcaaauuccgaaaaugugggaugucaagaauugggagggaguguuagagauguugacagccugucaagccaguccaauuuuaucuagcacuaugcauaagugggugggaaaguggcucaugucugaugaucaugacgcaagccaaggcuucaguuuucuucaugaaguggacaaagaagcugaucugacguuugaggugguggagacauucauuagaggauggggaggucgagaauugcaguacaagaggaaagacacauuuccggacuccuuuagaguugcagccucauugugucaaaaauuccuugauuugcacaaacucacucugauaaugaauucagucucugaagucgaacuuaccaaccuagcaaagaauuuuaaaggaaaaaacaggaaagcaaaaagcggaaaucugauaaccagauugaggguucccaguuuagguccugcuuuugugacucagggauggguguacaugaagaaguuggaaaugauuauggaucggaauuuuuuguugauguugaaagacguuaucaucgggaggaugcagacgauccuguccaugaucucaagagaugauaaucucuucuccgagucugauaucuuuacuguauuaaagauauaccggauaggggauaagauauuagaaaggcaagggacaaaggguuacgacuugaucaaaaugauugagccuauuuguaacuuaaagaugaugaaucuggcacguaaauaucguccucucaucccuacauuuccucauuuugaaaaacauauugcugacucuguuaaggaaggaucgaaaauagacaaagggauugaguuuauauaugaucacauuaugucaaucccugguguggacuugaccuuaguuauuuacggaucauuucggcacuggggucauccuuuuaucaacuacuaugagggcuuagagaagcuacacaagcagguuacaaugcccaagacuauugacagagaauaugcagaaugucuugcuagugaucuggcaagaaucguucuucagcaacaauucaaugaacauaagaaaugguuuguugauguagauaaagucccacaaucccauccuuucaaaagccauaugaaagagaauacuuggccuacugcagcccaaguucaggauuacggcgaucgcuggcaucagcucccacucaucaaaugcuucgaaaucccagauuuguuagauccaucgaucaucuacucagacaaaagucauuccaugaaccggucugaaguacuacgacauguaagacuuacaccucaugugcccauuccaagcaggaaaguauugcagacaauguuggagacuaaggcaacagacuggaaagaguuuuuaaagaaaauugacgaagagggguuagaggaugaugaucuugucauaggacucaaagggaaagagagagaauuaaaaauugcgggaagauucuuuucuuugauguccuggaagcucagagaguauuuugucaucacugaguauuugauuaagacgcacuuugucccgauguuuaaaggguugaccauggcggaugacuugacagcggugauaaagaagaugauggacacaucuucaggacaaggcuuagauaauuaugaauccauuuguauagccaaccauauugacuaugagaaguggaacaaucaucaaagaaaagagucgaacgggcccguguucaaggugaugggucaauucuugggauauccacgucugauugagagaacucaugaauuuuuugagaagagucugauauauuacaauggacgaccagaucugaugcggguucgaggaaauucucuagucaacgccucaucuuuaaaugucugcugggagggucaagcugggggauuagaaggacugcgacagaagggauggaguauucuaaauuugcuugucauucagagagaagcaaaaauaaggaacaccgccgugaaagugcuagcucaaggugacaaucaggugauauguacucaguauaaaacgaagaaaucccggaaugauauugagcuuaaggcagcucuaacacagaugguaucuaauaaugagaugauuaugucugcgauuaaaucaggcaccgagaaacugggucuuuugauuaaugaugaugagacaaugcaaucugcugauuaccucaauuacgggaagguucccauuuucagaggaguaaucagaggccuugagacaaaaagauggucacgcgugaccugugugacaaaugaucagauuccaacgugugcgaacauuaugagcucugugucaacuaaugcauuaacuguagcccauuuugccgagaauccagucaaugccaucauucaguauaacuacuuuggaacauuugcaaggcuacugcugaugaugcaugaccccgcucugaggaucucucuguaugaaguccaaucaaaaauuccaggacuucacaguuugacauuuaaauauucuauguuguaucuggauccuucgauaggaggagucuccggaaugucacucucgagauuccucauaagaucauuuccagauccagugacagaaaguuuggcguucuggaaauuuauccacucucaugcaagaagcgauucauuaaaggagauaugugcaguuuuuggaaauccugaaauugcaagauuucggcuaacucaugucgauaaauugguggaagacccaaccucauugaacauagcuaugggaaugaguccugcuaaucuauuaaagacagagguaaaaaaaugucuacuggaaucaaggcagagcaucaagaaccagauuguaagagaugcuacuauuuaccuacaccaugaggaagacaaacuucguaguuucuuaugguccauaacaccacuguucccucgguucuugagugaauucaaaucugggacauucaucggaguagcagauggccugaucagcuuauuucagaacucuaggacuauucgaaauucuuuuaaaaagcguuaucacagggaacuugaugauuuaauaaucaagagcgaaguuuccucacuuaugcauuuggguaagcuacauuugaggcgaggcucaguucguauguggacuugcucuucuacucaggcugaucuucuccgauuccggucauggggaagaucuguuauaggaaccacagucccucaucccuuagagauguuaggacaacauuuuaaaaaggagacuccuugcagugcuugcaacauauccggauuagacuauguaucuguccacuguccgaaugggauucaugacguuuuugaaucacgugguccacucccugcauauuuggguucuaaaacauccgaaucaacuucgaucuugcagccgugggagagagagaguaaaguaccguugauuaagcgugccacaaggcuucgugaugcaauuucaugguuugugucucccgacucuaacuuggccucaacuauccuuaagaacauaaaugcauuaacaggagaagaauggucaaagaagcagcauggauuuaaaaggacgggaucggcguuacacagguucuccacauccaggaugagucauggugguuuugcuucucagaguacggcugccuugacuagauugauggcaacuacugacacuaugagagaucugggagaacagaacuaugauuuccuguuucaggcgacauuauuguaugcucaaauaaccacaacuguagucaggaauggaucauuucauagcugcacggaccauuaccauauaaccugcaaaucuugucugagggccauugaugagauuaccuuggauucagcgauggaauauagcccuccagauguaucaucaguuuuacaaucuuggaggaauggagaaggcucuuggggacaugaagugaaacaaauauacccaguugaaggugacuggaggggacuaucuccuguugaacaaucuuaucaagucggacgcuguaucggguuucuguucggugaucuggcguauagaaaaucaucccaugcagaugauagcuccauguuuccguuaucuauacaaaacaaagucagaggaagaggcuuuuuaaaagggcuuauggauggguuaaugagagccaguuguugccaggugauccaucgucgaagcuuagcccaucugaagagaccggcuaaugcagucuauggagggcugauuuauuugauagacaaauugagugcaucugccccuuuucuuucacugacgagacauggaccuuuaagggaagaauuagaaacuguuccacauaagauaccgacuucuuauccuacgagcaaccgagauaugggggugauaguucguaauuauuuuaaauaucagugcagacugguagaaaaaggucgguacaagacacauuauccucaauuguggcuuuucucagaugugcuguccauugauuucuuaggaccccugucuauaucuucaacucuauuggguauucuguauaaacagacguuaucuucucgagacaaaaaugaguugagagaacucgcuaacuugucuucauuguugagaucaggagaaggaugggaagauauccaugucaaauucuucucuaaggacacuuuacucugcccugaagagauccgacaugcgugcaaauuugggauugcuaaggaauccgcuguuuuaagcuauuauccuccuuggucucaagagucuuauggaggcaucaccucgauccccguauauuuuucgaccaggaaguaucccaaaauuuuagaugucccuccucggguucaaaacccauuggucucgggucuacgauuggggcaacucccuacuggagcacauuauaagauuaggagcauuguaaagaacaagaaccuucguuauagagauuuccuuaguuguggggauggaucuggggggaugaccgcggcacuauugagagaaaacagacaaaguaggggaaucuucaacagccuguuagaguuagccggaucucuuaugagaggagcaucuccagagccuccaagugcacuggagacgcucgggcaagaacgaucuaggugugugaauggaagcacauguugggaguacucaucugaccuaagccaaaaagagacaugggauuacuucuuaagauugaagagaggccuggguuugaccguggacuuaaucaccauggacauggaggucagagacccuaauacaaguuugaugauagaaaagaaccucaaaguuuaucugcaucagauauuagaaccaacuggugucuuaauauauaaaacauacgggacccauauugcgacacaaacagauaauauccugacgauaaucgguccuuucuuugagacgguugaccuaguccaguccgaauacagcagcucacaaacguccgaggucuauuuuguaggacgaggcuugcgcucucauguugacgaacccuggguggacuggccauccuuaauggacaauuggagauccauuuaugcuuuucaugauccuacuacagaauuuaucagagcaaaaaaagucugugaaauugacagucuuauaggcauuccggcucaauucauuccagacccauuuguaaaucucgagaccaugcuacagauaguugguguuccaacaggaguuucgcaugccgcagcucuauuaucaucacaauauccaaaucaauuggucacaacgucaauauuuuauaugacacucgugucuuauuauaauguaaaccauauucgaagaagccccaagccuuucucuccuccgucugauggagucucacagaacauugguucagccauagucggacuaaguuuuugggugaguuugauggagaaugaucucggauuauacaaacaggcucuaggugcaauaaagacgucauucccuauuagaugguccucuguccagaccaaggauggguuuacacaagaauggagaacuaaaggaaacggaauuccuaaagauugucgucucucagacucuuuggcucagauaggaaacuggaucagagcgauggaauugguuaggaacaaaacgaggcaaucaggauuuucugaaacccuauuugaucaauucugcggacuugcagaccaucaccucaaauggcggaaguugggaaacagaacaggaauuauugauuggcuaaauaauagaauuucauccauugacaaauccaucuuggugaccaaaagugaucugcaugacgagaacucauggagggagugaagauguauucuuccaccucucauugggugauacccauauaugaaaaaaacuauaaguacuuuaaacucucuuuguuuuuuaauguauaucugguuuuguuguuuccgu

SEQ ID NO: 17 - protein, 704 aa, Farmington virus
MLRIQIPPIAIILVSLLTLDLSGARRTTTQRIPLLNDSWDLFSSYGDIPEELVVYQNYSHNSSELPPPGFERWYINRRVADTSIPCRGPCLVPYILHGLNDTTVSRRGGGWRRSGMKYPTHAVRLGPSTDDERVEEDIGYVNVSALSCTGSPVEMAIPTIPDCTSAIHPRSEVTVPVKLDVMRRNPNYPPIRAWSCIGQKITNRCDWALFGENLIYTQVEASSLAFKHTRASLLNESNGIDAEGRAVPYILGDIEPGYCRTLFNTWVSSEIVSCTPIELVLVDLNPLSPGHGGYAVLLPNGDKVDVHDKHAWDGDNKMWRWVYEKKDPCAFELVSREVCLFSLSRGSRLRGATPPQGELLTCPHSGKAFDLKGARRITPISCKIDMEYDLLSLPTGVILGLHLSELGTSFGNLSMSLEMYEPATTLTPEQINFSLKELGSWTEAQLKSLSHSICLSTFSIWELSVGMIDLNPTRAARALLHDDNILATFENGHFSIVRCRPEIVQVPSHPRACHMDLRPYDKQSRASTLVVPLDNSTALLVPDNIVVEGVEASLCNHSVAITLSKNRTHSYSLYPQGRPVLRQKGAVELPTIGPLQLHPATRVDLYTLKEFQEDRIARSRVTDIKAAVDDLRAKWRKGKFEADTTGGGLWSAIVGVFSSLGGFFMRPLIALAAIVTSIIILYILLRVLCAASCSTHRRVRQDSW

SEQ ID NO: 18 - DNA, 12695 bp, Artificial
  DNA encoded by chimeric virus that contains RNA from Maraba and Farmington viruses
ttacgacgcataagctgagaaacataagagactatgttcatagtcaccctgtattcattattgacttttatgacctattattcgtgaggtcatatgtgaggtaatgtcatctgcttatgcgtttgcttataagataaaacgatagacccttcacgggtaaatccttctccttgcagttctcgccaagtacctccaaagtcagaacgatggctcgtccgctagctgctgcgcaacatctcataaccgagcgtcattcccttcaggcgactctgtcgcgggcgtccaagaccagagccgaggaattcgtcaaagatttctaccttcaagagcagtattctgtcccgaccatcccgacggacgacattgcccagtctgggcccatgctgcttcaggccatcctgagcgaggaatacacaaaggccactgacatagcccaatccatcctctggaacactcccacacccaacgggctcctcagagagcatctagatgccgatgggggaggctcattcacagcgctgcccgcgtctgcaatcagacccagcgacgaggcgaatgcatgggccgctcgcatctccgactcagggttggggcctgtcttctatgcagccctcgctgcttacatcatcggctggtcaggaagaggagagactagccgcgtgcagcagaacataggtcagaaatggctgatgaacctgaacgcaatcttcggcaccacgatcacccatccaacaaccgtgcgtctgccaatcaacgtcgtcaacaacagcctcgcagtgaggaacggacttgctgccacactctggctatactaccgttcatcacctcagagtcaggacgcgttcttctatgggctcatccgtccctgttgcagtggatatctcggcctgctacatcgggtgcaggagattgatgagatggagccggacttcctcagtgacccccggatcatccaggtgaatgaggtctacagtgcactcagagccctggttcaactgggaaacgacttcaagaccgccgatgatgagcccatgcaggtctgggcgtgcaggggaatcaacaacggatatctgacatatctctcagaaactcctgcgaagaaaggagctgttgtgcttatgtttgcccaatgcatgctgaagggcgactctgaggcctggaacagctaccgcactgcaacctgggtgatgccctattgcgacaatgtggccctaggagcgatggcaggctacatccaagcccgccagaacaccagggcatatgaggtctcagcccagacaggtctcgacgtcaacatggccgcggtcaaggactttgaggccagttcaaaacccaaggctgctccaatctcgctgatcccacgccccgctgatgtcgcatcccgcacctctgagcgcccatctattcctgaggttgacagcgacgaagagctcggaggaatgtaaaccaataagcttcactgccggtagtttaggcatacacacgcagttccgttatccatcacacccgtcccttcttttatgctgctattatttcagttgctaagcttcctgatttgattaacaaaaaaccgtagacctcctacgtgaggtatagctagaaattggttctatcggttgagagtctttgtactattagccatggaggactatttgtctagcttagaggccgcgagagagctcgtccggacggagctggagcccaagcgtaacctcatagccagcttagagtccgacgatcccgatccggtaatagcgccagcggtaaaaccaaaacatcccaagccatgcctgagcactaaagaagaggatcatctcccctctcttcgcctactattcggcgcaaaacgagacacctcggtgggcgtagagcagactctccacaagcgtctctgcgcttgtctcgacggttacctgaccatgacgaagaaagaggccaatgcctttaaggccgcggctgaagcagcagcattagcagtcatggacattaagatggagcatcagcgccaggatctagaggatctgaccgctgctatccctaggatagaattcaaactcaatgccatcctggaaaacaacaaggagatagccaaggctgtaactgctgctaaggagatggagcgggagatgtcgtggggggaaagcgccgccagctcgctcaagtctgtcaccctagatgagtcgtttaggggccctgaagagctttcagagtcatttggcatccgatataaggtcagaacctggaatgagttcaagaaggcgctggaaaccagcattgtggacctgaggcctagccctgtttcatttagggaattacggactatgtggctgtctcttgacacctcctttaggctcattgggtttgccttcattcccacatgcgagcgcctggagaccaaagccaaatgcaaggagacaaggactctactcccccttgcagagtcgatcatgcgaagatgggacctgcgggatccaaccatcttggagaaagcctgcgtagtaatgatgatccgtgggaatgagattgcatcgctgaatcaggtaaaagatgttctcccgaccacaattcgtgggtggaagatcgcttattagtcactgctcccattagtcccactagacggcatacttccattccgccctttaattcccctgtcagacactcatgctccgaaatcactaaccatccttgtccaccaagcaatacgcatattcagtagcactgcatctcgccctccccctatcaagccccagcgctgcagatcttcaccacatatatacatgcatcaactacatgtgatttagaaaaaaccagacccttcacgggtaatagcctaactcacgaacgttcctctcgtttcgtatgataaggccttaagcattgtcgatacggtcgttatgcgtcggttctttttaggagagagcagtgcccctgcgagggactgggagtccgagcgacctcccccctatgctgttgaggtccctcaaagtcacgggataagagtcaccgggtacttccagtgcaacgagcgtccgaaatccaagaagaccctccacagcttcgccgtaaaactctgcgacgcaattaagccggttcgagcggatgctcccagcttgaagatagcaatatggacggctctagatctggccttcgtgaaacctcccaatggaactgtaacaatagatgcggcggtgaaagctacaccgctaatcgggaacacccagtacaccgtaggcgatgaaatcttccagatgctagggagaaggggtggcctgatcgtcatcaggaacttaccccatgattatcctcgaacgttgattgagttcgcctctcccgagccttgagcaccagggcatcggtccgcccgccctgtgatctcccgtagccgggctcagcgatcaagccggcccgggtcgggggggactggtgcaacacaaggggcggcagtggacgctgattaacaaaaaaccacctatatagacccctcacggtcttagactctgttgccagctgacaaccaacacacaagacatctctctgattcagccgacccgatcgattcctccccacccaattcctaccaacgcactcctcacaagctccaccggcgcgccatgttgagactttttctcttttgtttcttggccttaggagcccactccaaatttactatagtattccctcatcatcaaaaagggaattggaagaatgtgccttccacatatcattattgcccttctagttctgaccagaattggcataatgatttgactggagttagtcttcatgtgaaaattcccaaaagtcacaaagctatacaagcagatggctggatgtgccacgctgctaaatgggtgactacttgtgacttcagatggtacggacccaaatacatcacgcattccatacactctatgtcacccaccctagaacagtgcaagaccagtattgagcagacaaagcaaggagtttggattaatccaggctttccccctcaaagctgcggatatgctacagtgacggatgcagaggtggttgttgtacaagcaacacctcatcatgtgttggttgatgagtacacaggagaatggattgactcacaattggtggggggcaaatgttccaaggaggtttgtcaaacggttcacaactcgaccgtgtggcatgctgattacaagattacagggctgtgcgagtcaaatctggcatcagtggatatcaccttcttctctgaggatggtcaaaagacgtctttgggaaaaccgaacactggattcaggagtaatcactttgcttacgaaagtggagagaaggcatgccgtatgcagtactgcacacaatgggggatccgactaccttctggagtatggtttgaattagtggacaaagatctcttccaggcggcaaaattgcctgaatgtcctagaggatccagtatctcagctccttctcagacttctgtggatgttagtttgatacaagacgtagagaggatcttagattactctctatgccaggagacgtggagtaagatacgagccaagcttcctgtatctccagtagatctgagttatctcgccccaaaaaatccagggagcggaccggccttcactatcattaatggcactttgaaatatttcgaaacaagatacatcagagttgacataagtaatcccatcatccctcacatggtgggaacaatgagtggaaccacgactgagcgtgaattgtggaatgattggtatccatatgaagacgtagagattggtccaaatggggtgttgaaaactcccactggtttcaagtttccgctgtacatgattgggcacggaatgttggattccgatctccacaaatcctcccaggctcaagtcttcgaacatccacacgcaaaggacgctgcatcacagcttcctgatgatgagactttattttttggtgacacaggactatcaaaaaacccagtagagttagtagaaggctggttcagtagctggaagagcacattggcatcgttctttctgattataggcttgggggttgcattaatcttcatcattcgaattattgttgcgattcgctataaatacaaggggaggaagacccaaaaaatttacaatgatgtcgagatgagtcgattgggaaataaataaggcgcgccagaggactgcgattgttgagtggagccggcaaaccctaggcctattccgatttagaaaaaaccagacctctcacgaggtcttttctactagctgggttttcctcattctatccagagccgcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgccaaaccctaggcctattccgatttagaaaaaaccagacctctcacgaggtcttttctactagctgggttttcctcattctatccagagccatggccttcgacccgaactggcagagagaaggttatgaatgggatccgtcaagtgagggcagaccgaccgatgagaacgaagacgacagaggtcatcggccaaaaacgagacttcgtacattccttgcccgcacgttaaatagccctatccgagccctattctacacaatattcctaggaattcgagcggtttgggacgggttcaaaagactcctacctgtgaggaccgaaaagggttatgcgaggttttctgagtgcgtcacatatggaatgatcggatgtgatgagtgtgtaatagacccggtgagggttgtcattgagctgaccgagatgcagttaccgattaaaggcaaaggctctacgaggttgagagcaatgataactgaagaccttctcacggggatgcgcacagccgtgcctcagatcagagtgagatcgaagatcctagcagagcggttagggagagcaatcggccgagagaccttgccggcaatgatccatcatgagtgggcatttgtgatggggaagattctcactttcatggcagacaatgtgggtatgaacgctgacacggtcgagggcgttctatcactatcagaggtcacacggcgatgggatatcggcaactctgtgtccgcagtgttcaatcctgatggccttactatcagagtagaaaacacgggttacatcatgaccagagagactgcctgcatgatcggagacattcatgctcaatttgcaatccaatacctagctgcatacctagacgaggtgatcggcacaaggacgtctctctcacccgccgaactgacctctctcaaactatggggacttaacgtcctgaaactcctaggacggaacggttatgaggtgatcgcctgcatggagcccatagggtacgctgtcctgatgatgggaagagacaggagtcctgatccctatgtcaatgacacctatttaaacagcatcctctcagaattccctgtcgactctgacgctcgagcctgcgttgaagccctcttaactatctatatgagcttcggcacaccccataaagtctcggacgcattcggcctcttcagaatgttgggacatccgatggttgatggagctgacgggattgaaaagatgcgaaggttaagcaagaaggtcaagatcccagaccagtctacagcgatcgacctcggggctatcatggccgaactgtttgtgcggagtttcgtaaagaagcacaaaaggtggcccaactgctccatcaatctcccgccacgacaccccttccaccacgcccgcctatgtgggtatgtcccggctgaaacccatcccctaaacaacactgcatcctgggcggctgtggagttcaaccaggaattcgagccgccgagacagtacaaccttgcagacatcattgatgacaagtcgtgctctcccaacaagcatgagctatatggtgcttggatgaagtcaaaaacagctgggtggcaggaacaaaagaagctcatactccgatggttcactgagaccatggttaaaccttcggagctcctggaagagattgatgcacacggcttccgagaagaggataagttgattggattaacaccaaaggagagagagctgaaattaacaccaagaatgttctccttgatgacattcaagttcagaacctaccaagtcctcactgagagtatggtcgccgatgagatcctcccgcacttcccccagatcaccatgaccatgtccaaccacgaactcacaaagaggttgattagcagaacgagacctcaatctggaggagggcgtgatgttcacatcaccgtgaacatagatttccagaaatggaacacaaacatgagacacggactggtcaaacatgtcttcgagcgactggacaacctctttggcttcaccaacttaatcagacgaactcatgaatacttccaggaggcgaaatactatctggctgaagatggaactaatctgtcgttcgacaggaacggggagttaatagatggcccatacgtttacaccggatcatacggggggaacgaggggttacgacagaagccctggacaatagttaccgtgtgtggaatatacaaggtagctagagacctgaaaatcaaacatcagatcaccggtcagggagataatcaggtggtcaccctaatatttccggatcgagagttgccttcagatccggtggagaggagcaagtactgtagagacaagagcagtcagttcctgacacgtctcagtcaatatttcgctgaggttggtttgcccgtcaagactgaagagacatggatgtcatcacgtctctatgcttacggtaagcgcatgttcttagagggagttccacttaagatgtttctcaagaagataggcagagctttcgccctctcgaatgagtttgtcccgtccctcgaggaagatctggccagagtctggagtgccaccagcgcagcggtagagcttgacctaactccctacgtaggatatgtcctcgggtgctgcttgtctgcgcaggcgatcagaaatcacctcatctactcccctgttctggagggccctctgctggttaaggcctacgagcgtaagttcattaactacgacggaggaacaaagcggggggcgatgcccggcctacgtccaacctttgagagcctagtcaaaagtatctgctggaagccaaaggccatcggagggtggccggtattgatgttagaagatctcatcatcaaagggttccctgatccggcgactagcgccctggctcaattgaagtcaatggtgccatatacctctggtatcgaccgggagatcatactttcctgtctcaaccttcccttatcgtcggtggtatctccgtcaatgttgttaaaggacccggcggccatcaacaccatcacaaccccgtccgcgggcgacatcctgcaagaggtcgccagagactatgttaccgattacccactccaaaacccgcagctcagagcagtggtcaagaacgtgaagaccgagctagacacattggccagtgacttattcaaatgtgaacctttctttcctcctttaatgagcgatatcttctcggcatctctcccggcatatcaagacaggattgttcgcaagtgctccacgacttctacaatcaggagaaaagctgccgagaggggctccgactctctcctcaaccggatgaaaaggaatgagatcaataagatgatgttacatctttgggctacctggggaaggagccctctggccagattagacaccagatgtctcacaacctgcaccaagcaattagcccaacagtatcggaaccagtcttggggaaagcagatccatggagtctcagtcggccaccccttagaactgttcggtcgaataacacccagccatagatgcctacatgaggaggaccacggagatttcctgcaaaccttcgccagcgagcatgtgaaccaagtggacaccgacatcaccacaactctggggccgttctacccttacataggctcggagacgcgagaacgggcagtcaaggttcgaaaaggagtgaattacgtagttgagccgcttctgaaacccgcagttcgactactaagagccattaattggttcattcccgaggagtcagatgcgtcccatttgctgagcaatctattagcgtctgttaccgacatcaatcctcaagaccactactcatctaccgaagtaggggggggcaacgccgtccatcgctacagctgccgactatccgacaaattgagcagagtcaacaacttatatcagttgcatacttatttatctgtcacaacagagcggttgaccaagtacagtcgaggatcaaaaaacactgacgcacacttccagagcatgatgatttatgcacaaagccgtcatatagacctcatcttggagtctctgcacaccggagagatggtaccgttggagtgtcatcatcacattgagtgcaatcactgtatagaggatatacccgacgagccaatcacgggggacccggcttggactgaagtcaagtttccttcaagtcctcaggagccctttctttacatcaggcaacaagatctgccggtcaaagacaaactcgagcctgtgcctcgcatgaacatcgtccgtcttgccggattgggtccggaggcgattagtgagctagcgcactactttgttgcattccgagttatccgggcgtcagagacggatgtcgaccctaacgatgttctctcgtggacctggctgagccgaattgatcctgacaaattggttgagtatatcgtgcatgtgttcgcttcactggaatggcatcatgtattaatgtcaggcgtgagtgtgagcgtcagagatgcattctttaagatgctagtgtctaaaagaatctcagagactccgctaagttcattctattatctggccaacctgttcgttgaccctcagactcgcgaagcactaatgagctctaaatacgggttcagcccccccgccgagacagtccccaacgcaaatgccgccgcagccgaaataagaagatgctgtgcgaacagtgcgccgtcgatcttagaatcagcccttcacagccgtgaggttgtttggatgccaggaacgaacaattatggagacgttgtcatctggtctcattacattagattacggttcagcgaagttaaactagttgacattacacgatatcagcagtggtggagacagtctgagcgagacccctacgatttggtcccggacatgcaggttcttgagagcgacctagatacgctgatgaaacggataccgaggctcatgcgcaaggcgagacgtccccctcttcaggtaattcgagaggacctggatgtcgcagtcatcaatgctgatcatcccgctcactctgtgcttcagaacaaatacaggaaattgattttcagagagccgaagattatcacgggagctgtgtacaagtacctctccctaaaatcagagttgacagagttcacctcagcaatggtgatcggagacggaactggaggtatcaccgccgccatgatggccgatgggatagatgtgtggtatcagacgctcgtcaactatgaccacgtgacacaacagggattatccgtacaagccccggcagcattggatcttctgcgcggggcaccctctggtaggctcttgaatccgggaagattcgcatcatttgggtctgacctaactgaccctcgatttacagcctactttgatcaatatcccccgttcaaggtggacactctatggtctgacgcagagggcgacttttgggacaagccttccaagttgaatcaatactttgagaacatcattgctttgagacatcggttcgtgaagacaaatggacagcttgtcgtgaaggtgtatctgactcaagacactgctaccacaattgaagcattcagaaagaagctgtccccatgcgccatcatcgtgtctctcttctcgacggaaggctccacagaatgcttcgtcctaagcaatctcatcgcaccagacacccctgtcgaccttgagatggtggagaatatccctaaactaacatcccttgttccccagaggacgacagtgaaatgctattcccgacgagtagcgtgcatcagtaaaaggtggggacttttcagatctccgagcatagcccttgaagtccaaccgttccttcactacatcacaaaggtcatctcagacaaaggaacacaactgagtctcatggcggtagctgacacaatgatcaacagttacaagaaggctatctcaccccgagtgttcgatctacaccggcatagggccgcactgggtttcgggaggagatccttgcatctcatctgggggatgatcatctcaccaatcgcttaccagcattttgagaatccggccaagttgatggatgtcctggacatgttgaccaataacatctcagctttcttatcgatatcgtcgtcaggatttgacctgtcatttagtgtcagtgcagaccgagatgtccggattgacagcaaacttgtcagactcccgctattcgaaggatcagacctaaaattcatgaaaaccatcatgtctaccctcggatctgtgttcaaccaggtcgagccttttaaggggatcgccataaacccttctaaactaatgactgtcaagaggacacaggagttacgttacaacaacctaatttacactaaggatgccatcctattccccaatgaagcggcaaaaaacactgccccgcttcgagccaacatggtataccccgtccggggagatctattcgcccctaccgatcgcataccaatcatgactctagtcagcgatgagacaacacctcagcactctcctccagaggatgaggcataactgaatcctccctgaaggctcacatgtcccacgcgacgcaagatataacgacaagcaactcgccctattaactgtgattaataaaaaaccgattattcagttgcttgagggagtttcaatccgttcagtgtatgataggaagtttctgagatggtggggattagggggcacctagagtatgtttgttcgttttatgcgtcgt

SEQ ID NO: 19 - RNA, 12695 nt, Artificial
  Chimeric virus containing RNA from Maraba and Farmington viruses
uuacgacgcauaagcugagaaacauaagagacuauguucauagucacccuguauucauuauugacuuuuaugaccuauuauucgugaggucauaugugagguaaugucaucugcuuaugcguuugcuuauaagauaaaacgauagacccuucacggguaaauccuucuccuugcaguucucgccaaguaccuccaaagucagaacgauggcucguccgcuagcugcugcgcaacaucucauaaccgagcgucauucccuucaggcgacucugucgcgggcguccaagaccagagccgaggaauucgucaaagauuucuaccuucaagagcaguauucugucccgaccaucccgacggacgacauugcccagucugggcccaugcugcuucaggccauccugagcgaggaauacacaaaggccacugacauagcccaauccauccucuggaacacucccacacccaacgggcuccucagagagcaucuagaugccgaugggggaggcucauucacagcgcugcccgcgucugcaaucagacccagcgacgaggcgaaugcaugggccgcucgcaucuccgacucaggguuggggccugucuucuaugcagcccucgcugcuuacaucaucggcuggucaggaagaggagagacuagccgcgugcagcagaacauaggucagaaauggcugaugaaccugaacgcaaucuucggcaccacgaucacccauccaacaaccgugcgucugccaaucaacgucgucaacaacagccucgcagugaggaacggacuugcugccacacucuggcuauacuaccguucaucaccucagagucaggacgcguucuucuaugggcucauccgucccuguugcaguggauaucucggccugcuacaucgggugcaggagauugaugagauggagccggacuuccucagugacccccggaucauccaggugaaugaggucuacagugcacucagagcccugguucaacugggaaacgacuucaagaccgccgaugaugagcccaugcaggucugggcgugcaggggaaucaacaacggauaucugacauaucucucagaaacuccugcgaagaaaggagcuguugugcuuauguuugcccaaugcaugcugaagggcgacucugaggccuggaacagcuaccgcacugcaaccugggugaugcccuauugcgacaauguggcccuaggagcgauggcaggcuacauccaagcccgccagaacaccagggcauaugaggucucagcccagacaggucucgacgucaacauggccgcggucaaggacuuugaggccaguucaaaacccaaggcugcuccaaucucgcugaucccacgccccgcugaugucgcaucccgcaccucugagcgcccaucuauuccugagguugacagcgacgaagagcucggaggaauguaaaccaauaagcuucacugccgguaguuuaggcauacacacgcaguuccguuauccaucacacccgucccuucuuuuaugcugcuauuauuucaguugcuaagcuuccugauuugauuaacaaaaaaccguagaccuccuacgugagguauagcuagaaauugguucuaucgguugagagucuuuguacuauuagccauggaggacuauuugucuagcuuagaggccgcgagagagcucguccggacggagcuggagcccaagcguaaccucauagccagcuuagaguccgacgaucccgauccgguaauagcgccagcgguaaaaccaaaacaucccaagccaugccugagcacuaaagaagaggaucaucuccccucucuucgccuacuauucggcgcaaaacgagacaccucggugggcguagagcagacucuccacaagcgucucugcgcuugucucgacgguuaccugaccaugacgaagaaagaggccaaugccuuuaaggccgcggcugaagcagcagcauuagcagucauggacauuaagauggagcaucagcgccaggaucuagaggaucugaccgcugcuaucccuaggauagaauucaaacucaaugccauccuggaaaacaacaaggagauagccaaggcuguaacugcugcuaaggagauggagcgggagaugucguggggggaaagcgccgccagcucgcucaagucugucacccuagaugagucguuuaggggcccugaagagcuuucagagucauuuggcauccgauauaaggucagaaccuggaaugaguucaagaaggcgcuggaaaccagcauuguggaccugaggccuagcccuguuucauuuagggaauuacggacuauguggcugucucuugacaccuccuuuaggcucauuggguuugccuucauucccacaugcgagcgccuggagaccaaagccaaaugcaaggagacaaggacucuacucccccuugcagagucgaucaugcgaagaugggaccugcgggauccaaccaucuuggagaaagccugcguaguaaugaugauccgugggaaugagauugcaucgcugaaucagguaaaagauguucucccgaccacaauucguggguggaagaucgcuuauuagucacugcucccauuagucccacuagacggcauacuuccauuccgcccuuuaauuccccugucagacacucaugcuccgaaaucacuaaccauccuuguccaccaagcaauacgcauauucaguagcacugcaucucgcccucccccuaucaagccccagcgcugcagaucuucaccacauauauacaugcaucaacuacaugugauuuagaaaaaaccagacccuucacggguaauagccuaacucacgaacguuccucucguuucguaugauaaggccuuaagcauugucgauacggucguuaugcgucgguucuuuuuaggagagagcagugccccugcgagggacugggaguccgagcgaccuccccccuaugcuguugaggucccucaaagucacgggauaagagucaccggguacuuccagugcaacgagcguccgaaauccaagaagacccuccacagcuucgccguaaaacucugcgacgcaauuaagccgguucgagcggaugcucccagcuugaagauagcaauauggacggcucuagaucuggccuucgugaaaccucccaauggaacuguaacaauagaugcggcggugaaagcuacaccgcuaaucgggaacacccaguacaccguaggcgaugaaaucuuccagaugcuagggagaagggguggccugaucgucaucaggaacuuaccccaugauuauccucgaacguugauugaguucgccucucccgagccuugagcaccagggcaucgguccgcccgcccugugaucucccguagccgggcucagcgaucaagccggcccgggucgggggggacuggugcaacacaaggggcggcaguggacgcugauuaacaaaaaaccaccuauauagaccccucacggucuuagacucuguugccagcugacaaccaacacacaagacaucucucugauucagccgacccgaucgauuccuccccacccaauuccuaccaacgcacuccucacaagcuccaccggcgcgccauguugagacuuuuucucuuuuguuucuuggccuuaggagcccacuccaaauuuacuauaguauucccucaucaucaaaaagggaauuggaagaaugugccuuccacauaucauuauugcccuucuaguucugaccagaauuggcauaaugauuugacuggaguuagucuucaugugaaaauucccaaaagucacaaagcuauacaagcagauggcuggaugugccacgcugcuaaaugggugacuacuugugacuucagaugguacggacccaaauacaucacgcauuccauacacucuaugucacccacccuagaacagugcaagaccaguauugagcagacaaagcaaggaguuuggauuaauccaggcuuucccccucaaagcugcggauaugcuacagugacggaugcagaggugguuguuguacaagcaacaccucaucauguguugguugaugaguacacaggagaauggauugacucacaauugguggggggcaaauguuccaaggagguuugucaaacgguucacaacucgaccguguggcaugcugauuacaagauuacagggcugugcgagucaaaucuggcaucaguggauaucaccuucuucucugaggauggucaaaagacgucuuugggaaaaccgaacacuggauucaggaguaaucacuuugcuuacgaaaguggagagaaggcaugccguaugcaguacugcacacaaugggggauccgacuaccuucuggaguaugguuugaauuaguggacaaagaucucuuccaggcggcaaaauugccugaauguccuagaggauccaguaucucagcuccuucucagacuucuguggauguuaguuugauacaagacguagagaggaucuuagauuacucucuaugccaggagacguggaguaagauacgagccaagcuuccuguaucuccaguagaucugaguuaucucgccccaaaaaauccagggagcggaccggccuucacuaucauuaauggcacuuugaaauauuucgaaacaagauacaucagaguugacauaaguaaucccaucaucccucacauggugggaacaaugaguggaaccacgacugagcgugaauuguggaaugauugguauccauaugaagacguagagauugguccaaaugggguguugaaaacucccacugguuucaaguuuccgcuguacaugauugggcacggaauguuggauuccgaucuccacaaauccucccaggcucaagucuucgaacauccacacgcaaaggacgcugcaucacagcuuccugaugaugagacuuuauuuuuuggugacacaggacuaucaaaaaacccaguagaguuaguagaaggcugguucaguagcuggaagagcacauuggcaucguucuuucugauuauaggcuuggggguugcauuaaucuucaucauucgaauuauuguugcgauucgcuauaaauacaaggggaggaagacccaaaaaauuuacaaugaugucgagaugagucgauugggaaauaaauaaggcgcgccagaggacugcgauuguugaguggagccggcaaacccuaggccuauuccgauuuagaaaaaaccagaccucucacgaggucuuuucuacuagcuggguuuuccucauucuauccagagccgcggccgcauggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguaagcggccgccaaacccuaggccuauuccgauuuagaaaaaaccagaccucucacgaggucuuuucuacuagcuggguuuuccucauucuauccagagccauggccuucgacccgaacuggcagagagaagguuaugaaugggauccgucaagugagggcagaccgaccgaugagaacgaagacgacagaggucaucggccaaaaacgagacuucguacauuccuugcccgcacguuaaauagcccuauccgagcccuauucuacacaauauuccuaggaauucgagcgguuugggacggguucaaaagacuccuaccugugaggaccgaaaaggguuaugcgagguuuucugagugcgucacauauggaaugaucggaugugaugaguguguaauagacccggugaggguugucauugagcugaccgagaugcaguuaccgauuaaaggcaaaggcucuacgagguugagagcaaugauaacugaagaccuucucacggggaugcgcacagccgugccucagaucagagugagaucgaagauccuagcagagcgguuagggagagcaaucggccgagagaccuugccggcaaugauccaucaugagugggcauuugugauggggaagauucucacuuucauggcagacaauguggguaugaacgcugacacggucgagggcguucuaucacuaucagaggucacacggcgaugggauaucggcaacucuguguccgcaguguucaauccugauggccuuacuaucagaguagaaaacacggguuacaucaugaccagagagacugccugcaugaucggagacauucaugcucaauuugcaauccaauaccuagcugcauaccuagacgaggugaucggcacaaggacgucucucucacccgccgaacugaccucucucaaacuauggggacuuaacguccugaaacuccuaggacggaacgguuaugaggugaucgccugcauggagcccauaggguacgcuguccugaugaugggaagagacaggaguccugaucccuaugucaaugacaccuauuuaaacagcauccucucagaauucccugucgacucugacgcucgagccugcguugaagcccucuuaacuaucuauaugagcuucggcacaccccauaaagucucggacgcauucggccucuucagaauguugggacauccgaugguugauggagcugacgggauugaaaagaugcgaagguuaagcaagaaggucaagaucccagaccagucuacagcgaucgaccucggggcuaucauggccgaacuguuugugcggaguuucguaaagaagcacaaaagguggcccaacugcuccaucaaucucccgccacgacaccccuuccaccacgcccgccuauguggguaugucccggcugaaacccauccccuaaacaacacugcauccugggcggcuguggaguucaaccaggaauucgagccgccgagacaguacaaccuugcagacaucauugaugacaagucgugcucucccaacaagcaugagcuauauggugcuuggaugaagucaaaaacagcuggguggcaggaacaaaagaagcucauacuccgaugguucacugagaccaugguuaaaccuucggagcuccuggaagagauugaugcacacggcuuccgagaagaggauaaguugauuggauuaacaccaaaggagagagagcugaaauuaacaccaagaauguucuccuugaugacauucaaguucagaaccuaccaaguccucacugagaguauggucgccgaugagauccucccgcacuucccccagaucaccaugaccauguccaaccacgaacucacaaagagguugauuagcagaacgagaccucaaucuggaggagggcgugauguucacaucaccgugaacauagauuuccagaaauggaacacaaacaugagacacggacuggucaaacaugucuucgagcgacuggacaaccucuuuggcuucaccaacuuaaucagacgaacucaugaauacuuccaggaggcgaaauacuaucuggcugaagauggaacuaaucugucguucgacaggaacggggaguuaauagauggcccauacguuuacaccggaucauacggggggaacgagggguuacgacagaagcccuggacaauaguuaccguguguggaauauacaagguagcuagagaccugaaaaucaaacaucagaucaccggucagggagauaaucagguggucacccuaauauuuccggaucgagaguugccuucagauccgguggagaggagcaaguacuguagagacaagagcagucaguuccugacacgucucagucaauauuucgcugagguugguuugcccgucaagacugaagagacauggaugucaucacgucucuaugcuuacgguaagcgcauguucuuagagggaguuccacuuaagauguuucucaagaagauaggcagagcuuucgcccucucgaaugaguuugucccgucccucgaggaagaucuggccagagucuggagugccaccagcgcagcgguagagcuugaccuaacucccuacguaggauauguccucgggugcugcuugucugcgcaggcgaucagaaaucaccucaucuacuccccuguucuggagggcccucugcugguuaaggccuacgagcguaaguucauuaacuacgacggaggaacaaagcggggggcgaugcccggccuacguccaaccuuugagagccuagucaaaaguaucugcuggaagccaaaggccaucggaggguggccgguauugauguuagaagaucucaucaucaaaggguucccugauccggcgacuagcgcccuggcucaauugaagucaauggugccauauaccucugguaucgaccgggagaucauacuuuccugucucaaccuucccuuaucgucggugguaucuccgucaauguuguuaaaggacccggcggccaucaacaccaucacaaccccguccgcgggcgacauccugcaagaggucgccagagacuauguuaccgauuacccacuccaaaacccgcagcucagagcaguggucaagaacgugaagaccgagcuagacacauuggccagugacuuauucaaaugugaaccuuucuuuccuccuuuaaugagcgauaucuucucggcaucucucccggcauaucaagacaggauuguucgcaagugcuccacgacuucuacaaucaggagaaaagcugccgagaggggcuccgacucucuccucaaccggaugaaaaggaaugagaucaauaagaugauguuacaucuuugggcuaccuggggaaggagcccucuggccagauuagacaccagaugucucacaaccugcaccaagcaauuagcccaacaguaucggaaccagucuuggggaaagcagauccauggagucucagucggccaccccuuagaacuguucggucgaauaacacccagccauagaugccuacaugaggaggaccacggagauuuccugcaaaccuucgccagcgagcaugugaaccaaguggacaccgacaucaccacaacucuggggccguucuacccuuacauaggcucggagacgcgagaacgggcagucaagguucgaaaaggagugaauuacguaguugagccgcuucugaaacccgcaguucgacuacuaagagccauuaauugguucauucccgaggagucagaugcgucccauuugcugagcaaucuauuagcgucuguuaccgacaucaauccucaagaccacuacucaucuaccgaaguaggggggggcaacgccguccaucgcuacagcugccgacuauccgacaaauugagcagagucaacaacuuauaucaguugcauacuuauuuaucugucacaacagagcgguugaccaaguacagucgaggaucaaaaaacacugacgcacacuuccagagcaugaugauuuaugcacaaagccgucauauagaccucaucuuggagucucugcacaccggagagaugguaccguuggagugucaucaucacauugagugcaaucacuguauagaggauauacccgacgagccaaucacgggggacccggcuuggacugaagucaaguuuccuucaaguccucaggagcccuuucuuuacaucaggcaacaagaucugccggucaaagacaaacucgagccugugccucgcaugaacaucguccgucuugccggauuggguccggaggcgauuagugagcuagcgcacuacuuuguugcauuccgaguuauccgggcgucagagacggaugucgacccuaacgauguucucucguggaccuggcugagccgaauugauccugacaaauugguugaguauaucgugcauguguucgcuucacuggaauggcaucauguauuaaugucaggcgugagugugagcgucagagaugcauucuuuaagaugcuagugucuaaaagaaucucagagacuccgcuaaguucauucuauuaucuggccaaccuguucguugacccucagacucgcgaagcacuaaugagcucuaaauacggguucagcccccccgccgagacaguccccaacgcaaaugccgccgcagccgaaauaagaagaugcugugcgaacagugcgccgucgaucuuagaaucagcccuucacagccgugagguuguuuggaugccaggaacgaacaauuauggagacguugucaucuggucucauuacauuagauuacgguucagcgaaguuaaacuaguugacauuacacgauaucagcagugguggagacagucugagcgagaccccuacgauuuggucccggacaugcagguucuugagagcgaccuagauacgcugaugaaacggauaccgaggcucaugcgcaaggcgagacgucccccucuucagguaauucgagaggaccuggaugucgcagucaucaaugcugaucaucccgcucacucugugcuucagaacaaauacaggaaauugauuuucagagagccgaagauuaucacgggagcuguguacaaguaccucucccuaaaaucagaguugacagaguucaccucagcaauggugaucggagacggaacuggagguaucaccgccgccaugauggccgaugggauagaugugugguaucagacgcucgucaacuaugaccacgugacacaacagggauuauccguacaagccccggcagcauuggaucuucugcgcggggcacccucugguaggcucuugaauccgggaagauucgcaucauuugggucugaccuaacugacccucgauuuacagccuacuuugaucaauaucccccguucaagguggacacucuauggucugacgcagagggcgacuuuugggacaagccuuccaaguugaaucaauacuuugagaacaucauugcuuugagacaucgguucgugaagacaaauggacagcuugucgugaagguguaucugacucaagacacugcuaccacaauugaagcauucagaaagaagcuguccccaugcgccaucaucgugucucucuucucgacggaaggcuccacagaaugcuucguccuaagcaaucucaucgcaccagacaccccugucgaccuugagaugguggagaauaucccuaaacuaacaucccuuguuccccagaggacgacagugaaaugcuauucccgacgaguagcgugcaucaguaaaagguggggacuuuucagaucuccgagcauagcccuugaaguccaaccguuccuucacuacaucacaaaggucaucucagacaaaggaacacaacugagucucauggcgguagcugacacaaugaucaacaguuacaagaaggcuaucucaccccgaguguucgaucuacaccggcauagggccgcacuggguuucgggaggagauccuugcaucucaucugggggaugaucaucucaccaaucgcuuaccagcauuuugagaauccggccaaguugauggauguccuggacauguugaccaauaacaucucagcuuucuuaucgauaucgucgucaggauuugaccugucauuuagugucagugcagaccgagauguccggauugacagcaaacuugucagacucccgcuauucgaaggaucagaccuaaaauucaugaaaaccaucaugucuacccucggaucuguguucaaccaggucgagccuuuuaaggggaucgccauaaacccuucuaaacuaaugacugucaagaggacacaggaguuacguuacaacaaccuaauuuacacuaaggaugccauccuauuccccaaugaagcggcaaaaaacacugccccgcuucgagccaacaugguauaccccguccggggagaucuauucgccccuaccgaucgcauaccaaucaugacucuagucagcgaugagacaacaccucagcacucuccuccagaggaugaggcauaacugaauccucccugaaggcucacaugucccacgcgacgcaagauauaacgacaagcaacucgcccuauuaacugugauuaauaaaaaaccgauuauucaguugcuugagggaguuucaauccguucaguguaugauaggaaguuucugagaugguggggauuagggggcaccuagaguauguuuguucguuuuaugcgucgu

SEQ ID NO: 20 - protein, 412 aa, Farmington virus
MARPLAAAQHLITERHSLQATLSRASKTRAEEFVKDFYLQEQYSVPTIPTDDIAQSGPMLLQAILSEEYTKATDIAQSILWNTPTPNGLLREHLDADGGGSFTALPASAIRPSDEANAWAARISDSGLGPVFYAALAAYIIGWSGRGETSRVQQNIGQKWLMNLNAIFGTTITHPTTVRLPINVVNNSLAVRNGLAATLWLYYRSSPQSQDAFFYGLIRPCCSGYLGLLHRVQEIDEMEPDFLSDPRIIQVNEVYSALRALVQLGNDFKTADDEPMQVWACRGINNGYLTYLSETPAKKGAVVLMFAQCMLKGDSEAWNSYRTATWVMPYCDNVALGAMAGYIQARQNTRAYEVSAQTGLDVNMAAVKDFEASSKPKAAPISLIPRPADVASRTSERPSIPEVDSDEELGGM

SEQ ID NO: 21 - protein, 316 aa, Farmington virus
MEDYLSSLEAARELVRTELEPKRNLIASLESDDPDPVIAPAVKPKHPKPCLSTKEEDHLPSLRLLFGAKRDTSVGVEQTLHKRLCACLDGYLTMTKKEANAFKAAAEAAALAVMDIKMEHQRQDLEDLTAAIPRIEFKLNAILENNKEIAKAVTAAKEMEREMSWGESAASSLKSVTLDESFRGPEELSESFGIRYKVRTWNEFKKALETSIVDLRPSPVSFRELRTMWLSLDTSFRLIGFAFIPTCERLETKAKCKETRTLLPLAESIMRRWDLRDPTILEKACVVMMIRGNEIASLNQVKDVLPTTIRGWKIAY

SEQ ID NO: 22 - protein, 2129 aa, Farmington virus
MAFDPNWQREGYEWDPSSEGRPTDENEDDRGHRPKTRLRTFLARTLNSPIRALFYTIFLGIRAVWDGFKRLLPVRTEKGYARFSECVTYGMIGCDECVIDPVRVVIELTEMQLPIKGKGSTRLRAMITEDLLTGMRTAVPQIRVRSKILAERLGRAIGRETLPAMIHHEWAFVMGKILTFMADNVGMNADTVEGVLSLSEVTRRWDIGNSVSAVFNPDGLTIRVENTGYIMTRETACMIGDIHAQFAIQYLAAYLDEVIGTRTSLSPAELTSLKLWGLNVLKLLGRNGYEVIACMEPIGYAVLMMGRDRSPDPYVNDTYLNSILSEFPVDSDARACVEALLTIYMSFGTPHKVSDAFGLFRMLGHPMVDGADGIEKMRRLSKKVKIPDQSTAIDLGAIMAELFVRSFVKKHKRWPNCSINLPPRHPFHHARLCGYVPAETHPLNNTASWAAVEFNQEFEPPRQYNLADIIDDKSCSPNKHELYGAWMKSKTAGWQEQKKLILRWFTETMVKPSELLEEIDAHGFREEDKLIGLTPKERELKLTPRMFSLMTFKFRTYQVLTESMVADEILPHFPQITMTMSNHELTKRLISRTRPQSGGGRDVHITVNIDFQKWNTNMRHGLVKHVFERLDNLFGFTNLIRRTHEYFQEAKYYLAEDGTNLSFDRNGELIDGPYVYTGSYGGNEGLRQKPWTIVTVCGIYKVARDLKIKHQITGQGDNQVVTLIFPDRELPSDPVERSKYCRDKSSQFLTRLSQYFAEVGLPVKTEETWMSSRLYAYGKRMFLEGVPLKMFLKKIGRAFALSNEFVPSLEEDLARVWSATSAAVELDLTPYVGYVLGCCLSAQAIRNHLIYSPVLEGPLLVKAYERKFINYDGGTKRGAMPGLRPTFESLVKSICWKPKAIGGWPVLMLEDLIIKGFPDPATSALAQLKSMVPYTSGIDREIILSCLNLPLSSVVSPSMLLKDPAAINTITTPSAGDILQEVARDYVTDYPLQNPQLRAVVKNVKTELDTLASDLFKCEPFFPPLMSDIFSASLPAYQDRIVRKCSTTSTIRRKAAERGSDSLLNRMKRNEINKMMLHLWATWGRSPLARLDTRCLTTCTKQLAQQYRNQSWGKQIHGVSVGHPLELFGRITPSHRCLHEEDHGDFLQTFASEHVNQVDTDITTTLGPFYPYIGSETRERAVKVRKGVNYVVEPLLKPAVRLLRAINWFIPEESDASHLLSNLLASVTDINPQDHYSSTEVGGGNAVHRYSCRLSDKLSRVNNLYQLHTYLSVTTERLTKYSRGSKNTDAHFQSMMIYAQSRHIDLILESLHTGEMVPLECHHHIECNHCIEDIPDEPITGDPAWTEVKFPSSPQEPFLYIRQQDLPVKDKLEPVPRMNIVRLAGLGPEAISELAHYFVAFRVIRASETDVDPNDVLSWTWLSRIDPDKLVEYIVHVFASLEWHHVLMSGVSVSVRDAFFKMLVSKRISETPLSSFYYLANLFVDPQTREALMSSKYGFSPPAETVPNANAAAAEIRRCCANSAPSILESALHSREVVWMPGTNNYGDVVIWSHYIRLRFSEVKLVDITRYQQWWRQSERDPYDLVPDMQVLESDLDTLMKRIPRLMRKARRPPLQVIREDLDVAVINADHPAHSVLQNKYRKLIFREPKIITGAVYKYLSLKSELTEFTSAMVIGDGTGGITAAMMADGIDVWYQTLVNYDHVTQQGLSVQAPAALDLLRGAPSGRLLNPGRFASFGSDLTDPRFTAYFDQYPPFKVDTLWSDAEGDFWDKPSKLNQYFENIIALRHRFVKTNGQLVVKVYLTQDTATTIEAFRKKLSPCAIIVSLFSTEGSTECFVLSNLIAPDTPVDLEMVENIPKLTSLVPQRTTVKCYSRRVACISKRWGLFRSPSIALEVQPFLHYITKVISDKGTQLSLMAVADTMINSYKKAISPRVFDLHRHRAALGFGRRSLHLIWGMIISPIAYQHFENPAKLMDVLDMLTNNISAFLSISSSGFDLSFSVSADRDVRIDSKLVRLPLFEGSDLKFMKTIMSTLGSVFNQVEPFKGIAINPSKLMTVKRTQELRYNNLIYTKDAILFPNEAAKNTAPLRANMVYPVRGDLFAPTDRIPIMTLVSDETTPQHSPPEDEA

SEQ ID NO: 23 - protein, 148 aa, Farmington virus
MRRFFLGESSAPARDWESERPPPYAVEVPQSHGIRVTGYFQCNERPKSKKTLHSFAVKLCDAIKPVRADAPSLKIAIWTALDLAFVKPPNGTVTIDAAVKATPLIGNTQYTVGDEIFQMLGRRGGLIVIRNLPHDYPRTLIEFASPEP

SEQ ID NO: 24 - protein, 512 aa, Maraba virus
MLRLFLFCFLALGAHSKFTIVFPHHQKGNWKNVPSTYHYCPSSSDQNWHNDLTGVSLHVKIPKSHKAIQADGWMCHAAKWVTTCDFRWYGPKYITHSIHSMSPTLEQCKTSIEQTKQGVWINPGFPPQSCGYATVTDAEVVVVQATPHHVLVDEYTGEWIDSQLVGGKCSKEVCQTVHNSTVWHADYKITGLCESNLASVDITFFSEDGQKTSLGKPNTGFRSNHFAYESGEKACRMQYCTQWGIRLPSGVWFELVDKDLFQAAKLPECPRGSSISAPSQTSVDVSLIQDVERILDYSLCQETWSKIRAKLPVSPVDLSYLAPKNPGSGPAFTIINGTLKYFETRYIRVDISNPIIPHMVGTMSGTTTERELWNDWYPYEDVEIGPNGVLKTPTGFKFPLYMIGHGMLDSDLHKSSQAQVFEHPHAKDAASQLPDDETLFFGDTGLSKNPVELVEGWFSSWKSTLASFFLIIGLGVALIFIIRIIVAIRYKYKGRKTQKIYNDVEMSRLGNK

(19) United States
(12) Patent Application Publication
(10) Pub. No.: US 2015/0307559 A1
(43) Pub. Date: Oct. 29, 2015

(54) COMPOSITIONS AND METHODS FOR THE TREATMENT OF BRAIN CANCERS

(71) Applicants: CHILDREN'S HOSPITAL OF EASTERN ONTARIO RESEARCH INTITUTE INC., Ottawa (CA); OTTAWA HOSPITAL RESEARCH INSTITUTE, Ottawa (CA)

(72) Inventors: David STOJDL, Ottawa (CA); John Cameron BELL, Ottawa (CA)

(21) Appl. No.: 14/651,761

(22) PCT Filed: Dec. 12, 2012
(86) PCT No.: PCT/CA2012/050893

§ 371 Date: Jun. 12, 2015

(51) Int. Cl. C07K 14/005; A61K 35/766; C12N 7/00

(52) U.S. Cl. CPC C07K 14/005; C12N 7/00; A61K 35/766; C12N 2760/20233; C12N 2760/14033

(57) ABSTRACT

Described herein is an isolated viral particle having a genome that includes open reading frames that encode: Maraba proteins N, P, and L, or variants thereof; as well as Maraba protein M or protein delta 51M, or variants thereof; and a Bahia Grande G protein, a LCMV G protein, or an Ebola G protein. Maraba protein N may have a sequence which includes SEQ ID NO: 1. Maraba protein P may have a sequence which includes SEQ ID NO: 2. Maraba protein L may have a sequence which includes SEQ ID NO: 3. Maraba proteins M and delta 1M may have sequence which include SEQ ID NO: 4 and 5, respectively. Bahia Grande G protein may have a sequence which includes SEQ ID NO: 6. LCMV G protein may have a sequence which includes SEQ ID NO: 7. Ebola G protein may have a sequence which includes SEQ ID NO: 8.

### FIELD

The present disclosure relates to rhabdovirus chimeras and their use as an oncolytic treatment. More specifically, the present disclosure relates to Maraba rhabdovirus chimeras and its use in the treatment of primary and secondary brain cancers.

### BACKGROUND

Brain tumours are composed of cells that exhibit unrestrained growth in the brain. They can be benign (that is, noncancerous) or malignant (that is, cancerous). Cancerous brain tumours are further classified as either primary or secondary tumours.

Primary tumours start in the brain, whereas secondary tumours spread to the brain from another site such as breast or lung. Secondary tumours may also be referred to as metastatic. A secondary (that is, metastatic) brain tumour occurs when cancer cells spread to the brain from a primary cancer in another part of the body. Secondary tumours are three times more common than primary tumours of the brain. All metastatic brain tumours are malignant.

Brain tumours are generally named and classified according to the following: the type of brain cells from which they originate, or the location in which the cancer develops. The biological diversity of these tumours, makes classification difficult. About 80% of malignant primary brain tumours are known collectively as gliomas (that is, they originate in glial cells) and are classified into 4 grades reflecting the degree of malignancy.

Brain cancer is the leading cause of cancer-related death in patients younger than age 35 and accounts for roughly 10% of all cancers diagnosed in North America. Treatment of brain tumours is complicated by the fact that there are more than 120 different types, which range from low grade astrocytomas to grade 4 glioblastoma multiforme (GBM).

Malignant gliomas, such as GBM, are by far the most common brain cancer found in adults but are the fastest growing and most malignant of the primary brain tumours and therefore are the most difficult to treat. Even with aggressive single and multimodal treatment options such as surgery, chemotherapy, radiation and small molecule inhibitors, the survival has remained unchanged over the past three decades with a median survival of less than one year after diagnosis.

Reasons for the failure of conventional treatments is multifactorial including the highly infiltrative/invasive nature of GBM, limitation of drug delivery through the blood brain barrier and neural parenchyma, and genetic heterogeneity resulting in intrinsic resistance to available treatments and the rise of aggressive resistant clones. Therefore, there is a dire requirement for new treatment options, which has led to the renaissance of oncolytic viral therapy for brain cancers in general and GBM in particular.

Vesicular stomatitis virus (VSV) is a potent oncolytic rhabdovirus that infects and kills a broad range of tumour cell types (Brun et al., Mol Ther 18:1440-1449, 2010). As with other rhabdoviruses, neurotropism with subsequent neurovirulence, as well as a highly potent nAb response remain problems (Diallo et al., Methods Mol Biol 797:127-140, 2011). Although VSV is known to be effective by systemic delivery in neurological tumour models (Cary et al., J Virol 85:5708-5717, 2011; Lun et al., J Natl Cancer Inst 98: 1546-1547, 2006; Wollmann et al., J Virol 84:1563-1573, 2010), its inherent neurotoxicity has hindered its consideration as a clinical candidate (Hoffmann et al., J Gen Virol 91:2782-2793, 2010; Sur et al., Vet Pathol 40:512-520, 2003).

Maraba is a recently characterized oncolytic rhabdovirus that shares some sequence similarity, a similar yet more potent oncolytic spectrum, and similar neurotoxicity profile to VSV (Brun et al., Mol Ther 18:1440-1449, 2010). The rhabdoviruses VSV and Maraba constitute some of the most efficacious viruses being tested preclinically. However, a desired route of viral administration for brain cancer is intracerebral delivery, which is not currently possible with either VSV or Maraba due to their inherent neurotoxicity.

It is desirable to provide an oncolytic viral therapy for the treatment of cancers, and more specifically for the treatment of brain cancers, that obviates or mitigates at least one disadvantage of previous oncolytic viral therapies.

### SUMMARY

It is an object of the present disclosure to obviate or mitigate at least one disadvantage of previous oncolytic viral therapies. In some examples, the oncolytic viral therapy may exhibit reduced levels of neurotoxicity.

According to one aspect of the present disclosure, there is provided an isolated viral particle having a genome that includes open reading frames that encode: a protein having a sequence comprising SEQ ID NO: 1, or a variant thereof; a protein having a sequence comprising SEQ ID NO: 2, or a variant thereof; a protein having a sequence comprising SEQ ID NO: 3, or a variant thereof; a protein having a sequence comprising SEQ ID NO: 4 or 5, or a variant thereof; and a protein having a sequence comprising SEQ ID NO: 6, 7 or 8.

The variant of a reference protein may be a protein having a sequence which is at least 75%, at least 80%, at least 85%, at least 90%, or at least 95% identical to the sequence of the reference protein, and the variant protein maintains the same biological function as the reference protein.

The genome may include an open reading frame that encodes a protein having a sequence comprising SEQ ID NO: 6. Alternatively, the genome may include an open reading frame that encodes a protein having a sequence comprising SEQ ID NO: 7. Alternatively, the genome may include an open reading frame that encodes a protein having a sequence comprising SEQ ID NO: 8.

The viral genome may include open reading frames that encode: a protein having a sequence comprising SEQ ID NO: 1; a protein having a sequence comprising SEQ ID NO: 2; a protein having a sequence comprising SEQ ID NO: 3; a protein having a sequence comprising SEQ ID NO: 5; and a protein having a sequence comprising SEQ ID NO: 7.

According to another aspect of the present disclosure, there is provided an isolated viral particle that includes an RNA polynucleotide which has a sequence that includes: the reverse complement of the sequence defined by position 64 to position 1332 of SEQ ID NO: 10, or a conservative variant thereof; the reverse complement of the sequence defined by position 1393 to position 2190 of SEQ ID NO: 10, or a conservative variant thereof; the reverse complement of the sequence defined by position 4943 to position 11272 of SEQ ID NO: 10, or a conservative variant thereof; the reverse complement of the sequence defined by position 2256 to position 2945 of SEQ ID NO: 10, or a conservative variant thereof; the reverse complement of the sequence defined by position 3041 to position 4816 of SEQ ID NO: 10; and the reverse complements of promoters thereof.

The conservative variant of a sequence of nucleotides may be a sequence that is at least 75%, at least 80%, at least 85%, at least 90%, or at least 95% identical to the reference sequence of nucleotides. The conservative variant may be a sequence that includes one or more silent substitutions.

The isolated viral particle may be an isolated viral particle capable of producing a cDNA polynucleotide that includes a sequence according to SEQ ID NO: 9 when the virus is in a host cell.

The isolated viral particle may be an isolated viral particle that includes an RNA polynucleotide which includes a sequence according to SEQ ID NO: 10.

According to yet another aspect of the present disclosure, there is provided an isolated viral particle that includes an RNA polynucleotide which has a sequence that includes: the reverse complement of the sequence defined by position 64 to position 1332 of SEQ ID NO: 12, or a conservative variant thereof; the reverse complement of the sequence defined by position 1393 to position 2190 of SEQ ID NO: 12, or a conservative variant thereof; the reverse complement of the sequence defined by position 4664 to position 10993 of SEQ ID NO: 12, or a conservative variant thereof; the reverse complement of the sequence defined by position 2256 to position 2945 of SEQ ID NO: 12, or a conservative variant thereof; the reverse complement of the sequence defined by position 3041 to position 4537 of SEQ ID NO: 12; and the reverse complements of promoters thereof.

The conservative variant of a sequence of nucleotides may be a sequence that is at least 75%, at least 80%, at least 85%, at least 90%, or at least 95% identical to the reference sequence of nucleotides. The conservative variant may be a sequence comprising one or more silent substitutions.

The isolated viral particle may be an isolated viral particle capable of producing a cDNA polynucleotide that includes a sequence according to SEQ ID NO: 11 when the virus is in a host cell.

The isolated viral particle may be an isolated viral particle that includes an RNA polynucleotide which includes a sequence according to SEQ ID NO: 12.

According to still another aspect of the present disclosure, there is provide an isolated viral particle that includes an RNA polynucleotide which has a sequence that includes: the reverse complement of the sequence defined by position 64 to position 1332 of SEQ ID NO: 14, or a conservative variant thereof; the reverse complement of the sequence defined by position 1393 to position 2190 of SEQ ID NO: 14, or a conservative variant thereof; the reverse complement of the sequence defined by position 5195 to position 11524 of SEQ ID NO: 14, or a conservative variant thereof; the reverse complement of the sequence defined by position 2256 to position 2942 of SEQ ID NO: 14, or a conservative variant thereof; the reverse complement of the sequence defined by position 3038 to position 5068 of SEQ ID NO: 14; and the reverse complements of promoters thereof.

The conservative variant of a sequence of nucleotides may be a sequence that is at least 75%, at least 80%, at least 85%, at least 90%, or at least 95% identical to the reference sequence of nucleotides. The conservative variant may be a sequence comprising one or more silent substitutions.

The isolated viral particle may be an isolated viral particle capable of producing a cDNA polynucleotide that includes a sequence according to SEQ ID NO: 13 when the virus is in a host cell.

The isolated viral particle may be an isolated viral particle that includes an RNA polynucleotide which includes a sequence according to SEQ ID NO: 14.

According to an additional aspect of the present disclosure, there is provided a use of an isolated viral particle according to the present disclosure for the treatment of cancer. The cancer may be a brain cancer. The brain cancer may be a glioblastoma.

The isolated viral particle may be used to infect a cell where the infected cell is used for the treatment of cancer.

According to further aspect of the present disclosure, there is provided a use of an isolated viral particle according to the present disclosure for inducing a cytotoxic response in a person administered the virus. The cytotoxic response may be an anti-cancer response.

The isolated viral particle may be formulated for direct delivery to the central nervous system, outside the blood/brain barrier, inside the blood/brain barrier, or any combination thereof. The isolated viral particle may be formulated for administration via intrathecal injection, intravenous injection, intracranial injection, or any sequential or simultaneous combination thereof.

The isolated viral particle may be used to infect a cell where the infected cell is used to generate the cytotoxic response. The infected cell maybe formulated for direct delivery to the central nervous system, outside the blood/brain barrier, inside the blood/brain barrier, or any combination thereof. The infected cell may be formulated for administration via intrathecal injection, intravenous injection, intracranial injection, or any sequential or simultaneous combination thereof.

According to yet another aspect of the present disclosure, there is provided a method for treating cancer. The method includes administering an isolated viral particle according to the present disclosure to a patient having cancer. The cancer may be a brain cancer. The brain cancer may be a glioblastoma.

The isolated viral particle may be administered to the patient directly. The isolated viral particle may be administered directly to the central nervous system, outside the blood/brain barrier, inside the blood/brain barrier, or any combination thereof. The isolated viral particle may be administered to the patient via intrathecal injection, intravenous injection, intracranial injection, or any combination thereof sequentially or simultaneously.

The method may include infecting a cell with the isolated viral particle and administering the infected cell to the patient. The infected cell may be administered directly to the central nervous system, outside the blood/brain barrier, inside the blood/brain barrier, or any combination thereof. The infected cell may be administered to the patient intrathecally, intravenously, via intracranial injection, or any combination thereof sequentially or simultaneously.

According to still another aspect of the present disclosure, there is provided a method for inducing a cytotoxic response in a patient. The method includes administering an isolated viral particle according to the present disclosure to the patient.

The isolated viral particle may be administered to the patient directly. The isolated viral particle may be administered directly to the central nervous system, outside the blood/brain barrier, inside the blood/brain barrier, or any combination thereof. The isolated viral particle may be administered to the patient intrathecally, intravenously, via intracranial injection, or any combination thereof sequentially or simultaneously.

The method may include infecting a cell with the isolated viral particle and administering the infected cell to the patient. The infected cell may be administered directly to the central nervous system, outside the blood/brain barrier, inside the blood/brain barrier, or any combination thereof. The infected cell may be administered to the patient intrathecally, intravenously, via intracranial injection, or any combination thereof sequentially or simultaneously.

According to still another aspect of the present disclosure, there is provided a kit for the treatment of cancer in a patient. The kit includes: the isolated viral particle according to the present disclosure; and instructions for administration of the isolated viral particle to the patient.

The cancer maybe a brain cancer. The brain cancer may be a glioblastoma.

The isolated viral particle may be formulated for direct delivery to the central nervous system, outside the blood/brain barrier, inside the blood/brain barrier, or any combination thereof. The isolated viral particle may be formulated for administration via intrathecal injection, intravenous injection, intracranial injection, or any sequential or simultaneous combination thereof.

The isolated viral particle may be formulated for infection of a cell and the cell maybe formulated for delivery to the central nervous system, outside the blood/brain barrier, inside the blood/brain barrier, or any combination thereof. The cell may be for administration via intrathecal injection, intravenous injection, intracranial injection, or any sequential or simultaneous combination thereof.

Other aspects and features of the present disclosure will become apparent to those ordinarily skilled in the art upon review of the following description of specific examples in conjunction with the accompanying figures.

### BRIEF DESCRIPTION OF THE DRAWINGS

Embodiments of the present disclosure will now be described, by way of example only, with reference to the attached Figures.

FIG. 1 is a graph illustrating the identification of non-neurotoxic rhabdoviruses based on survival of Balb/C mice after a single intracerebral dose of the indicated virus (1e7 pfu). Animals were monitored for weight loss, piloerection, hind leg paralysis, morbidity and mortality.

FIG. 2A is a schematic illustration of G swapping MRB G with BG G or EB G.

FIGS. 2B and 2C are graphs illustration results from viability assays demonstrating viral attenuation in normal human astrocytes (NHA) and GM38 skin fibroblasts. Error bars represent standard error of the mean (SEM) of 4 biological replicates.

FIGS. 2D through 2K are graphs illustrating results from viability assays demonstrating MRBGG are cytolytic on human brain cancer cell lines. Viability was assayed using Alamar blue 72 h post treatment. Error bars represent SEM of 4 biological replicates.

FIG. 3A is a summary of the intracerebral toxicity of wild type FMT, BG, MS, MRB, and several engineered rhabdovirus strains in VSV and MRB vector backbones. MRBGG and Maraba EbG Δ51 are viruses according to the present disclosure.

FIG. 3B is a summary of the viral load in brain homogenates of animals sacrificed 3 months post intracerebral inoculation. Limit of detection is 10¹.

FIG. 3C shows pathology photos. Photos of FMT and MRBGG pathology of acutely infected Balb/C mice are indistinguishable from saline injected animals. Balb/C mice were inoculated intracerebrally with the indicated viruses (1e7 pfu) and sacrificed 48 hours post inoculation.

FIG. 3D is a graph illustrating the motor function of mice treated with non-neurotoxic rhabdoviruses and control mice. Motor function is not compromised after intracerebral injection of non-neurotoxic rhabdoviruses. Motor function was assessed by rotorod analysis measuring the latency to fall off an accelerating rod.

FIG. 3E is a graph illustrating the toxicity profile after a single IV injection of either FMT or MRBGG chimera at varying doses. Maximum tolerated dose (MTD) is equal to the highest dose not resulting in durable morbidity as measured by behaviour and weight.

FIG. 4A is an IVIS image of U87MG tumours post MRBGG or EbG IV treatment (3 doses 1e9 pfu) vs. control treatment with PBS. Systemic delivery of these viruses enhances efficacy in a human U87MG xenograft model.

FIG. 4B is a graph illustrating the flux plot, demonstrating a significant tumour regression in response to three IV doses (1e9 pfu) of MRBGG or EbG. Error bars represent SEM.

FIG. 4C is a Kaplan Meir survival plot of MRBGG (Log rank test P=0.01) and EbG (Log rank test P=0.01) IV treated animals.

FIG. 5 is a graph illustrating the oncolytic activity of a variety of viruses on a panel of human glioblastoma cells.

FIG. 6A is a graph illustrating the in vivo neurotoxicity of Maraba chimeric viruses according to the present disclosure vs. control viruses. The graph shows Kaplan Meir survival plots of Balb/C mice after a single intracerebral dose of the indicated virus (1e6 pfu).

FIG. 6B is a graph showing the weight variation of the animals of FIG. 6A.

FIG. 7A is a graph illustrating in vivo efficacy of maraba chimeras according to the present disclosure versus control viruses. The graph shows Kaplan Meir survival plots of CD-1 nude mice with U87MG tumors post treatment.

FIG. 7B is a graph showing the weight variation of the animals of FIG. 7A.

FIG. 8A is an IVIS image of U87MG tumours illustrating in vivo efficacy of PBS control in a human U87MG xenograft model. The image shows tumours pre and post (1 week, 2 weeks, 3 weeks, 4 weeks) treatment.

FIG. 8B is a flux plot illustrating a significant increase in tumour burden over time in untreated control animals.

FIG. 9A is an IVIS image of U87MG tumours illustrating in vivo efficacy of BG wild type (BG-WT) virus treatment in a human U87MG xenograft model. The image shows U87MG tumours post BG (1 week, 2 weeks, 3 weeks, 4 weeks) treatment (1 dose 1e7 pfu: IC).

FIG. 9B is a flux plot illustrating an initial moderate tumour regression in response to IC dose (1e7 pfu) of BG followed by a recurrence in tumour burden.

FIG. 10A is an IVIS image of U87MG tumours illustrating in vivo efficacy of FMT wild type (FMT-WT) virus treatment in a human U87MG xenograft model. The image shows U87MG tumours post FMT-WT (1 week, 2 weeks, 3 weeks, 4 weeks) treatment (1 dose 1e7 pfu: IC).

FIG. 10B is a flux plot demonstrating a significant tumour regression in response to IC dose (1e7 pfu) of FMT-WT.

FIG. 11A is an IVIS image of U87MG tumours illustrating in vivo efficacy of MRB BG(G) treatment in a human U87MG xenograft model. The image shows U87MG tumours post MRB BG(G) (1 week, 2 weeks, 3 weeks, 4 weeks) treatment (1 dose 1e7 pfu: IC).

FIG. 11B is a flux plot illustrating moderate tumour regression in response to IC dose (1e7 pfu) of MRB BGG.

FIG. 12A is an IVIS image of U87MG tumours illustrating in vivo efficacy of MRB FMT(G) treatment in a human U87MG xenograft model. The image shows U87MG tumours post MRB FMT(G) (1 weeks, 2 weeks, 3 weeks) treatment (1 dose 1 e7 pfu).

FIG. 12B is a flux plot demonstrating a significant tumour regression in response to IC dose (1e7 pfu) of MRB FMT G. However, all animals succumbed to neurotoxic effects of MRB FMT(G) treatment prior to 4 weeks post treatment.

FIG. 13A is an IVIS image of U87MG tumours illustrating in vivo efficacy of FMT MRB(G) treatment in a human U87MG xenograft model. The image shows U87MG tumours post FMT MRB(G) (1 week, 2 weeks, 3 weeks) treatment (1 dose 1e7 pfu: IC).

FIG. 13B is a flux plot illustrating a significant tumour regression in response to IC dose (1e7 pfu) of FMT MRB(G). However, all animals succumbed to neurotoxic effects of FMT MRB G treatment prior to 4 weeks post treatment.

FIG. 14A is an IVIS image of U87MG tumours illustrating in vivo efficacy of VSV-LCMV(G) treatment in a human U87MG xenograft model. The image shows U87MG tumours post VSV LCMV G (1 week, 2 weeks, 3 weeks, 4 weeks) treatment (1 dose 1e7 pfu: IC).

FIG. 14B is a flux plot illustrating a significant tumour regression in response to IC dose (1e7 pfu) of VSV-LCMV(G).

FIG. 15A is an IVIS image of U87MG tumours illustrating in vivo efficacy of MRB LCMV(G) treatment in a human U87MG xenograft model. The image shows U87MG tumours post MRB LCMV G (1 week, 2 weeks, 3 weeks, 4 weeks) treatment (1 dose 1e7 pfu: IC).

FIG. 15B is a flux plot illustrating a significant tumour regression in response to IC dose (1e7 pfu) of MRB-LCMV(G).

FIG. 16 is a graph illustrating the neutralizing antibody titres in Balb/C mice treated with wild type Maraba virus, attenuated VSV (VSV-Δ51), Maraba LCMV(G) chimera or VSV-LCMV(G) chimera.

### DESCRIPTION

### Definitions

Throughout the present disclosure, several terms are employed that are defined in the following paragraphs.

As used herein, the words “desire” or “desirable” refer to embodiments of the technology that afford certain benefits, under certain circumstances. However, other embodiments may also be desirable, under the same or other circumstances. Furthermore, the recitation of one or more desired embodiments does not imply that other embodiments are not useful, and is not intended to exclude other embodiments from the scope of the technology.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

“A” and “an” as used herein indicate “at least one” of the item is present; a plurality of such items may be present, when possible.

“About” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters.

As used herein, the term “and/or” includes any and all combinations of one or more of the associated listed items.

As used herein, a virus that has “reduced levels of neurotoxicity” or “reduced neurotoxicity” would be understood to refer to a virus that, when injected into the right striatum of a mouse brain at a given dose, results in a mouse with fewer signs of neurotoxicity (for example, weight loss, piloerection, hind leg paralysis, morbidity and mortality) than a mouse which is injected with the corresponding wild-type virus.

As used herein, a virus having “substantially no level of neurotoxicity” or “substantially no neurotoxicity” would be understood to refer to a virus that, when injected into a patient at an efficacious dose, results in no detectable signs of reduced motor function compared to the patient before injection with the virus using a standard protocol for that a patient of that species. For example, a virus having “substantially no neurotoxicity” would be understood to refer to a virus that, when injected into a mouse at 1e7 pfu results in a mouse with no detectable signs of reduced motor function as measured by time on a rotorod, compared to the mouse before injection with the virus.

### DETAILED DESCRIPTION

Of the more than 250 currently identified rhabdoviruses, the authors of the present disclosure tested several wild type rhabdoviruses and determined many to be effective at killing CNS tumour cell lines. Several of these potent viral isolates were also determined to demonstrate remarkable attenuation, resulting in 100% survival after intracerebral inoculation. This is in striking contrast to previously tested Maraba and VSV viruses. The authors of the present disclosure subsequently sequenced and engineered chimeric viruses to test alongside known non-neurotoxic wild type isolates.

Generally, the present disclosure provides systems, methods, uses, processes, articles, and compositions that relate to engineered chimeric Maraba rhabdoviruses, and related nucleotide and protein sequences thereof. For example, the present disclosure provides the use of chimeric Maraba rhabdovirus in oncolytic treatments, for example treatment of primary or secondary brain cancers.

Contemplated oncolytic viruses may be used to treat cancer by directly administering the virus to a patient, or by infecting a cell with the virus and administering the infected cell to the patient to deliver the virus. The cell to be infected by the virus may be a cancer cell from the patient, a normal immune cell, or a stem cell. In some examples, the cancer to be treated is brain cancer, such as malignant glioma. One example of a malignant glioma is glioblastoma.

Viral particles according to the present disclosure may contain no wild type plasmid, may contain no sequences which encode a wild-type Maraba G-protein, or both.

In one example of viral particles according to the present disclosure, there is provided an isolated viral particle having a genome that includes open reading frames that encode: Maraba proteins N, P, and L, or any variants thereof; as well as Maraba protein M or protein Δ51M, or any variants thereof; and a Bahia Grande G protein, a LCMV G protein, or an Ebola G protein.

Maraba protein N may have a sequence which includes SEQ ID NO: 1. Maraba protein P may have a sequence which includes SEQ ID NO: 2. Maraba protein L may have a sequence which includes SEQ ID NO: 3. Maraba proteins M and Δ051M may have sequence which include SEQ ID NO: 4 and 5, respectively. Bahia Grande G protein may have a sequence which includes SEQ ID NO: 6. LCMV G protein may have a sequence which includes SEQ ID NO: 7. Ebola G protein may have a sequence which includes SEQ ID NO: 8.

A variant of a reference protein may be a protein having a sequence which is at least 75%, at least 80%, at least 85%, at least 90%, or at least 95% identical to the sequence of the reference protein, and the variant protein maintains the same biological function as the reference protein. For example, a variant protein would be considered to maintain the same biological function as the reference protein if a viral particle which has been modified with the variant protein had the same cytotoxicity and neurotoxicity as a viral particle with the reference protein.

In a particular example, the isolated viral particle has a genome which includes open reading frames that encode proteins having sequences that include SEQ ID NOs: 1, 2, 3, 4, and 6.

In another example, the isolated viral particle has a genome which includes open reading frames that encode proteins having sequences that include SEQ ID NOs: 1, 2, 3, 4, and 7.

In still another example, the isolated viral particle has a genome which includes open reading frames that encode proteins having sequences that include SEQ ID NOs: 1, 2, 3, 4, and 8.

In a further example, the isolated viral particle has a genome which includes open reading frames that encode proteins having sequences that include SEQ ID NOs: 1, 2, 3, 5, and 6.

In still yet another example, the isolated viral particle has a genome which includes open reading frames that encode proteins having sequences that include SEQ

ID NOs: 1, 2, 3, 5, and 7.

In still a further example, the isolated viral particle has a genome which includes open reading frames that encode proteins having sequences that include SEQ ID NOs: 1, 2, 3, 5, and 8.

In another example of viral particles according to the present disclosure, there is provided an isolated viral particle comprising an RNA polynucleotide which has a sequence that includes: the reverse complement of the sequence defined by position 64 to position 1332 of SEQ ID NO: 10, or a conservative variant thereof; the reverse complement of the sequence defined by position 1393 to position 2190 of SEQ ID NO: 10, or a conservative variant thereof; the reverse complement of the sequence defined by position 4943 to position 11272 of SEQ ID NO: 10, or a conservative variant thereof; the reverse complement of the sequence defined by position 2256 to position 2945 of SEQ ID NO: 10, or a conservative variant thereof; the reverse complement of the sequence defined by position 3041 to position 4816 of SEQ ID NO: 10; and the reverse complements of promoters thereof.

A conservative variant may be a sequence that is at least 75%, at least 80%, at least 85%, at least 90%, or at least 95% identical to the reference sequence of nucleotides. A conservative variant may be a sequence comprising one or more silent substitutions.

A particular example of a viral particle according to the present disclosure is an isolated viral particle capable of producing a cDNA polynucleotide comprising a sequence according to SEQ ID NO: 9 when the virus is in a host cell.

A particular example of a viral particle according to the present disclosure is an isolated viral particle comprising an RNA polynuclotide comprising a sequence according to SEQ ID NO: 10.

In another example of viral particles according to the present disclosure, there is provided an isolated viral particle comprising an RNA polynucleotide which has a sequence that includes: the reverse complement of the sequence defined by position 64 to position 1332 of SEQ ID NO: 12, or a conservative variant thereof; the reverse complement of the sequence defined by position 1393 to position 2190 of SEQ ID NO: 12, or a conservative variant thereof; the reverse complement of the sequence defined by position 4664 to position 10993 of SEQ ID NO: 12, or a conservative variant thereof; the reverse complement of the sequence defined by position 2256 to position 2945 of SEQ ID NO: 12, or a conservative variant thereof; the reverse complement of the sequence defined by position 3041 to position 4537 of SEQ ID NO: 12; and the reverse complements of promoters thereof.

A conservative variant may be a sequence that is at least 75%, at least 80%, at least 85%, at least 90%, or at least 95% identical to the reference sequence of nucleotides. A conservative variant may be a sequence comprising one or more silent substitutions.

A particular example of a viral particle according to the present disclosure is an isolated viral particle capable of producing a cDNA polynucleotide comprising a sequence according to SEQ ID NO: 11 when the virus is in a host cell.

A particular example of a viral particle according to the present disclosure is an isolated viral particle comprising an RNA polynuclotide comprising a sequence according to SEQ ID NO: 12.

In another example of viral particles according to the present disclosure, there is provided an isolated viral particle comprising an RNA polynucleotide which has a sequence that includes: the reverse complement of the sequence defined by position 64 to position 1332 of SEQ ID NO: 14, or a conservative variant thereof; the reverse complement of the sequence defined by position 1393 to position 2190 of SEQ ID NO: 14, or a conservative variant thereof; the reverse complement of the sequence defined by position 5195 to position 11524 of SEQ ID NO: 14, or a conservative variant thereof; the reverse complement of the sequence defined by position 2256 to position 2942 of SEQ ID NO: 14, or a conservative variant thereof; the reverse complement of the sequence defined by position 3038 to position 5068 of SEQ ID NO: 14; and the reverse complements of promoters thereof.

A conservative variant may be a sequence that is at least 75%, at least 80%, at least 85%, at least 90%, or at least 95% identical to the reference sequence of nucleotides. A conservative variant may be a sequence comprising one or more silent substitutions.

A particular example of a viral particle according to the present disclosure is an isolated viral particle capable of producing a cDNA polynucleotide comprising a sequence according to SEQ ID NO: 13 when the virus is in a host cell.

A particular example of a viral particle according to the present disclosure is an isolated viral particle comprising an RNA polynuclotide comprising a sequence according to SEQ ID NO: 14.

According to another aspect of the present disclosure, an isolated viral particle according to the present disclosure may be used for the treatment of cancer. The cancer may be a brain cancer, for example a glioblastoma.

The isolated viral particle maybe used to infect a cell and the infected cell may be used for the treatment of cancer.

According to another aspect of the present disclosure, an isolated viral particle according to the present disclosure may be used to induce a cytotoxic response in a person administered the virus. The cytotoxic response may be an anti-cancer response. The isolated viral particle may be used to infect a cell and the infected cell maybe used to generate the cytotoxic response.

The isolated viral particle may be formulated for direct delivery to the central nervous system, outside the blood/brain barrier, inside the blood/brain barrier, or any combination thereof. The isolated viral particle may be formulated for administration via intrathecal injection, intravenous injection, intracranial injection, or any sequential or simultaneous combination thereof.

The infected cell may be formulated for direct delivery to the central nervous system, outside the blood/brain barrier, inside the blood/brain barrier, or any combination thereof. The infected cell may be formulated for administration via intrathecal injection, intravenous injection, intracranial injection, or any sequential or simultaneous combination thereof.

According to another aspect of the present disclosure, there is provided a method for treating cancer which includes administering an isolated viral particle according to the present disclosure to a patient having cancer. The cancer may be a brain cancer, for example a glioblastoma.

The isolated viral particle may be administered to the patient directly. The isolated viral particle may be administered directly to the central nervous system, outside the blood/brain barrier, inside the blood/brain barrier, or any combination thereof. The isolated viral particle may be administered to the patient intrathecally, intravenously, via intracranial injection, or any combination thereof sequentially or simultaneously.

The method may include infecting a cell with the isolated viral particle and administering the infected cell to the patient. The infected cell may be administered directly to the central nervous system, outside the blood/brain barrier, inside the blood/brain barrier, or any combination thereof. The infected cell may be administered to the patient intrathecally, intravenously, via intracranial injection, or any combination thereof sequentially or simultaneously.

According to another aspect of the present disclosure, there is provided a method for inducing a cytotoxic response in a patient which includes administering an isolated viral particle according to the present disclosure to the patient.

The isolated viral particle may be administered to the patient directly. The isolated viral particle may be administered directly to the central nervous system, outside the blood/brain barrier, inside the blood/brain barrier, or any combination thereof. The isolated viral particle may be administered to the patient intrathecally, intravenously, via intracranial injection, or any combination thereof sequentially or simultaneously.

The method may include infecting a cell with the isolated viral particle and administering the infected cell to the patient. The infected cell may be administered directly to the central nervous system, outside the blood/brain barrier, inside the blood/brain barrier, or any combination thereof. The infected cell may be administered to the patient intrathecally, intravenously, via intracranial injection, or any combination thereof sequentially or simultaneously.

According to another aspect of the present disclosure, there is provided a kit for the treatment of cancer in a patient. The kit includes an isolated viral particle according to the present disclosure and instructions for administration of the isolated viral particle to the patient.

The cancer may be a brain cancer, for example a glioblastoma.

The isolated viral particle may be formulated for direct delivery to the central nervous system, outside the blood/brain barrier, inside the blood/brain barrier, or any combination thereof. The isolated viral particle maybe formulated for administration via intrathecal injection, intravenous injection, intracranial injection, or any sequential or simultaneous combination thereof.

The isolated viral particle may be formulated for infection of a cell and the cell is for delivery to the central nervous system, outside the blood/brain barrier, inside the blood/brain barrier, or any combination thereof. The cell may be formulated for administration via intrathecal injection, intravenous injection, intracranial injection, or any sequential or simultaneous combination thereof.

In any of the above aspects, administration via one route may be combined with one or more other routes of administration. Administration of the viral particle via the different routes may be sequential and/or simultaneous. The route or mode of administration of a virus according to the present disclosure is not expected to affect the ability of the virus to infect and kill cancerous cells, regardless of whether the virus is administered directly or by first infecting a cell and administering the infected cell to the patient. Viruses according to the present disclosure, when administered either inside or outside the blood/brain, are expected to be able to cross the blood/brain barrier and infect cancerous cells on the other side of the blood/brain barrier.

Techniques for infecting a cell with a virus and using the infected cell to deliver the virus are discussed in, for example: Power A T, et al. Carrier cell-based delivery of an oncolytic virus circumvents antiviral immunity. Mol Ther. 2007 January; 15(1):123-30; and Tyler M A, et al. Neural stem cells target intracranial glioma to deliver an oncolytic adenovirus in vivo. Gene Ther. 2009 February; 16(2):262-78.

Polynucleotide and Amino Acid Sequences

Polynucleotides comprising nucleic acid sequences (e.g., DNA and RNA) and amino acid (e.g., protein) sequences are provided that may be used in a variety of methods and techniques known to those skilled in the art of molecular biology. These include isolated, purified, and recombinant forms of the listed sequences and further include complete or partial forms of the listed sequences. Non-limiting uses for amino acid sequences include making antibodies to proteins or peptides comprising the disclosed amino acid sequences. Non-limiting uses for the polynucleotide sequences include making hybridization probes, as primers for use in the polymerase chain reaction (PCR), for chromosome and gene mapping, and the like. Complete or partial amino acid or polynucleotide sequences can be used in such methods and techniques.

The present disclosure features the identification of polynucleotide sequences, including gene sequences and coding nucleic acid sequences, and amino acid sequences. In addition to the sequences expressly provided in the accompanying sequence listing, also included are polynucleotide sequences that are related structurally and/or functionally. Also included are polynucleotide sequences that hybridize under stringent conditions to any of the polynucleotide sequences in the sequence listing, or a subsequence thereof (e.g., a subsequence comprising at least 100 contiguous nucleotides). Polynucleotide sequences also include sequences and/or subsequences configured for RNA production and/or translation, e.g., mRNA, antisense RNA, sense RNA, RNA silencing and interference configurations, etc.

Polynucleotide sequences that are substantially identical to those provided in the sequence listing can be used in the compositions and methods disclosed herein. Substantially identical or substantially similar polynucleotide sequences are defined as polynucleotide sequences that are identical, on a nucleotide by nucleotide basis, with at least a subsequence of a reference polynucleotide. Such polynucleotides can include, e.g., insertions, deletions, and substitutions relative to any of those listed in the sequence listing. For example, such polynucleotides are typically at least about 70% identical to a reference polynucleotide selected from those in the sequence listing, or a subsequence thereof. For example, at least 7 out of 10 nucleotides within a window of comparison are identical to the reference sequence selected. Furthermore, such sequences can be at least about 70%, at least about 75%, at least about 80%, at least about 85%, at least about 90%, at least about 95%, at least about 98%, at least about 99%, or at least about 99.5%, identical to the reference sequence. Subsequences of these polynucleotides can include at least about 5, at least about 10, at least about 15, at least about 20, at least about 25, at least about 50, at least about 75, at least about 100, at least about 500, about 1000 or more, contiguous nucleotides or complementary subsequences. Such subsequences can be, e.g., oligonucleotides, such as synthetic oligonucleotides, isolated oligonucleotides, or full-length genes or cDNAs. Polynucleotide sequences complementary to any of the described sequences are included.

Amino acid sequences include the amino acid sequences represented in the sequence listing, and subsequences thereof. Also included are amino acid sequences that are highly related structurally and/or functionally. For example, in addition to the amino acid sequences in the sequence listing, amino acid sequences that are substantially identical can be used in the disclosed compositions and methods. Substantially identical or substantially similar amino acid sequences are defined as amino acid sequences that are identical, on an amino acid by amino acid basis, with at least a subsequence of a reference amino acid sequence. Such amino acid sequences can include, e.g., insertions, deletions, and substitutions relative to any of the amino acid sequences in the sequence listing. For example, such amino acids are typically at least about 70% identical to a reference amino acid sequence, or a subsequence thereof. For example, at least 7 out of 10 amino acids within a window of comparison are identical to the reference amino acid sequence selected. Frequently, such amino acid sequences are at least about 70%, at least about 75%, at least about 80%, at least about 85%, at least about 90%, at least about 95%, at least about 98%, at least about 99%, or at least about 99.5%, identical to the reference sequence. Subsequences of the amino acid sequences can include at least about 5, at least about 10, at least about 15, at least about 20, at least about 25, at least about 50, at least about 75, at least about 100, at least about 500, about 1000 or more, contiguous amino acids. Conservative variants of amino acid sequences or subsequences are also possible. Amino acid sequences can be cytotoxic, enzymatically active, enzymatically inactive, and the like.

Where the polynucleotide sequences are translated to form a polypeptide or subsequence of a polypeptide, nucleotide changes can result in either conservative or non-conservative amino acid substitutions. Conservative amino acid substitutions refer to the interchangeability of residues having functionally similar side chains. Conservative substitution tables providing functionally similar amino acids are well-known in the art. Table 1 sets forth examples of six groups containing amino acids that are “conservative substitutions” for one another. Other conservative substitution charts are available in the art, and can be used in a similar manner.

TABLE 1
Conservative Substitution Group

| | | | | |
|---|---|---|---|---|
| 1 | Alanine (A) | Serine (S) | Threonine (T) | |
| 2 | Aspartic acid (D) | Glutamic acid (E) | | |
| 3 | Asparagine (N) | Glutamine (Q) | | |
| 4 | Arginine (R) | Lysine (K) | | |
| 5 | Isoleucine (I) | Leucine (L) | Methionine (M) | Valine (V) |
| 6 | Phenylalanine (F) | Tyrosine (Y) | Tryptophan (W) | |

One of skill in the art will appreciate that many conservative substitutions yield functionally identical constructs. For example, as discussed above, owing to the degeneracy of the genetic code, “silent substitutions” (i.e., substitutions in a polynucleotide sequence which do not result in an alteration in an encoded polypeptide) are an implied feature of every polynucleotide sequence which encodes an amino acid. Similarly, “conservative amino acid substitutions,” in one or a few amino acids in an amino acid sequence (e.g., about 1%, 2%, 3%, 4%, 5%, 6%, 7%, 8%, 9%, 10% or more) are substituted with different amino acids with highly similar properties, are also readily identified as being highly similar to a disclosed construct. Such conservative variations of each disclosed sequence are also contemplated.

Methods for obtaining conservative variants, as well as more divergent versions of the polynucleotide and amino acid sequences, are widely known in the art. In addition to naturally occurring homologues which can be obtained, e.g., by screening genomic or expression libraries according to any of a variety of well-established protocols, see, e.g., Ausubel et al. Current Protocols in Molecular Biology (supplemented through 2004) John Wiley & Sons, New York (“Ausubel”); Sambrook et al. Molecular Cloning—A Laboratory Manual (2nd Ed.), Vol. 1-3, Cold Spring Harbor Laboratory, Cold Spring Harbor, N.Y., 1989 (“Sambrook”), and Berger and Kimmel Guide to Molecular Cloning Techniques, Methods in Enzymology volume 152 Academic Press, Inc., San Diego, Calif. (“Berger”), additional variants can be produced by any of a variety of mutagenesis procedures. Many such procedures are known in the art, including site directed mutagenesis, oligonucleotide-directed mutagenesis, and many others. For example, site directed mutagenesis is described, e.g., in Smith (1985) “In vitro mutagenesis” Ann. Rev. Genet. 19:423-462, and references therein, Botstein & Shortle (1985) “Strategies and applications of in vitro mutagenesis” Science 229:1193-1201; and Carter (1986) “Site-directed mutagenesis” Biochem. J. 237:1-7. Oligonucleotide-directed mutagenesis is described, e.g., in Zoller & Smith (1982) “Oligonucleotide-directed mutagenesis using M13-derived vectors: an efficient and general procedure for the production of point mutations in any DNA fragment” Nucleic Acids Res. 10:6487-6500). Mutagenesis using modified bases is described e.g., in Kunkel (1985) “Rapid and efficient site-specific mutagenesis without phenotypic selection” Proc. Natl. Acad. Sci. USA 82:488-492, and Taylor et al. (1985) “The rapid generation of oligonucleotide-directed mutations at high frequency using phosphorothioate-modified DNA” Nucl. Acids Res. 13: 8765-8787. Mutagenesis using gapped duplex DNA is described, e.g., in Kramer et al. (1984) “The gapped duplex DNA approach to oligonucleotide-directed mutation construction” Nucl. Acids Res. 12: 9441-9460). Point mismatch mutagenesis is described, e.g., by Kramer et al. (1984) “Point Mismatch Repair” Cell 38:879-887). Double-strand break mutagenesis is described, e.g., in Mandecki (1986) “Oligonucleotide-directed double-strand break repair in plasmids of Escherichia coli: a method for site-specific mutagenesis” Proc. Natl. Acad. Sci. USA, 83:7177-7181, and in Arnold (1993) “Protein engineering for unusual environments” Current Opinion in Biotechnology 4:450-455). Mutagenesis using repair-deficient host strains is described, e.g., in Carter et al. (1985) “Improved oligonucleotide site-directed mutagenesis using M13 vectors” Nucl. Acids Res. 13: 4431-4443. Mutagenesis by total gene synthesis is described e.g., by Nambiar et al. (1984) “Total synthesis and cloning of a gene coding for the ribonuclease S protein” Science 223: 1299-1301. DNA shuffling is described, e.g., by Stemmer (1994) “Rapid evolution of a protein in vitro by DNA shuffling” Nature 370:389-391, and Stemmer (1994) “DNA shuffling by random fragmentation and reassembly: In vitro recombination for molecular evolution,” Proc. Natl. Acad. Sci. USA 91:10747-10751.

Many of the above methods are further described in Methods in Enzymology Volume 154, which also describes useful controls for trouble-shooting problems with various mutagenesis methods. Kits for mutagenesis, library construction and other diversity generation methods are also commercially available. For example, kits are available from, e.g., Amersham International plc (Piscataway, N.J.) (e.g., using the Eckstein method above), Bio/Can Scientific (Mississauga, Ontario, CANADA), Bio-Rad (Hercules, Calif.) (e.g., using the Kunkel method described above), Boehringer Mannheim Corp. (Ridgefield, Conn.), Clonetech Laboratories of BD Biosciences (Palo Alto, Calif.), DNA Technologies (Gaithersburg, Md.), Epicentre Technologies (Madison, Wis.) (e.g., the 5 prime 3 prime kit); Genpak Inc. (Stony Brook, N.Y.), Lemargo Inc (Toronto, CANADA), Invitrogen Life Technologies (Carlsbad, Calif.), New England Biolabs (Beverly, Mass.), Pharmacia Biotech (Peapack, N.J.), Promega Corp. (Madison, Wis.), QBiogene (Carlsbad, Calif.), and Stratagene (La Jolla, Calif.) (e.g., QuickChange™ site-directed mutagenesis kit and Chameleon™ double-stranded, site-directed mutagenesis kit).

Determining Sequence Relationships

Similar sequences can be objectively determined by any number of methods, e.g., percent identity, hybridization, immunologically, and the like. A variety of methods for determining relationships between two or more sequences (e.g., identity, similarity and/or homology) are available and well-known in the art. Methods include manual alignment, computer assisted sequence alignment, and combinations thereof, for example. A number of algorithms (which are generally computer implemented) for performing sequence alignment are widely available or can be produced by one of skill. These methods include, e.g., the local homology algorithm of Smith and Waterman (1981) Adv. Appl. Math. 2:482; the homology alignment algorithm of Needleman and Wunsch (1970) J. Mol. Biol. 48:443; the search for similarity method of Pearson and Lipman (1988) Proc. Natl. Acad. Sci. (USA) 85:2444; and/or by computerized implementations of these algorithms (e.g., GAP, BESTFIT, FASTA, and TFASTA in the Wisconsin Genetics Software Package Release 7.0, Genetics Computer Group, 575 Science Dr., Madison, Wis.).

For example, software for performing sequence identity (and sequence similarity) analysis using the BLAST algorithm is described in Altschul et al. (1990) J. Mol. Biol. 215:403-410. This software is publicly available, e.g., through the National Center for Biotechnology Information on the internet at ncbi.nlm.nih.gov. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP (BLAST Protein) program uses as defaults a wordlength (W) of 3, an expectation (E) of 10, and the BLOSUM62 scoring matrix (see, Henikoff & Henikoff (1989) Proc. Natl. Acad. Sci. USA 89:10915).

Additionally, the BLAST algorithm performs a statistical analysis of the similarity between two sequences (see, e.g., Karlin & Altschul (1993) Proc. Nat'l. Acad. Sci. USA 90:5873-5787). One measure of similarity provided by the BLAST algorithm is the smallest sum probability (p(N)), which provides an indication of the probability by which a match between two nucleotide or amino acid sequences would occur by chance. For example, a nucleic acid is considered similar to a reference sequence (and, therefore, in this context, homologous) if the smallest sum probability in a comparison of the test nucleic acid to the reference nucleic acid is less than about 0.1, or less than about 0.01, and or even less than about 0.001.

Another example of a sequence alignment algorithm is PILEUP, which creates a multiple sequence alignment from a group of related sequences using progressive, pairwise alignments. It can also plot a tree showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS5:151-153. The program can align, e.g., up to 300 sequences of a maximum length of 5,000 letters. The multiple alignment procedure begins with the pairwise alignment of the two most similar sequences, producing a cluster of two aligned sequences. This cluster can then be aligned to the next most related sequence or cluster of aligned sequences. Two clusters of sequences can be aligned by a simple extension of the pairwise alignment of two individual sequences. The final alignment is achieved by a series of progressive, pairwise alignments. The program can also be used to plot a dendogram or tree representation of clustering relationships. The program is run by designating specific sequences and their amino acid or nucleotide coordinates for regions of sequence comparison.

An additional example of an algorithm that is suitable for multiple DNA, or amino acid, sequence alignments is the CLUSTALW program (Thompson, J. D. et al. (1994) Nucl. Acids. Res. 22: 4673-4680). CLUSTALW performs multiple pairwise comparisons between groups of sequences and assembles them into a multiple alignment based on homology. Gap open and Gap extension penalties can be, e.g., 10 and 0.05 respectively. For amino acid alignments, the BLOSUM algorithm can be used as a protein weight matrix. See, e.g., Henikoff and Henikoff (1992) Proc. Natl. Acad. Sci. USA 89: 10915-10919.

Polynucleotide hybridization similarity can also be evaluated by hybridization between single stranded (or single stranded regions of) nucleic acids with complementary or partially complementary polynucleotide sequences. Hybridization is a measure of the physical association between nucleic acids, typically, in solution, or with one of the nucleic acid strands immobilized on a solid support, e.g., a membrane, a bead, a chip, a filter, etc. Nucleic acid hybridization occurs based on a variety of well characterized physico-chemical forces, such as hydrogen bonding, solvent exclusion, base stacking, and the like. Numerous protocols for nucleic acid hybridization are well-known in the art. An extensive guide to the hybridization of nucleic acids is found in Tijssen (1993) Laboratory Techniques in Biochemistry and Molecular Biology—Hybridization with Nucleic Acid Probes, part I, chapter 2, “Overview of principles of hybridization and the strategy of nucleic acid probe assays,” (Elsevier, N.Y.), as well as in Ausubel et al. Current Protocols in Molecular Biology (supplemented through 2004) John Wiley & Sons, New York (“Ausubel”); Sambrook et al. Molecular Cloning—A Laboratory Manual (2nd Ed.), Vol. 1-3, Cold Spring Harbor Laboratory, Cold Spring Harbor, N.Y., 1989 (“Sambrook”), and Berger and Kimmel Guide to Molecular Cloning Techniques, Methods in Enzymology volume 152 Academic Press, Inc., San Diego, Calif. (“Berger”). Hames and Higgins (1995) Gene Probes 1, IRL Press at Oxford University Press, Oxford, England (Hames and Higgins 1) and Hames and Higgins (1995) Gene Probes 2, IRL Press at Oxford University Press, Oxford, England (Hames and Higgins 2) provide details on the synthesis, labeling, detection and quantification of DNA and RNA, including oligonucleotides.

Conditions suitable for obtaining hybridization, including differential hybridization, are selected according to the theoretical melting temperature (Tm) between complementary and partially complementary nucleic acids. Under a given set of conditions, e.g., solvent composition, ionic strength, etc., the. Tm is the temperature at which the duplex between the hybridizing nucleic acid strands is 50% denatured. That is, the Tm corresponds to the temperature corresponding to the midpoint in transition from helix to random coil; it depends on the length of the polynucleotides, nucleotide composition, and ionic strength, for long stretches of nucleotides.

After hybridization, unhybridized nucleic acids can be removed by a series of washes, the stringency of which can be adjusted depending upon the desired results. Low stringency washing conditions (e.g., using higher salt and lower temperature) increase sensitivity, but can product nonspecific hybridization signals and high background signals. Higher stringency conditions (e.g., using lower salt and higher temperature that is closer to the T.sub.m) lower the background signal, typically with primarily the specific signal remaining, See, also, Rapley, R. and Walker, J. M. eds., Molecular Biomethods Handbook (Humana Press, Inc. 1998).

“Stringent hybridization wash conditions” or “stringent conditions” in the context of nucleic acid hybridization experiments, such as Southern and northern hybridizations, are sequence dependent, and are different under different environmental parameters. An extensive guide to the hybridization of nucleic acids is found in Tijssen (1993), supra, and in Hames and Higgins 1 and Hames and Higgins 2, supra.

An example of stringent hybridization conditions for hybridization of complementary nucleic acids which have more than 100 complementary residues on a filter in a Southern or northern blot is 2×SSC, 50% formamide at 42° C., with the hybridization being carried out overnight (e.g., for approximately 20 hours). An example of stringent wash conditions is a 0.2×SSC wash at 65° C. for 15 minutes (see Sambrook, supra for a description of SSC buffer). Often, the wash determining the stringency is preceded by a low stringency wash to remove signal due to residual unhybridized probe. An example low stringency wash is 2×SSC at room temperature (e.g., 20° C. for 15 minutes).

In general, a signal to noise ratio of at least 2.5×-5× (and typically higher) than that observed for an unrelated probe in the particular hybridization assay indicates detection of a specific hybridization. Detection of at least stringent hybridization between two sequences indicates relatively strong structural similarity to those provided in the sequence listings herein.

Generally, “highly stringent” hybridization and wash conditions are selected to be about 5° C. or less lower than the thermal melting point (Tm) for the specific sequence at a defined ionic strength and pH (as noted below, highly stringent conditions can also be referred to in comparative terms). Target sequences that are closely related or identical to the nucleotide sequence of interest (e.g., “probe”) can be identified under stringent or highly stringent conditions. Lower stringency conditions are appropriate for sequences that are less complementary.

For example, in determining stringent or highly stringent hybridization (or even more stringent hybridization) and wash conditions, the stringency of the hybridization and wash conditions is gradually increased (e.g., by increasing temperature, decreasing salt concentration, increasing detergent concentration, and/or increasing the concentration of organic solvents, such as formamide, in the hybridization or wash), until a selected set of criteria are met. For example, the stringency of the hybridization and wash conditions is gradually increased until a probe comprising one or more of the present polynucleotide sequences, or a subsequence thereof, and/or complementary polynucleotide sequences thereof, binds to a perfectly matched complementary target, with a signal to noise ratio that is at least 2.5×, and optionally 5×, or 10×, or 100× or more, as high as that observed for hybridization of the probe to an unmatched target, as desired.

Using subsequences derived from the nucleic acids listed in the sequence listing, target nucleic acids can be obtained; such target nucleic acids are also a feature of the current disclosure. For example, such target nucleic acids include sequences that hybridize under stringent conditions to an oligonucleotide probe that corresponds to a unique subsequence of any of the polynucleotides in the sequence listing, or a complementary sequence thereof; the probe optionally encodes a unique subsequence in any of the amino acid sequences of the sequence listing.

For example, hybridization conditions are chosen under which a target oligonucleotide that is perfectly complementary to the oligonucleotide probe hybridizes to the probe with at least about a 5-10× higher signal to noise ratio than for hybridization of the target oligonucleotide to a negative control non-complimentary nucleic acid. Higher ratios of signal to noise can be achieved by increasing the stringency of the hybridization conditions such that ratios of about 15×, 20×, 30×, 50× or more are obtained. The particular signal will depend on the label used in the relevant assay, e.g., a fluorescent label, a calorimetric label, a radioactive label, or the like.

Vectors, Promoters and Expression Systems

Polynucleotide sequences of the present disclosure can be in any of a variety of forms, e.g., expression cassettes, vectors, plasmids, viral particles, or linear nucleic acid sequences. For example, vectors, plasmids, cosmids, bacterial artificial chromosomes (BACs), YACs (yeast artificial chromosomes), phage, viruses and nucleic acid segments can comprise the present nucleic acid sequences or subsequences thereof. These nucleic acid constructs can further include promoters, enhancers, polylinkers, regulatory genes, etc. Thus, the present disclosure also relates, e.g., to vectors comprising the polynucleotides disclosed herein, host cells that incorporate these vectors, and the production of the various disclosed polypeptides (including those in the sequence listing) by recombinant techniques.

In accordance with these aspects, the vector may be, for example, a plasmid vector, a single or double-stranded phage vector, or a single or double-stranded RNA or DNA viral vector. Such vectors may be introduced into cells as polynucleotides, preferably DNA, by well-known techniques for introducing DNA and RNA into cells. The vectors, in the case of phage and viral vectors, also may be and preferably are introduced into cells as packaged or encapsidated virus by well-known techniques for infection and transduction. Viral vectors may be replication competent or replication defective. In the latter case, viral propagation generally will occur only in complementing host cells.

In some examples, vectors include those useful for expression of polynucleotides and polypeptides of the present disclosure. Generally, such vectors comprise cis-acting control regions effective for expression in a host, operably linked to the polynucleotide to be expressed. Appropriate trans-acting factors are supplied by the host, supplied by a complementing vector or supplied by the vector itself upon introduction into the host.

In certain examples in this regard, the vectors provide for protein expression. Such preferred expression may be inducible expression, temporally limited expression, or expression restricted to predominantly certain types of cells, or any combination of the above. Some embodiments of inducible vectors can be induced for expression by environmental factors that are easy to manipulate, such as temperature and nutrient additives. A variety of vectors suitable to this aspect, including constitutive and inducible expression vectors for use in prokaryotic and eukaryotic hosts, are well-known and employed routinely by those of skill in the art. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as rhabdoviruses, baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids and binaries used for Agrobacterium-mediated transformations.

Vectors can include a selectable marker and a reporter gene. For ease of obtaining sufficient quantities of vector, a bacterial origin that allows replication in E. coli can be used. The following vectors, which are commercially available, are provided by way of example. Among vectors preferred for use in bacteria are pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Useful plant binary vectors include BIN19 and its derivatives available from Clontech. These vectors are listed solely by way of illustration of the many commercially available and well-known vectors that are available to those of skill in the art. It will be appreciated that any other plasmid or vector suitable for, for example, introduction, maintenance, propagation or expression of one or more polynucleotides and/or polypeptides as provided in the present sequence listing, including variants thereof as described, in a host may be used.

In general, expression constructs will contain sites for transcription initiation and termination, and, in the transcribed region, a ribosome-binding site for translation when the construct encodes a polypeptide. The coding portion of the mature transcripts expressed by the constructs will include a translation-initiating AUG at the beginning and a termination codon appropriately positioned at the end of the polypeptide to be translated. In addition, the constructs may contain control regions that regulate as well as engender expression. Generally, in accordance with many commonly practiced procedures, such regions will operate by controlling transcription, such as transcription factors, repressor binding sites and termination signals, among others. For secretion of a translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Transcription of the DNA (e.g., encoding the polypeptides) of the present disclosure by higher eukaryotes may be increased by inserting an enhancer sequence into the vector. Enhancers are cis-acting elements of DNA, usually about from 10 to 300 by that act to increase transcriptional activity of a promoter in a given host cell-type. Examples of enhancers include the SV40 enhancer, which is located on the late side of the replication origin at by 100 to 270, the cytomegalovirus early promoter enhancer, the polyoma enhancer on the late side of the replication origin, and adenovirus enhancers. Additional enhancers useful in the disclosure to increase transcription of the introduced DNA segment, include, inter alia, viral enhancers like those within the 35S promoter, as shown by Odell et al., Plant Mol. Biol. 10:263-72 (1988), and an enhancer from an opine gene as described by Fromm et al., Plant Cell 1:977 (1989). The enhancer may affect the tissue-specificity and/or temporal specificity of expression of sequences included in the vector.

Termination regions also facilitate effective expression by ending transcription at appropriate points. Useful terminators include, but are not limited to, pinII (see An et al., Plant Cell 1(1):115-122 (1989)), glb1 (see Genbank Accession #L22345), gz (see gzw64a terminator, Genbank Accession #S78780), and the nos terminator from Agrobacterium. The termination region can be native with the promoter nucleotide sequence, can be native with the DNA sequence of interest, or can be derived from another source. For example, other convenient termination regions are available from the Ti-plasmid of A. tumefaciens, such as the octopine synthase and nopaline synthase termination regions. See also: Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. 1989) Nucleic Acids Res. 17:7891-7903; and Joshi et al. (1987) Nucleic Acid Res. 15:9627-9639.

Among known eukaryotic promoters suitable for generalized expression are the CMV immediate early promoter, the HSV thymidine kinase promoter, the early and late SV40 promoters, the promoters of retroviral LTRs, such as those of the Rous sarcoma virus (“RSV”), metallothionein promoters, such as the mouse metallothionein-I promoter and various plant promoters, such as globulin-1. The native promoters of the polynucleotide sequences listing in the sequence listing may also be used. Representatives of prokaryotic promoters include the phage lambda PL promoter, the E. coli lac, trp and tac promoters to name just a few of the well-known promoters.

Isolated or recombinant viruses, virus infected cells, or cells including one or more portions of the present polynucleotide sequences and/or expressing one or more portions of the present amino acid sequences are also contemplated.

A polynucleotide, optionally encoding the heterologous structural sequence of an amino acid sequence as disclosed, generally will be inserted into a vector using standard techniques so that it is operably linked to a promoter for expression. Operably linked, as used herein, includes reference to a functional linkage between a promoter and a second sequence, wherein the promoter sequence initiates and mediates transcription of the DNA corresponding to the second sequence. Generally, operably linked means that the polynucleotide sequence being linked is contiguous and, where necessary to join two protein coding regions, contiguous and in the same reading frame. When the polynucleotide is intended for expression of a polypeptide, the polynucleotide will be positioned so that the transcription start site is located appropriately 5′ to a ribosome binding site. The ribosome-binding site will be 5′ to the AUG that initiates translation of the polypeptide to be expressed. Generally, there will be no other open reading frames that begin with an initiation codon, usually AUG, and lie between the ribosome binding site and the initiation codon. Also, generally, there will be a translation stop codon at the end of the polypeptide and there will be a polyadenylation signal in constructs for use in eukaryotic hosts. Transcription termination signals appropriately disposed at the 3′ end of the transcribed region may also be included in the polynucleotide construct.

For nucleic acid constructs designed to express a polypeptide, the expression cassettes can additionally contain 5′ leader sequences. Such leader sequences can act to enhance translation. Translation leaders are known in the art and include: picornavirus leaders, for example: EMCV leader (Encephalomyocarditis 5′ noncoding region), Elroy-Stein et al. (1989) Proc. Nat. Acad. Sci. USA 86:6126-6130; potyvirus leaders, for example, TEV leader (Tobacco Etch Virus), Allison et al. (1986); MDMV leader (Maize Dwarf Mosaic Virus), Virology 154:9-20; human immunoglobulin heavy-chain binding protein (BiP), Macejak et al. (1991) Nature 353:90-94; untranslated leader from the coat protein mRNA of alfalfa mosaic virus (AMV RNA 4), Jobling et al. (1987) Nature 325:622-625); tobacco mosaic virus leader (TMV), Gallie et al. (1989) Molecular Biology of RNA, pages 237-256; and maize chlorotic mottle virus leader (MCMV) Lommel et al. (1991) Virology 81:382-385. See also Della-Cioppa et al. (1987) Plant Physiology 84:965-968. The cassette can also contain sequences that enhance translation and/or mRNA stability such as introns. The expression cassette can also include, at the 3′ terminus of the isolated nucleotide sequence of interest, a translational termination region.

In those instances where it is desirable to have the expressed product of the polynucleotide sequence directed to a particular organelle or secreted at the cell's surface the expression cassette can further comprise a coding sequence for a transit peptide. Such transit peptides are well-known in the art and include, but are not limited to: the transit peptide for the acyl carrier protein, the small subunit of RUBISCO, plant EPSP synthase, and the like.

In making an expression cassette, the various DNA fragments can be manipulated so as to provide for the polynucleotide sequences in the proper orientation and, as appropriate, in the proper reading frame. Toward this end, adapters or linkers can be employed to join DNA fragments or other manipulations can be involved to provide for convenient restriction sites, removal of superfluous DNA, removal of restriction sites, or the like. For this purpose, in vitro mutagenesis, primer repair, restriction digests, annealing, and resubstitutions such as transitions and transversions, can be employed.

Introduction of a construct into a host cell can be effected by calcium phosphate transfection, DEAE-dextran mediated transfection, microinjection, cationic lipid-mediated transfection, electroporation, transduction, scrape loading, ballistic introduction, infection or other methods. Such methods are described in many standard laboratory manuals, such as Davis et al., Basic Methods in Molecular Biology, (1986) and Sambrook et al., Molecular Cloning—A Laboratory Manual, 2nd Ed., Cold Spring Harbor Laboratory Press, Cold Spring Harbor, N.Y. (1989).

Representative examples of appropriate hosts include bacterial cells, such as streptococci, staphylococci, E. coli, streptomyces and Salmonella typhimurium cells; fungal cells, such as yeast cells and Aspergillus cells; insect cells such as Drosophila S2 and Spodoptera Sf9 cells; animal cells such as CHO, COS and Bowes melanoma cells; and plant cells.

The host cells can be cultured in conventional nutrient media, which may be modified as appropriate for, inter alia, activating promoters, selecting transformants or amplifying genes. Culture conditions, such as temperature, pH and the like, previously used with the host cell selected for expression generally will be suitable for expression of nucleic acids and/or polypeptides, as will be apparent to those of skill in the art. Mature proteins can be expressed in mammalian cells, yeast, bacteria, or other cells under the control of appropriate promoters. Cell-free translation systems can also be employed to produce such proteins using RNAs derived from the polynucleotides disclosed herein.

Following transformation of a suitable host strain and growth of the host strain to an appropriate cell density, where the selected promoter is inducible it is induced by appropriate means (e.g., temperature shift or exposure to chemical inducer) and cells are cultured for an additional period. Cells typically then are harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents; such methods are well-known to those skilled in the art.

Compositions and methods of the present disclosure can include administering the polynucleotides and/or amino acids as provided herein. For example, treatments for glioblastoma can include administering one or more of the polynucleotides and/or amino acids. The one or more polynucleotides and/or amino acids may be in an isolated form or may be part of a composition, including a viral particle. In various embodiments, the administering can take the following forms: intradermal, transdermal, parenteral, intravascular, intravenous, intramuscular, intranasal, subcutaneous, regional, percutaneous, intratracheal, intraperitoneal, intraarterial, intravesical, intratumoral, inhalation, perfusion, lavage, direct injection, alimentary, oral, or intracranial administration. The mode of administration may depend on

### EXAMPLES

### Example 1

### Identification of Non-Neurotoxic Rhabdoviruses and In Vitro Cytotoxicity

To determine in vivo neurotoxicity: groups of 6-8 weeks old female BALB//c mice (n=3/group) received a single intracranial (IC) injection of the indicated viruses at 1e7 pfu. Following IC injection, mice were monitored daily for signs of distress including weight loss, piloerection, hind-limb paralysis and respiratory distress.

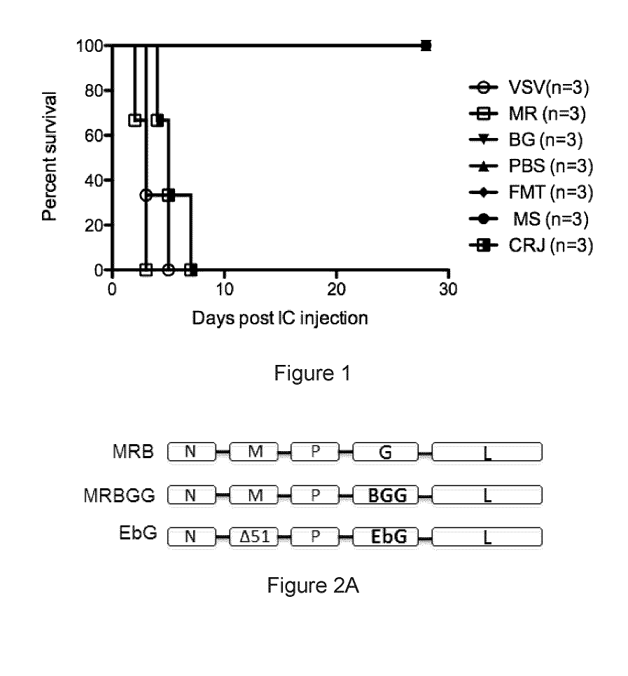

FIG. 1 shows the survival of BALB/c mice after a single IC dose of the indicated virus (1e7 pfu). Animals treated with IC injection of VSV, Maraba Virus (MR) or Carajas Virus (CRJ) survived less than 10 days while control animals (PBS) and all other animals injected IC with Farmington (FMT), Bahia Grande (BG) and Muir Springs (MS) showed 100% survival out to 30 days post IC injection indicative of their non-neurotoxic potential. Kaplan Meier survival plots were compared using Mantel-Cox Log rank analysis (Graphpad Prism).

In addition to exploring the oncolytic potential of wild-type FMT, BG and MS, the authors of the present disclosure reasoned that generating chimeric viruses of maraba virus (MRB) and a non-neurotoxic virus (for example, BG) would result in a virus with both desirable properties. The glycoprotein from BG was swapped into MRB, creating a chimeric Maraba virus with BG glycoprotein, termed “Maraba BGG” or “MRBGG” or “MRB-BG(G) or variations thereof, and including the RNA sequence which is the reverse complement of SEQ ID NO: 10. Rhabdoviruses, such as Maraba virus, carry their genetic material in the form of negative-sense single-stranded RNA. The RNA sequences disclosed herein correspond to RNA strands which encode the viral genetic material and are, therefore, the reverse complement of the genetic RNA which are carried by the rhabdoviruses.

The genome of the Maraba MGG viral particle has open reading frames that encode Maraba proteins N, P, and L; as well as Maraba protein M; and Bahia Grande G protein. The Maraba protein N has a sequence which corresponds to SEQ ID NO: 1. The Maraba protein P has a sequence which corresponds to SEQ ID NO: 2. The Maraba protein L has a sequence which corresponds to SEQ ID NO: 3. The Maraba protein M has a sequence which corresponds to SEQ ID NO: 4. The Bahia Grande G protein has a sequence which corresponds to SEQ ID NO: 6.

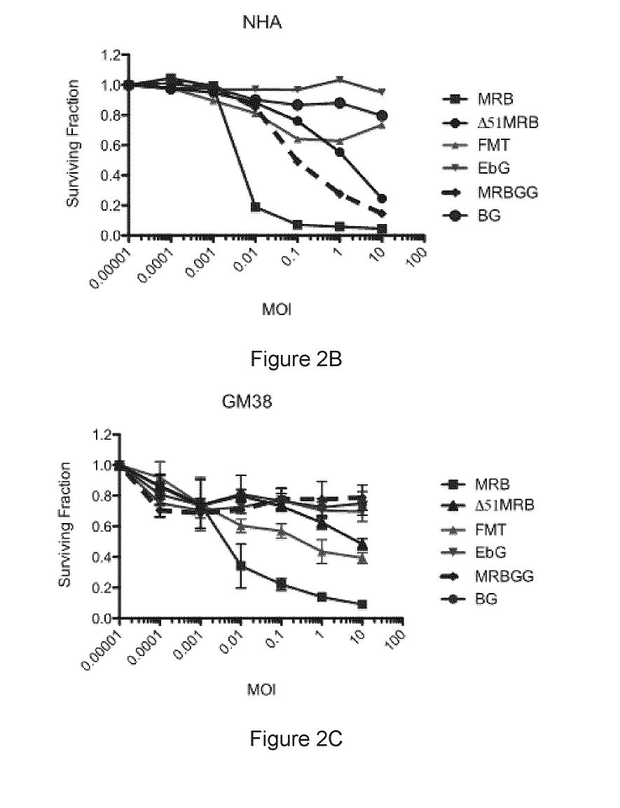

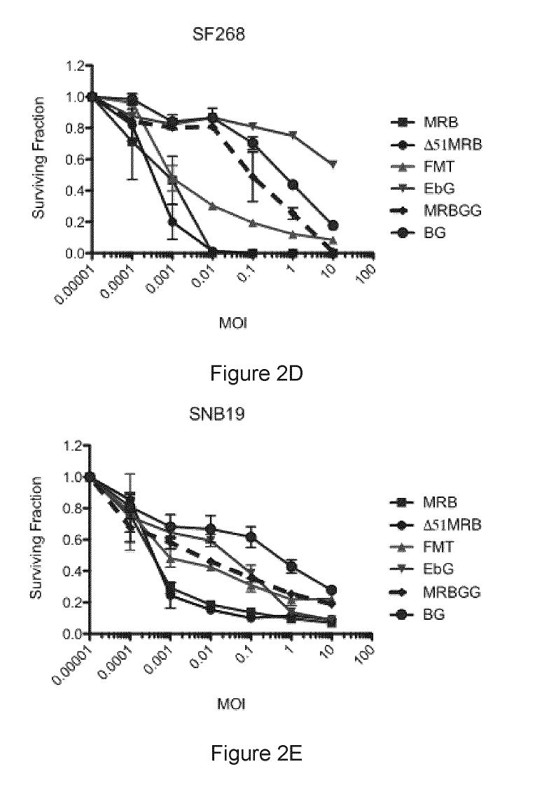

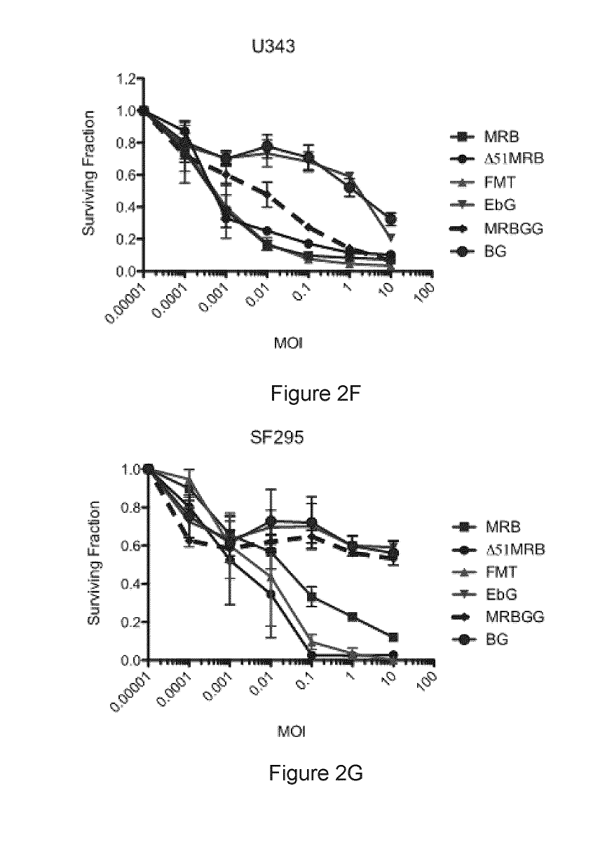

Another chimeric virus was produced by swapping out the MRB G glycoprotein for the Ebola glycoprotein, this time into the more attenuated Maraba vector (Δ051MRB) to create a chimeric virus, termed “Maraba EbG” or “EbG” or variations thereof, and including the RNA sequence which is the reverse complement of SEQ ID NO: 14 (see FIG. 2A). The genome of the Maraba EbG viral particle has open reading frames that encode Maraba proteins N, P, and L; as well as Maraba protein Δ051M; and Ebola G protein. The Maraba protein N has a sequence which corresponds to SEQ ID NO: 1. The Maraba protein P has a sequence which corresponds to SEQ ID NO: 2. The Maraba protein L has a sequence which corresponds to SEQ ID NO: 3. The Maraba protein Δ051M has a sequence which corresponds to SEQ ID NO: 5. The Ebola G protein has a sequence which corresponds to SEQ ID NO: 8.

The authors of the present disclosure hypothesized that the Maraba EbG variant would increase the therapeutic window for the chimeric virus in a replicating oncolytic rhabdovirus (FIG. 2A) since it has been previously demonstrate that a lentiviral vector pseudotyped with Ebola-Zaire glycoprotein resulted in no viral transduction of the mouse CNS while retaining the ability to transduce 293T cancer cell line (see Watson, D. J., Kobinger, G. P., Passini, M. A., Wilson, J. M. & Wolfe, J. H. Targeted transduction patterns in the mouse brain by lentivirus vectors pseudotyped with VSV, Ebola, Mokola, LCMV, or MuLV envelope proteins. Mol Ther 5, 528-537 (2002); and Watson, D. J., Passini, M. A. & Wolfe, J. H. Transduction of the choroid plexus and ependyma in neonatal mouse brain by vesicular stomatitis virus glycoprotein-pseudotyped lentivirus and adeno-associated virus type 5 vectors. Hum Gene Ther 16, 49-56 (2005)).

To test the killing capacity of these chimeric viruses, as compared to wild type isolates, cell killing assays were performed on 2 normal human diploid cell lines primary normal human astrocytes (NHA) and primary fibroblasts (GM38) (FIGS. 2B and 2C) and a panel of 8 CNS tumour cell lines SF268, SNB19, U118, U343, SF295, SNB75, SF539 and U373 (FIGS. 2D through 2K).

Cells were acquired from the National Institute of General Medical Science Mutant Cell Repository, Camden, N.J. and were propagated in Dulbecco's modified Eagle's medium (Hyclone, Logan, Utah) supplemented with 10% fetal calf serum (Cansera, Etobicoke, Ontario, Canada). Viability Assays were performed with the indicated cell lines as follows: Cells were plated at a density of 10 000 cells/well into 96 well plates and infected the next day with either: wild-type Maraba, wild type FMT, wild type BG, attenuated Maraba, Maraba EbG, or Maraba BGG at various multiplicity of infections (0.0001-10 pfu/cell).

Following a 48 hour incubation, Alamar Blue (Resazurin sodium salt (Sigma-Aldrich) was added to a final concentration of 20 μg/ml. After a 6 hour incubation the absorbance was read at a wavelength of 573 nm. While wild type Maraba was very potent against all of the GBM cell lines, it was also highly lytic against both NHA and GM38. In contrast, Maraba EbG and wild-type BG demonstrated significant selective killing of tumour cell lines at MOIs (10 pfu) that were innocuous to normal cells (NHA and GM38). The chimeric virus “MRBGG”, demonstrated greater potency than Maraba EbG or wild-type BG against the majority of GBM cell lines, while remaining very safe in normal fibroblasts. Wild type FMT demonstrated the greatest therapeutic index, with potency rivaling MRB in the majority of GBM lines while remaining highly attenuated in NHA and GM38 primary cell lines. This demonstrates that wild type FMT, and Maraba viruses engineered to be chimeric for BG or Ebola glycoproteins, show potent and selective oncolytic activity when tested against brain cancer cell lines.

### Example 2

### In Vivo Safety of two Maraba Virus Chimeras

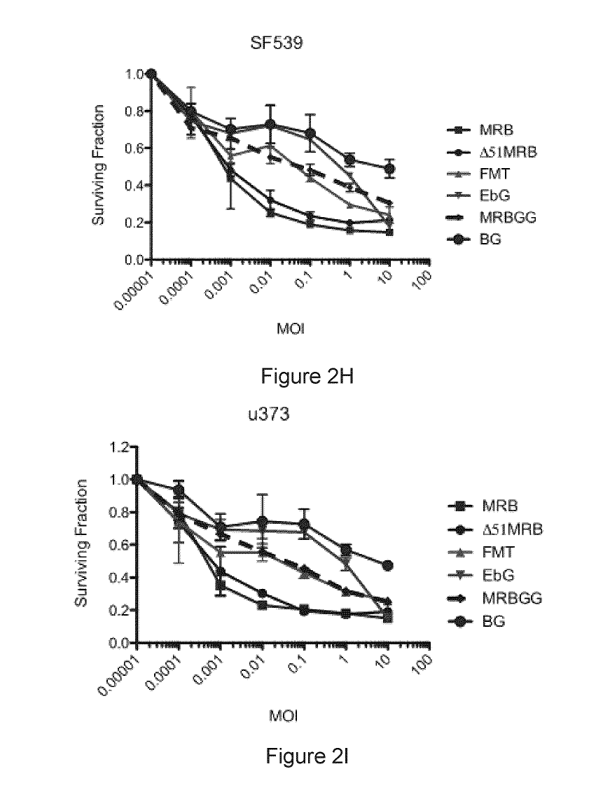

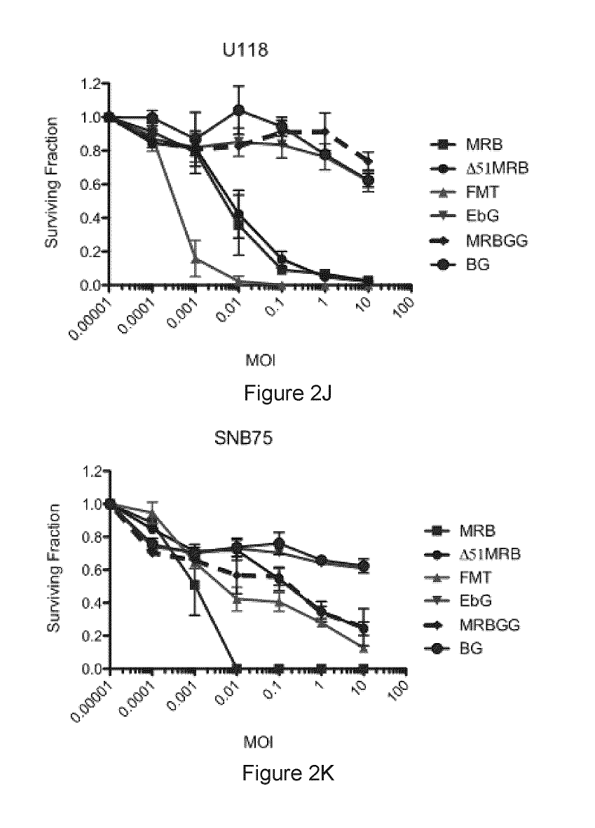

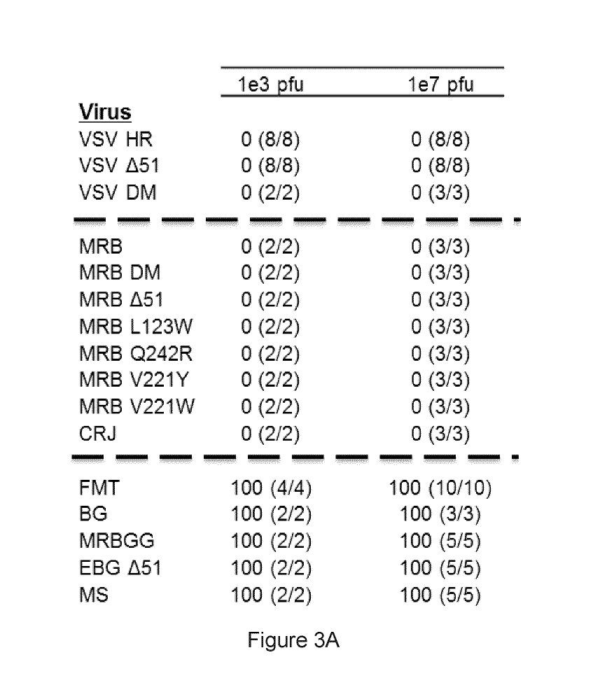

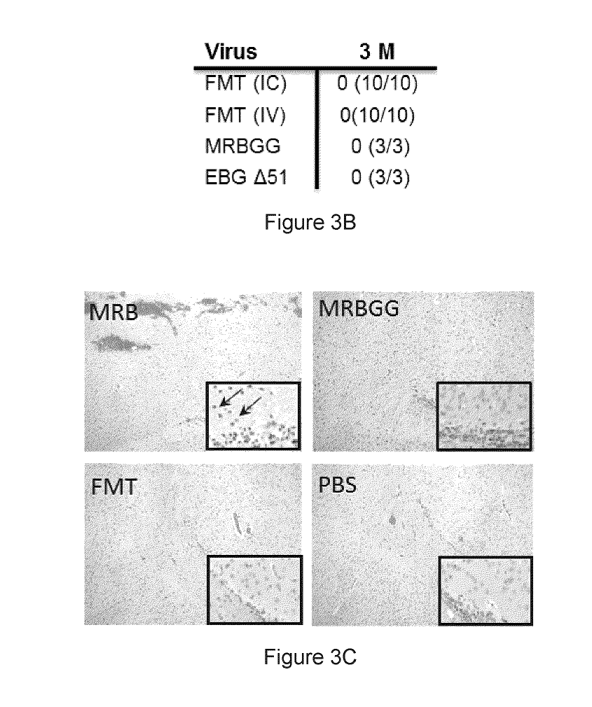

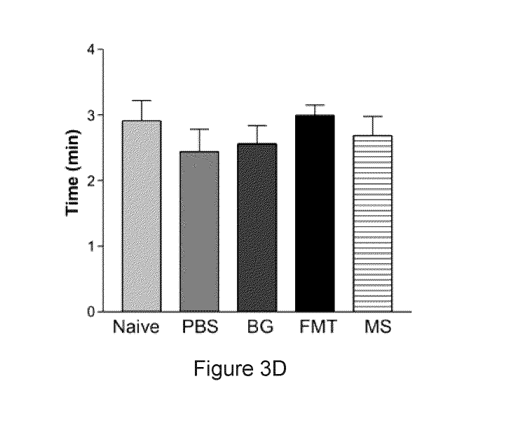

The wild type isolates (FMT, BG and MS) and the two chimeric viruses (EbG and MRBGG) which demonstrated attenuation in non-transformed cells in vitro (see Example 1), were tested to ascertain whether the observed attenuation translates to safety in vivo. Animals were administered two doses intracerebrally, a low (1e3 pfu) and high dose (1e7 pfu) of these viruses (FIG. 3A).

All 5 viruses were found to be safe, with 100% of the animals surviving 100 days post treatment with no persistent infection. At these doses, animals displayed transient weight loss and piloerection which is consistent with viral infection, but these symptoms resolved within 5-7 days post inoculation. In contrast, all animals that received similar IC doses of wild type or attenuated Maraba and VSV strains succumbed to infection within a week (FIG. 3A). These animals displayed clinical signs of a CNS infection with rapid and progressive weight loss, hind leg paralysis and had significant titres of virus in their brain just prior to death (data not shown).

Viral titres were determined by plaque assay on animal brains 3 months after treatment with wild type FMT (IC and IV) and the chimeric Maraba viruses (EbG and MRBGG). Plaque assays were performed with Vero cells plated at a density of 5e5 cells per/well of a 6 well dish. The next day 100 μl of serial viral dilutions were prepared and added for 1 hour to Vero cells. After viral adsorption, 2 ml of agarose overlay was added (1:1 1% agarose: 2×DMEM and 20% FCS) and plaques were counted the following day. No virus was detected in animal brains 3 months post IC infection (FIG. 3B).

In addition, following administration of high doses of FMT (1e7 pfu) and MRBGG (1e7 pfu) in the brain, no signs of cell death or inflammatory responses were found comparable to those of saline injected control mice (FIG. 3C). This differed dramatically from wild-type MRB injected animals, which displayed a striking increase in inflammatory cells, condensed nuclei, and a perforated morphology.

Although no acute neurotoxicity resulted from IC treatment with FMT, BG, or MS, an assessment of their cognitive and motor function was performed several days after virus infection. Motor function was assessed before and after treatment with these 3 wild type viruses (FIG. 3D). Balb/C mice were tested for motor function/performance on a rotating rod apparatus prior to IC viral administration. Mice were placed on a rotorod for 3 trials per day for 4 consecutive days. After allowing the animals 0.5 min to adjust to the apparatus, the rod was accelerated in a linear fashion at 0.1 rpm/s. Latency to fall was measured in minutes and animals were divided into groups of 3. Motor function was assessed one week post injection in Naïve (uninjected), PBS, FMT, Maraba EbG, BG, MRBGG and MS IC treated animals. Standard error of the mean was calculated. Specifically, there is no significant difference in the latency to fall between the mock-infected animals or virus infected animals, 1 week prior and 1 week post injection (FIG. 3D).

In addition to intracranial toxicity, the toxicity of FMT and MRBGG was evaluated when administered intravenously (IV) in immunocompetent mice with escalating doses of virus (FIG. 3E). MRBGG is tolerated up to a dose of 3e8 pfu, which demonstrates IV safety that is one order of magnitude safer than published results of wild type Maraba. FMT is well tolerated IV and never reaches an LD50 even at our highest dose 3e9 pfu which is comparable to an attenuated version of Maraba as previously described (Brun, J. et al. Identification of Genetically Modified Maraba Virus as an Oncolytic Rhabdovirus. Mol Ther 18, 1440 (2010)). FMT animals IV dosed at greater than 3e8 pfu displayed transient weight loss and moderate piloerection, which resolved 5-7 days post treatment (data not shown).

### Example 3

### In Vivo Efficacy of Maraba Virus Chimeras

The in vivo efficacy of chimeric Maraba viruses was also determined in mouse models of glioblastoma. The sensitivity of the human glioblastoma cell line U87MG to viral infection in vitro was determined. FMT and wild type Maraba were equally potent at killing U87MG cells with an EC50 score of less than 0.001 multiplicities of infection (data not shown). Maraba virus chimeras (Maraba EbG, Maraba BGG) and BG wild-type were also potent at killing U87MG cells in vitro with an EC50 score of less than 0.1 multiplicities of infection (data not shown).

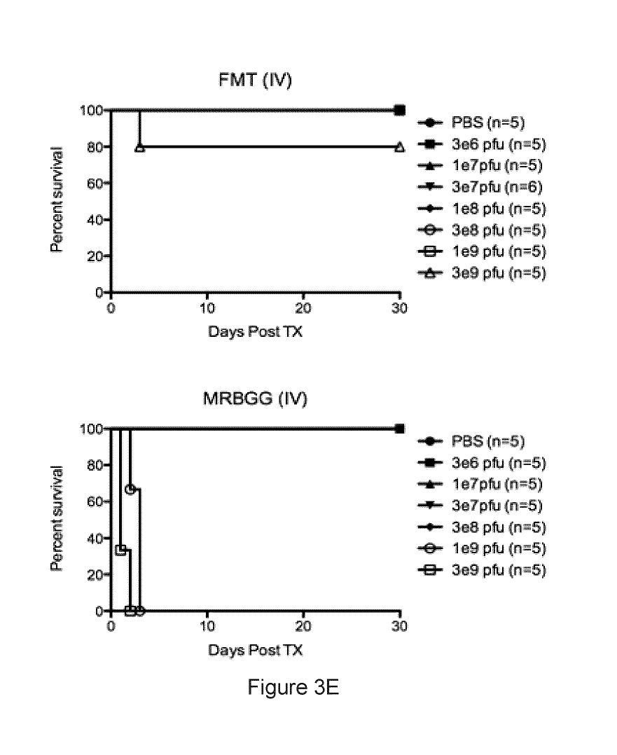

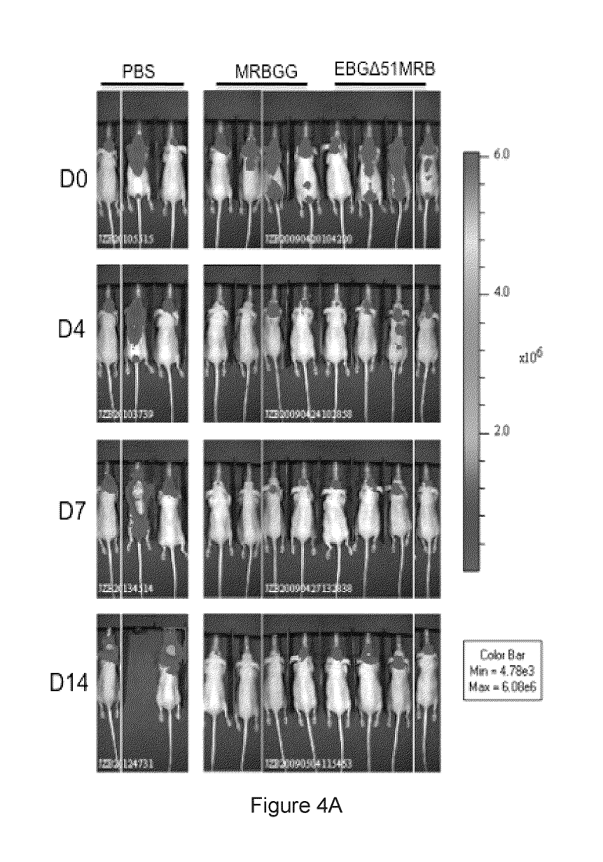

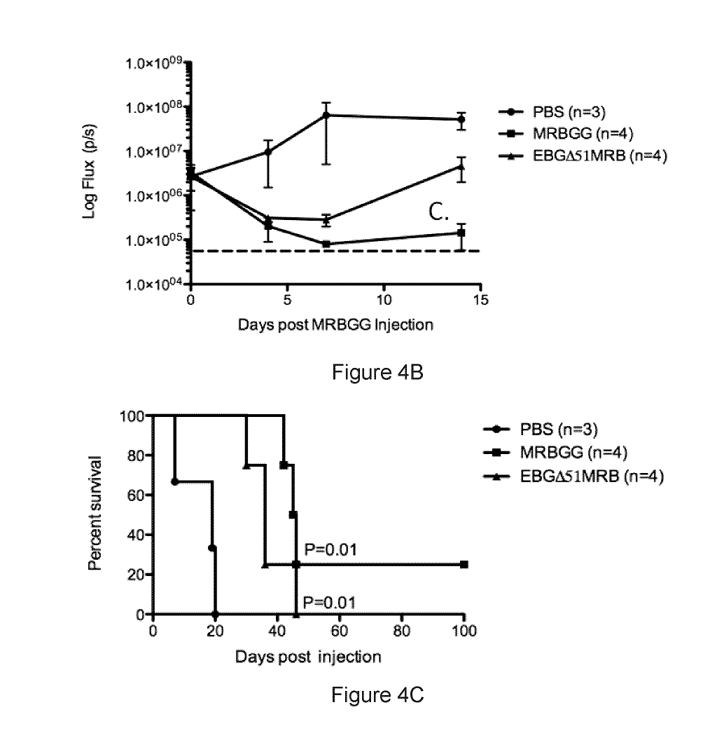

After adapting human U87MG glioma cells for bioluminescent imaging, an intracerebral U87MG glioma model in athymic mice was established and IV efficacy of Maraba virus chimeras according to the present disclosure was examined in this model (FIG. 4 A-C). In the human glioblastoma xenograft model human, glioblastoma U87MG cells were adapted for bioluminescent imaging by transducing with lentivirus containing firefly luciferase (FLUC) and transfecting FLUC plasmid respectively. U87MG FLUC cells were injected IC into CD1 nude mice. Untreated CD-1 animals develop tumours at about day 15-21.

Animals with FLUC expressing tumours were monitored for tumour progression using the live imaging IVIS Xenogen 200 system after an IP injection of luciferin (Gold Biotechnology Inc). The animals were monitored for signs of distress including survival, weight loss, morbidity, piloerection, hind-limb paralysis and respiratory distress. Three days after the first treatment a significant decrease in tumour burden was observed with a maximal effect observed by day 7 (FIGS. 4 A & B). However by day 14 tumors were starting to recur. Also observed was a delay in time to death following intravenous treatment with Maraba virus chimeras (FIG. 4C). Interestingly, the spinal metastases in Maraba virus chimera treated animals in this model are completely cleared in all tumour bearing animals. In contrast, animals treated with UV inactivated virus had a significant increase in tumour burden by day 7 at which point they started exhibiting neurological symptoms from their brain tumours. All IV treated animals responded to treatment with 3 of 8 durably cured and surviving beyond 100 days post treatment.

### Example 4

### Exploring Other Maraba Virus Chimeras

Vesicular stomatitis virus (VSV) is a potent oncolytic rhabdovirus. However, neurotropism with subsequent neurovirulence, as well as a highly potent nAb response are problems associated with VSV treatment. The inherent neurotoxicity has hindered its consideration as a clinical candidate.

The inherent neurotoxicity is thought to be mediated by its glycoprotein (VSV-G). However, lentiviral vectors that typically use VSV-G have had their neurotoxicity attenuated through pseudotyping with the lymphocytic choriomeningitis virus G protein (LCMV-G) (Beyer et al., J Virol 76:1488-1495, 2002; and U.S. Patent Publication No. 2011/0250188 to Von Laer). LCMV is a prototypical member of the arenavirus family of enveloped negative sense RNA viruses. The authors of the present disclosure hypothesized that the neurotoxicity of the Maraba virus may be attenuated through replacement of its glycoprotein (Maraba-G protein) with LCMV-G protein. A chimeric Maraba virus having LCMV-G protein was produced by swapping out the MRB G glycoprotein for the LCMV glycoprotein to create a chimeric virus, termed “Maraba LCMV-G” or “Maraba LCMV(G)”, and including the RNA sequence which is the reverse complement of SEQ ID NO: 12 (see FIG. 2A).

The genome of the Maraba LCMV-G viral particle has open reading frames that encode Maraba proteins N, P, and L; as well as Maraba protein M; and LCMV-G protein. The Maraba protein N has a sequence which corresponds to SEQ ID NO: 1. The Maraba protein P has a sequence which corresponds to SEQ ID NO: 2. The Maraba protein L has a sequence which corresponds to SEQ ID NO: 3. The Maraba protein M has a sequence which corresponds to SEQ ID NO: 4. The LCMV-G protein has a sequence which corresponds to SEQ ID NO: 7.

Manufacturing and rescuing Maraba chimeric viruses was performed as follows: A plasmid encoding the wildtype recombinant Maraba virus genome (Brun et al., 2010) was modified by standard DNA cloning methods so that the Maraba glycoprotein sequence was replaced with the glycoprotein sequences from Bahia Grande Virus, Leukocytic Choriomenigitis Virus (LCMV) or Farmington Virus. Briefly, a NotI restriction site was introduced by PCR-based mutageneis directly after the stop codon in the Maraba G sequence. Using this newly introduced NotI site and existing KpnI site between the M and G protein sequences, Maraba G was removed by restriction digest to generate pMRB(-G)-KpnI/NotI. Primers to amplify the glycoprotein sequences of both Farmington and Bahia Grande were designed to introduce 5′ KpnI and 3′ NotI restriction sites. These sequences were amplified by PCR and ligated into pMRB(-G)-KpnI/NotI. The LCMV glycoprotein precursor sequence (GenBank EF164923.1) was synthesized with 5′ KpnI and 3′ NotI sites introduced (Integrated DNA Technologies, Coralville, Iowa). This DNA fragment was ligated into the above-described pMRB(-G)-KpnI/NotI, becoming pMRB-LCMV-G, pMRB-BG-G and pMRB-FMT-G.

Additionally, the recombinant genome of the Farmington Virus was modified, replacing wild-type Farmington glycoprotein with the Maraba glycoprotein, as described in PCT Application No. PCT/CA2012/050385 and in a similar manner to creating the Maraba glycoprotein variants described above.

Recombinant Maraba virus particles [MRBGG, MRB FMTG, MRB LCMVG] were generated using techniques described previously (Brun et al., 2010) from the modified Maraba genomic plasmids described above. Briefly, A549 cells were infected at an MOI of 10 with T7 RNA polymerase-expressing vaccinia virus for 1.5 h. Cells were subsequently transfected by lipofectamine 2000 with above-described modified recombinant Maraba genomic plasmids together with pCl-Neo constructs encoding the Maraba N, P and L proteins. Forty-eight hours after transfection the media was removed, filtered through a 0.2 μm filter and the filtrate used to infect SNB19 cells. Cytopathic effect was observed in successful rescues after forty-eight hours and the virus was then plaque purified three times on Vero cells. FMT-MRB-G virus was generated in a similar fashion as above except that the initial transfection contained pFMT-MRB-G and PCI-Neo constructs encoding Farmington N, P and L proteins.

Recombinant viruses underwent three rounds of plaque purification (on SNB19 cells), before scale up, purification on sucrose cushion, and resuspension in PBS containing 15% glucose.

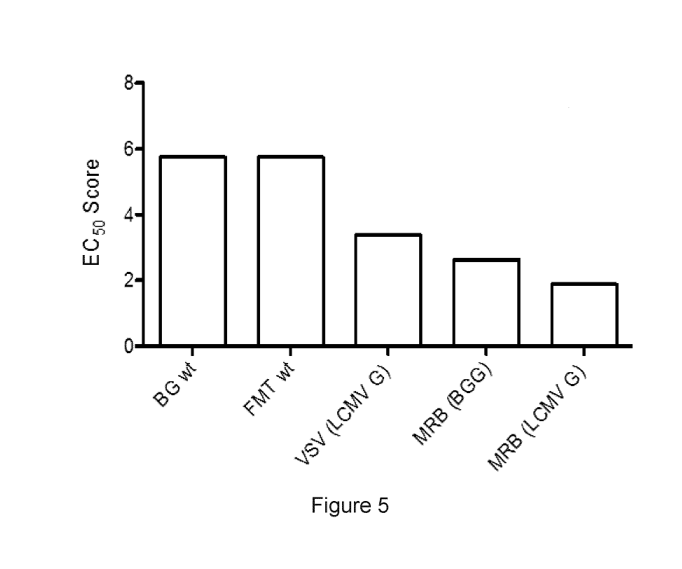

The relative cytotoxicity of a variety of viruses on a panel of human glioblastoma (Astrocytoma) cells (U87MG, SF268, U118, U373, U343, SNB19, 2 primary patient GBM cell samples) was determined. The indicated cell lines were seeded into 96 well plates (1e4 cells/well). The next day cells were infected with the indicated viruses: wild type BG, wild type FMT, VSV LCMVG (“VSV (LCMV G)”), MRB BGG (“MRB (BGG)”), or MRB LCMVG (“MRB (LCMV G)”) at various MOIs (0.0001-10 pfu/cell). Following a 96 hour incubation, Alamar Blue (Resazurin sodium salt (Sigma-Aldrich)) was added to a final concentration of 20 μg/ml. After a 6 hour incubation the absorbance was read at a wavelength of 573 nm. Cell metabolic viability was plotted and the multiplicity of infection (MOI) EC50 values were determined and then scored in ranges as follows: 1=MOI<0.01; 2=MOI<0.1; 3=MOI<1; 4=MOI<10; 5=MOI>10; 6=resistant. The average of the EC50 score for all 8 glioma lines was plotted for each virus (FIG. 5). The MRB-LCMVG chimera displayed the lowest EC50 value (and therefore highest potency with respect to oncolytic activity against brain cancer cell lines) versus MRBBG and VSV-LCMVG chimeras or wild type non-neurotoxic BG and FMT viruses.

### Example 5

### In Vivo Safety of Other Rhabdovirus Chimeras

To determine in vivo neurotoxicity: groups of 6-8 weeks old female BALB//c mice (n=2 to 10/group) received a single intracranial (IC) injection of the indicated viruses at 1e7 pfu. After administration of general anaesthetic (isoflurane), mice were prepared for surgery by shaving heads, applying chlorhexidine disinfectant to scalp, covering eyes with antibiotic ointment and applying a topical anaesthetic to ears. Mice were then placed onto a stereotaxic mount and immobilized using ear bars. With a scalpel blade, a 0.5 cm incision down the midline of the scalp was made to expose the top of the skull. Using a disposable 23 G needle, a hole on the right side of the skull, approximately 0.5 mm above the coronal suture and 2 mm from the sagittal suture, was made. A 10 μL glass Hamilton syringe was loaded with virus diluted in phosphate buffered saline (PBS) and mounted on the stereotaxic syringe pump. The needle was inserted to a depth of 4 mm and after 30 seconds was withdrawn by 0.5 mm. The virus (dose 1e7 pfu) was then infused into the brain at a rate of 5 μL/minute. After a subsequent 30 second wait time, the needle was withdrawn, the scalp glued together with veterinary adhesive and the animal was allowed to recover from general anaesthetic in an infant incubator. Mice received follow-up pain control (buprenorphine) for 72 h post surgery during which time body mass was measured and wellness assessments were made every 12 h.

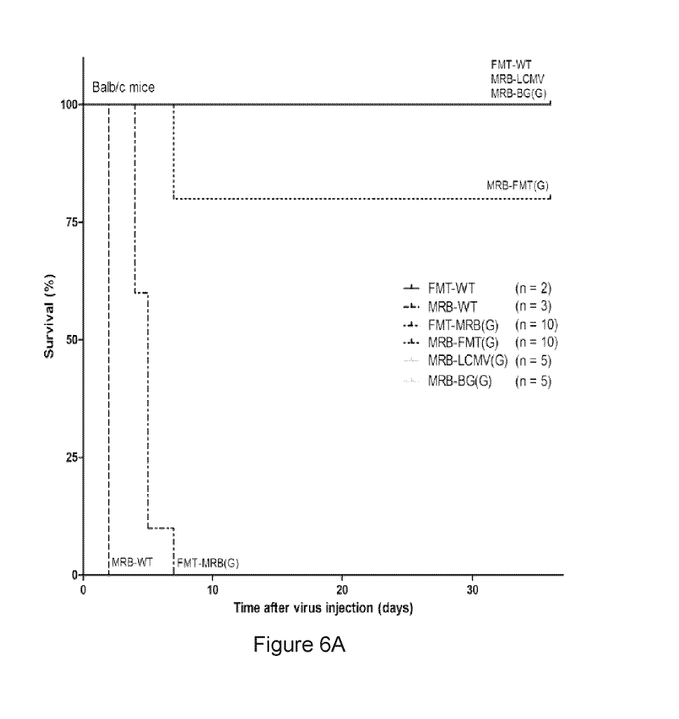

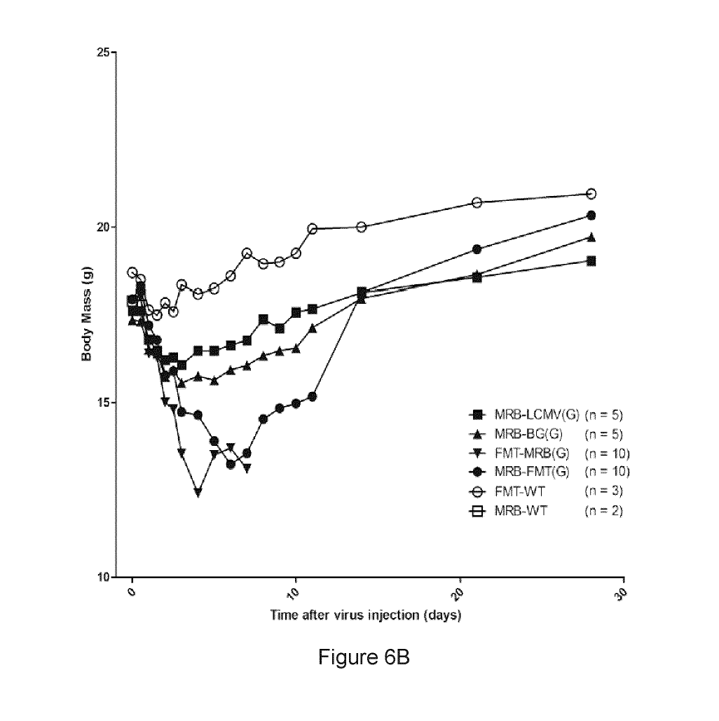

FIG. 6A shows Kaplan Meier survival plots of BALB/c mice after a single IC dose of the indicated virus (1e7 pfu). The survival plots were compared using Mantel-Cox Log rank analysis (Graphpad Prism). Animals treated with IC injection of wild-type Maraba Virus (MRB-WT) or chimeric Farmington virus having Maraba-G protein (FMT-MRB(G)) survived less than 10 days while animals injected IC with wild-type Farmington (FMT-WT), Maraba LCMV-G, and Maraba BGG showed 100% survival out to 30 days post IC injection indicative of their non-neurotoxic potential. Chimeric Maraba virus having Farmington G protein (MRB-FMT(G)) showed less than 100% survival at 30 days post IC injection, but increased survival vs. control. Animals treated with chimera MRB-FMT(G) showed an intermediate survival rate due to two mice being euthanized early due to loss of body mass.

The MRB-FMT(G) viral particle produces a cDNA polynucleotide which includes SEQ ID NO: 15 when the virus is in a host cell. The MRB-FMT(G) viral particle includes the RNA sequence which is the reverse complement of SEQ ID NO: 16. The genome of the MRB-FMT(G) virus has open reading frames that encode Maraba proteins N, P, and L; as well as Maraba protein M; and Farmington G protein. The Maraba protein N has a sequence which corresponds to SEQ ID NO: 1. The Maraba protein P has a sequence which corresponds to SEQ ID NO: 2. The Maraba protein L has a sequence which corresponds to SEQ ID NO: 3. The Maraba protein M has a sequence which corresponds to SEQ ID NO: 4. The Farmington-G protein has a sequence which corresponds to SEQ ID NO: 17.

The FMT-MRB(G) viral particle produces a cDNA polynucleotide which includes SEQ ID NO: 18 when the virus is in a host cell. The FMT-MRB(G) viral particle includes the RNA sequence which is the reverse complement of SEQ ID NO: 19. The genome of the FMT-MRB(G) virus has open reading frames that encode Farmington proteins N, P, and L; as well as Farmington protein M; and Maraba G protein. The Farmington protein N has a sequence which corresponds to SEQ ID NO: 20. The Farmington protein P has a sequence which corresponds to SEQ ID NO: 21. The Farmington protein L has a sequence which corresponds to SEQ ID NO: 22. The Farmington protein M has a sequence which corresponds to SEQ ID NO: 23. The Maraba-G protein has a sequence which corresponds to SEQ ID NO: 24.

FIG. 6B shows the corresponding body mass variations. All animals showed an initial drop in body mass 3-5 days after treatment. In animals treated with an IC injection of wild type FMT, or chimeras MRBGG or MRB LCMVG the drop in body mass was temporary and animals recovered initial body mass between 20-25 days following treatment. The three animals that remained from the group treated with chimera MRB FMTG showed a moderate recovery of body mass in the same time period.

FIGS. 6A and 6B indicate (i) that Farmington virus, a non-neurotoxic virus, may be made neurotoxic by replacement of its G-protein with the wild type G-protein from Maraba virus, a neurotoxic virus; and (ii) Maraba virus, a neurotoxic virus, is not made non-neurotoxic by replacement of its G-protein with any G-protein from a non-neurotoxic virus since replacement with the G-protein from the Farmington virus did not confer non-neurotoxicity (to be clear, Maraba virus is made non-neurotoxic by replacement of its G-protein with specific non-neurotoxic G-proteins).

### Example 6

### In Vivo Efficacy of Maraba Chimeric Viruses According to the Present Disclosure and Control Viruses

The in vivo efficacy of chimeric viruses was also determined in mouse models of glioblastoma. Six to eight week old CD-1 nude mice were injected intracranially with 1e6 U87MG-Fluc cells (human glioblastoma cells transduced with lentivirus to express firefly luciferase), as described above. One week later, mice were imaged using an in-vivo imaging system (Xenogen IVIS 200 Imaging System, Caliper Life Sciences) and sorted so that groups of five had similar levels of firefly luciferase expression from the established tumours in their brains. Briefly, mice were anaesthetized using isoflurane, injected with luciferin solution (2 mg/mouse) and placed into the IVIS machine. Images were taken and luminescence quantified using manufacturers' software (Living Image®, Caliper Life Sciences). The tumour signal from each mouse was normalized to the background signal from that exposure. This pre-treatment value was assigned a value of 100% and all subsequent values were compared to this starting point. The next day, mice were again stereotaxically injected with the indicated virus (dose 1e7 pfu, or phosphate buffered saline as a control), as described previously. Mice were imaged by IVIS at one week intervals for five weeks and during this time, as tumour-related health indicators warranted, mice were humanely euthanized as per institutional guidelines.

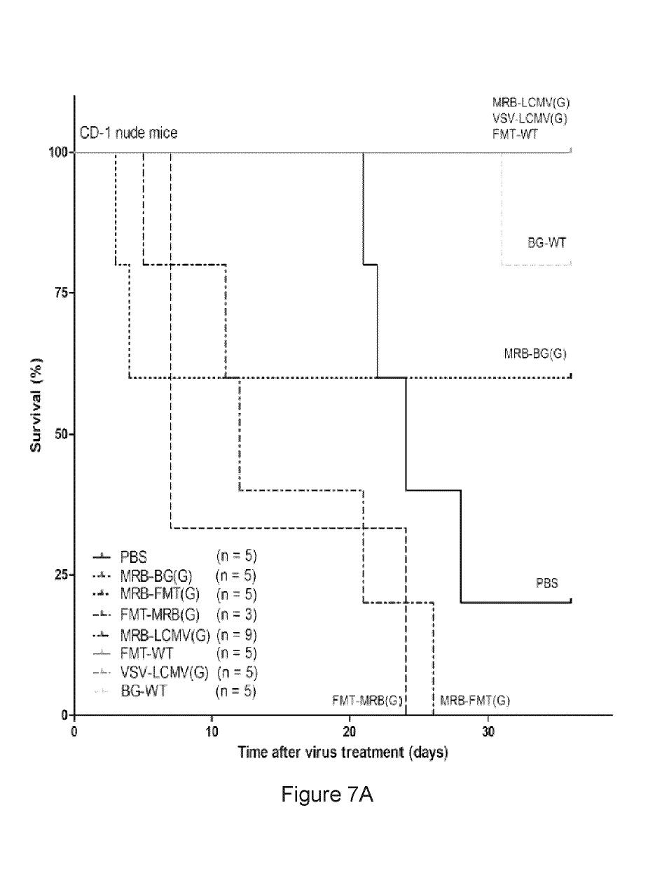

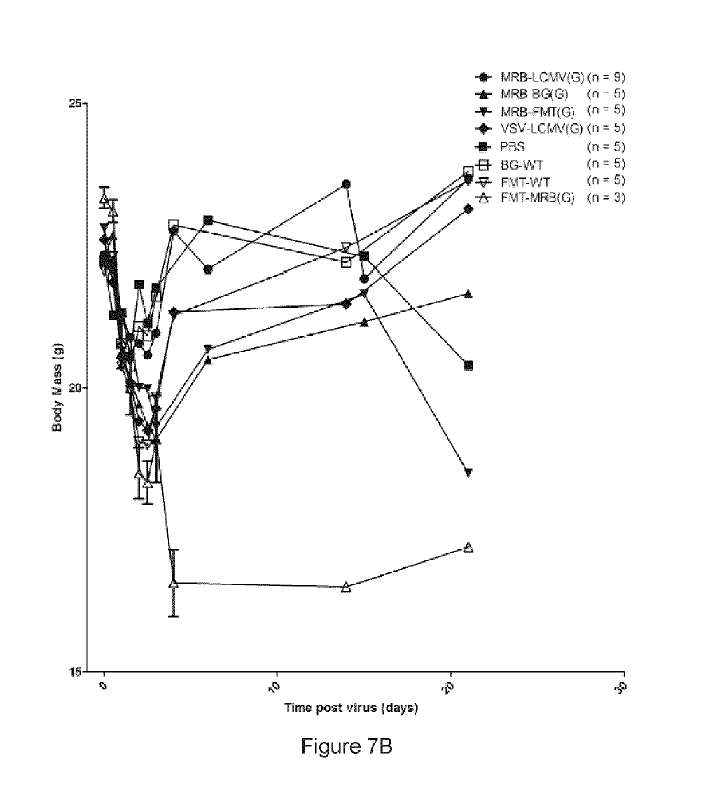

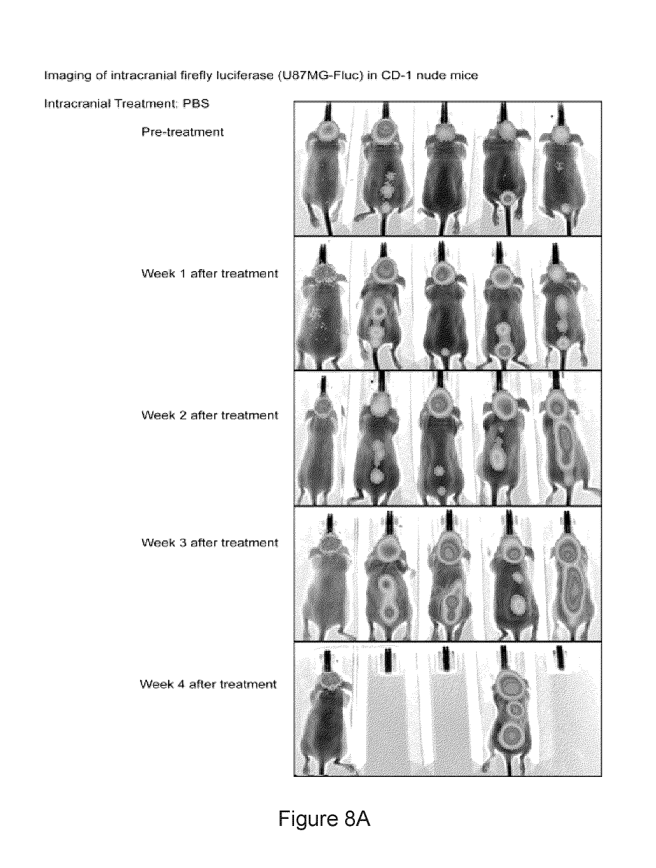

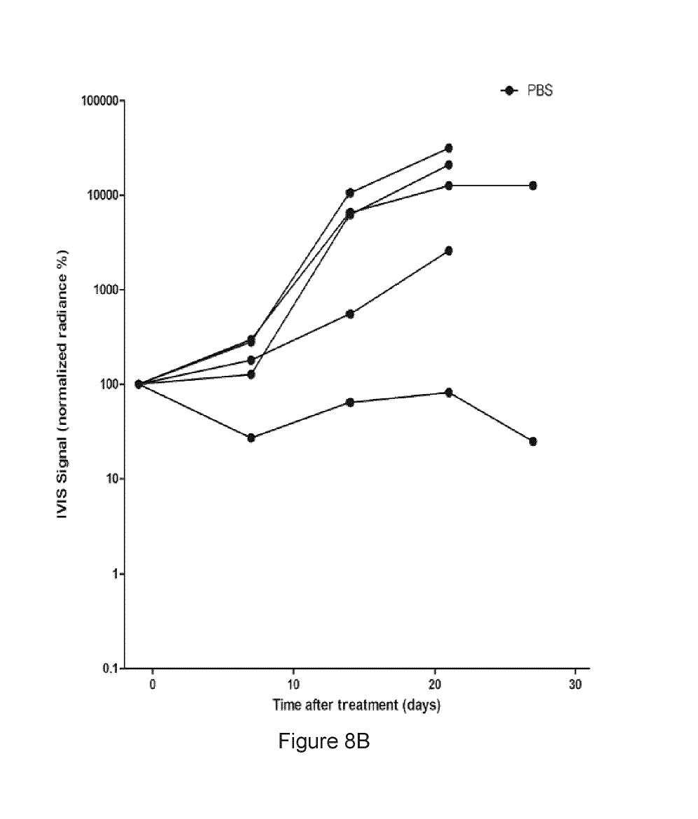

FIG. 7A is a graph illustrating in vivo efficacy of maraba chimeras according to the present disclosure versus control viruses. The graph shows Kaplan Meir survival plots of CD-1 nude mice with U87MG tumors post treatment. Animals treated with FMT-MRB(G) and MRB-FMT(G) survived more than 20 days but less than 30 days post IC injection. Animals treated with PBS survived to approximately 30 days post IC injection before succumbing to their tumors. Animals treated with MRB-BGG showed over 50% survival at 30 days post IC injection. Treatment with wild-type BG showed over 75% survival at 30 days post IC injection. Treatment with MRB-LCMV(G), VSV-LCMV(G) and FMT-WT showed 100% survival out to 30 days post IC injection. FIG. 7B is a graph showing the weight variation of the animals of FIG. 7A. All animals showed an initial drop in body mass 3-5 days after treatment. In animals treated with an IC injection of wild type FMT, BG or chimeras MRB LCMVG or VSV LCMVG the drop in body mass was temporary and animals recovered initial body mass by 20 days following treatment. Animals treated with chimera MRB FMTG or FMT MRBG or PBS controls did not show any recovery of body mass in the same time period. Detailed results are illustrated in FIGS. 8-15.

FIG. 8A is an IVIS image of U87MG tumours illustrating in vivo efficacy of PBS control in a human U87MG xenograft model. The image shows tumours pre and post (1 week, 2 weeks, 3 weeks, 4 weeks) treatment. FIG. 8B is a flux plot illustrating a significant increase in tumour burden over time in untreated control animals.

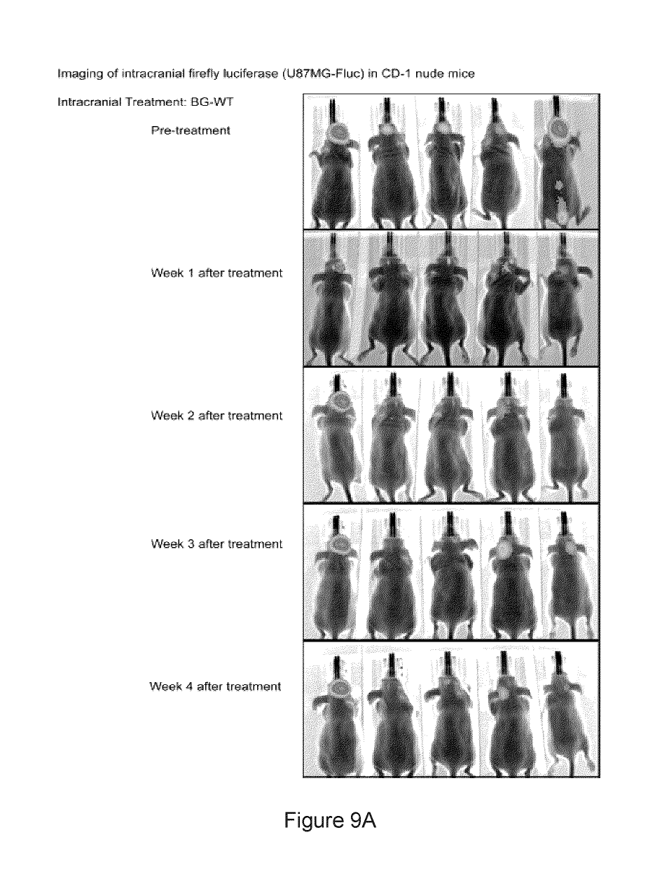

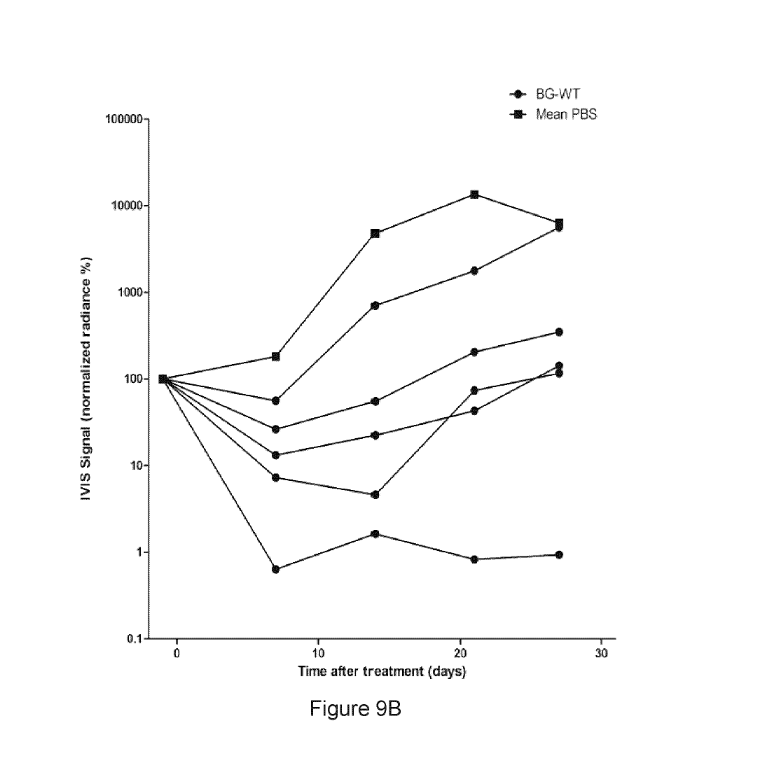

FIG. 9A is an IVIS image of U87MG tumours illustrating in vivo efficacy of BG wild type (BG-WT) virus treatment in a human U87MG xenograft model. The image shows U87MG tumours post BG (1 week, 2 weeks, 3 weeks, 4 weeks) treatment (1 dose 1e7 pfu: IC). FIG. 9B is a flux plot illustrating an initial moderate tumour regression in response to IC dose (1e7 pfu) of BG followed by a recurrence in tumour burden.

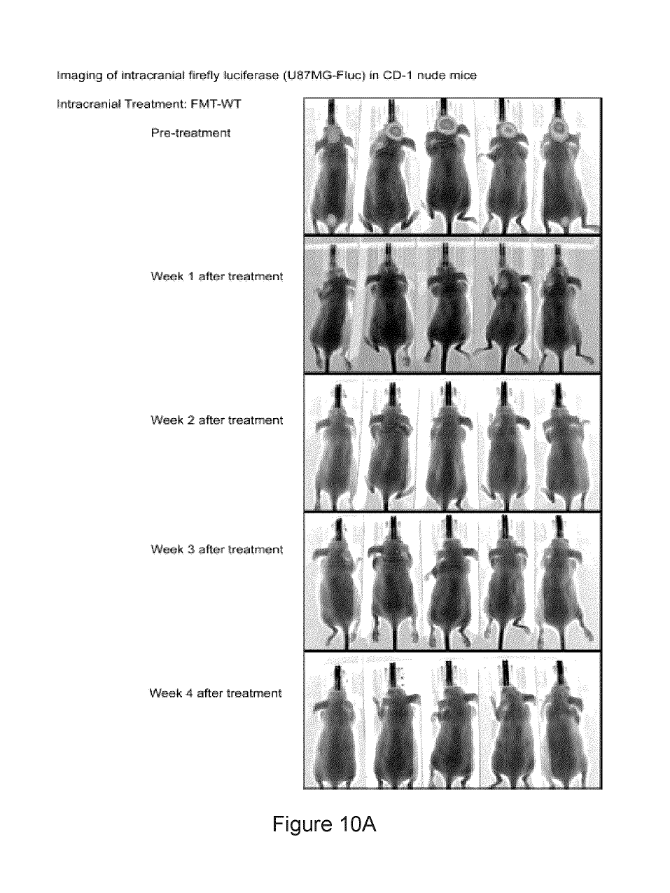

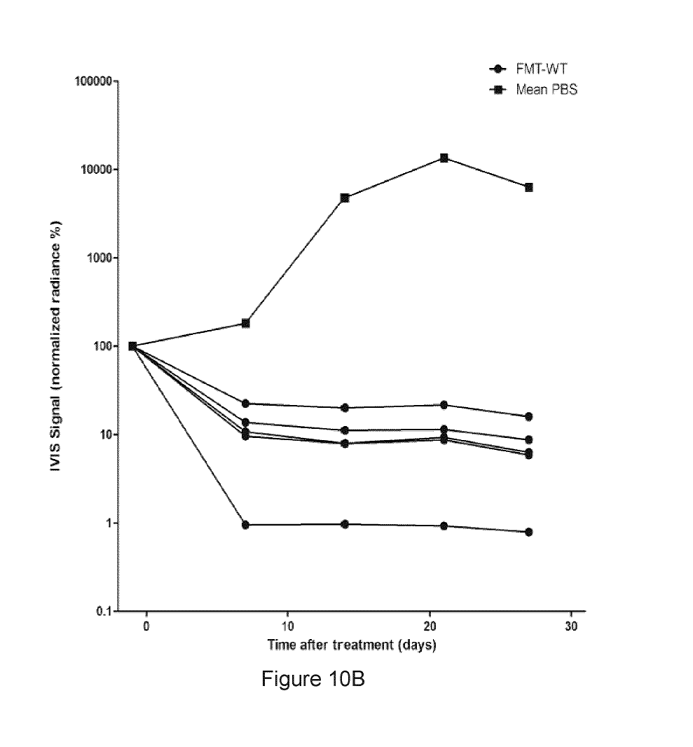

FIG. 10A is an IVIS image of U87MG tumours illustrating in vivo efficacy of FMT wild type (FMT-WT) virus treatment in a human U87MG xenograft model. The image shows U87MG tumours post FMT-WT (1 week, 2 weeks, 3 weeks, 4 weeks) treatment (1 dose 1e7 pfu: IC). FIG. 10B is a flux plot demonstrating a significant tumour regression in response to IC dose (1e7 pfu) of FMT-WT.

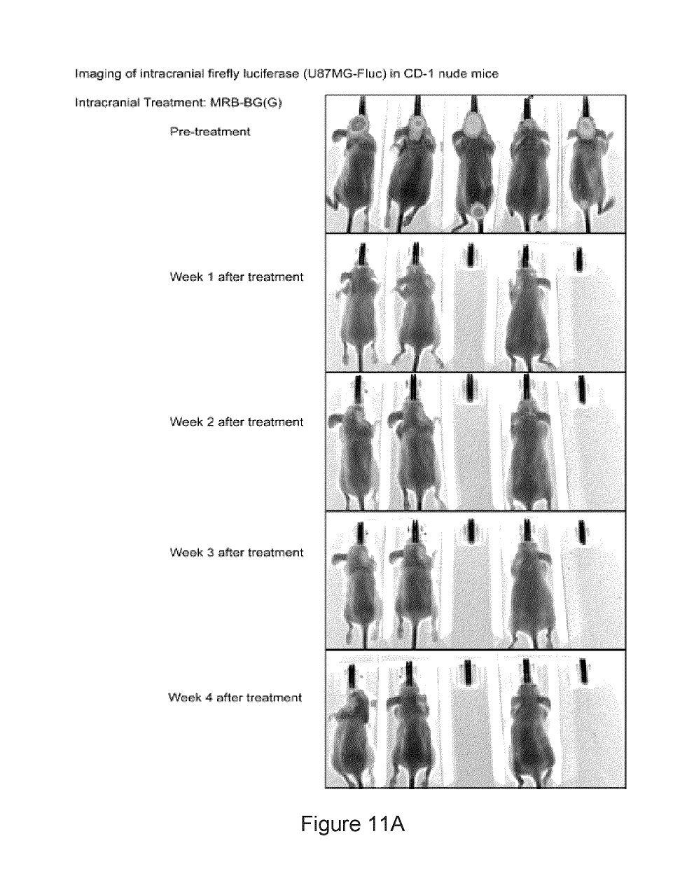

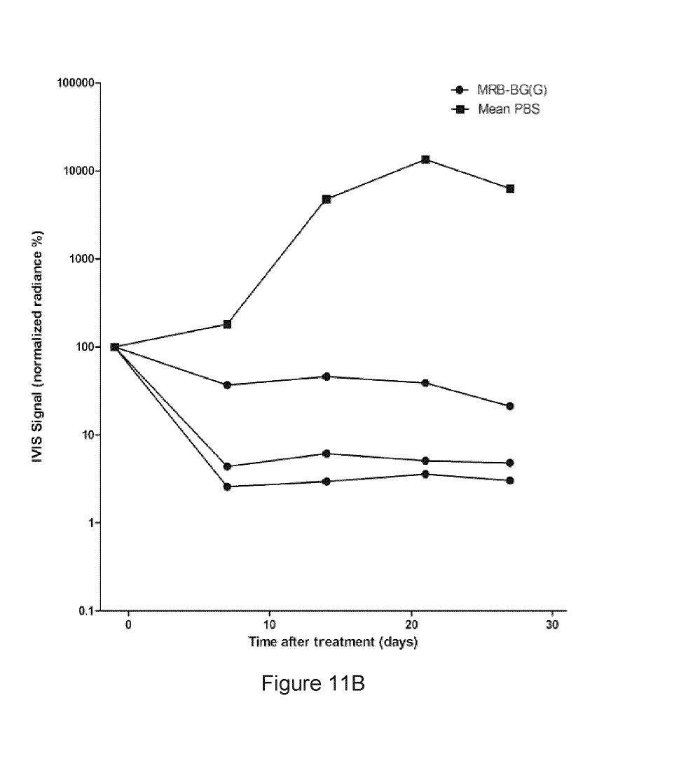

FIG. 11A is an IVIS image of U87MG tumours illustrating in vivo efficacy of MRB BG(G) treatment in a human U87MG xenograft model. The image shows U87MG tumours post MRB BG(G) (1 week, 2 weeks, 3 weeks, 4 weeks) treatment (1 dose 1e7 pfu: IC). FIG. 11B is a flux plot illustrating moderate tumour regression in response to IC dose (1e7 pfu) of MRB BGG.

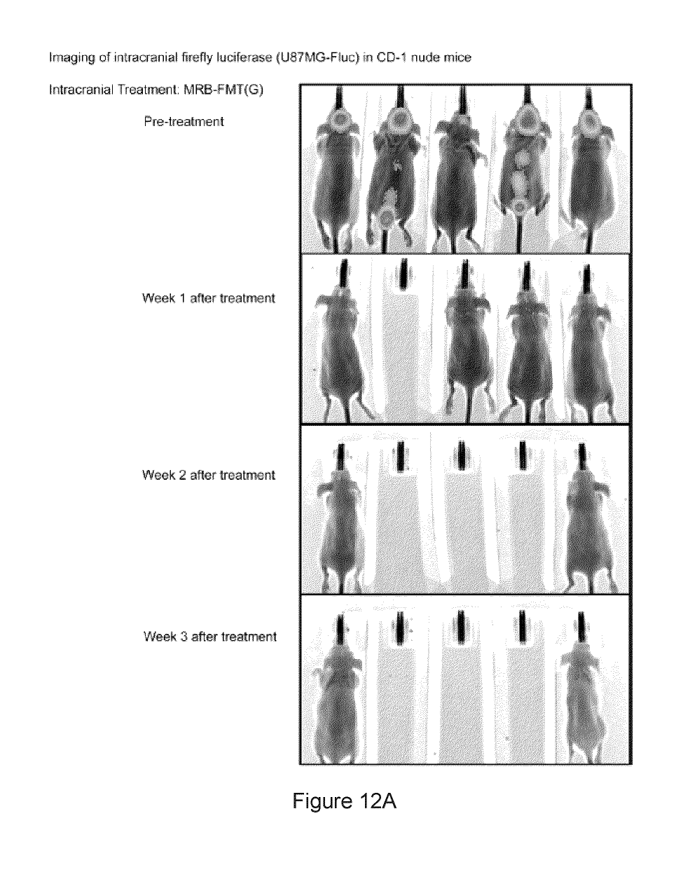

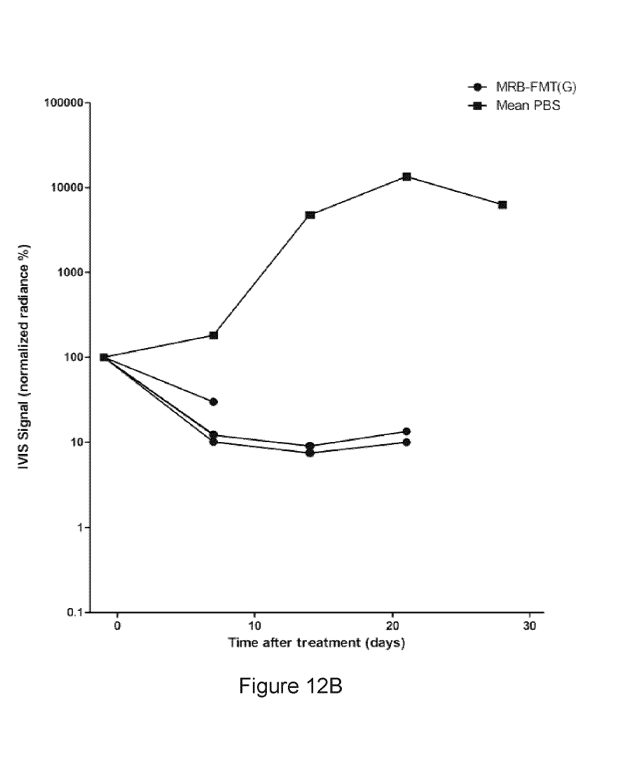

FIG. 12A is an IVIS image of U87MG tumours illustrating in vivo efficacy of MRB FMT(G) treatment in a human U87MG xenograft model. The image shows U87MG tumours post MRB FMT(G) (1 weeks, 2 weeks, 3 weeks) treatment (1 dose 1e7 pfu: IC). FIG. 12B is a flux plot demonstrating a significant tumour regression in response to IC dose (1e7 pfu: IC) of MRB FMT G. However, all animals succumbed to neurotoxic effects of MRB FMT(G) treatment prior to 4 weeks post treatment.

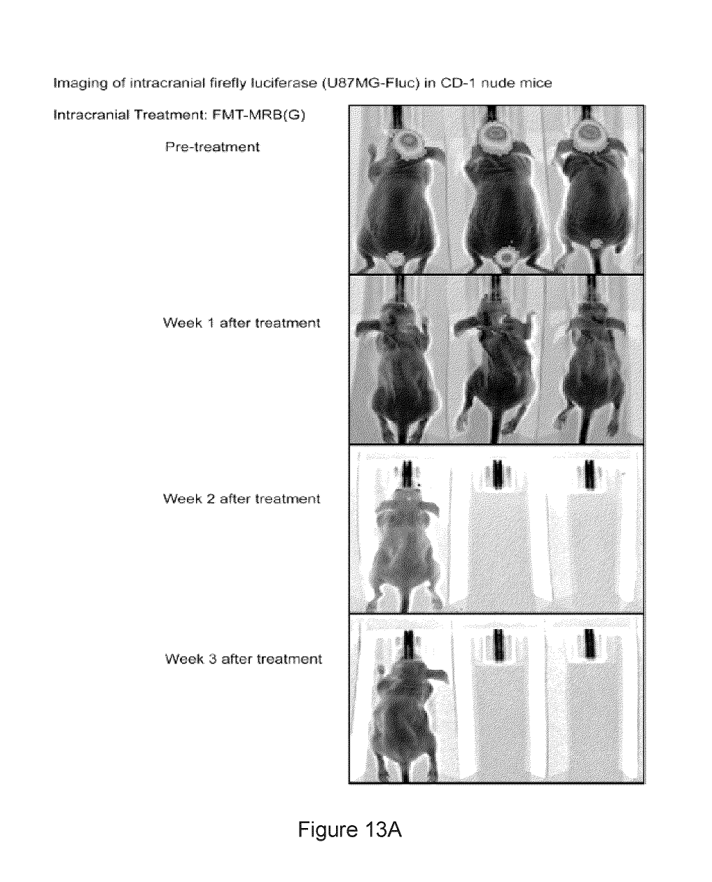

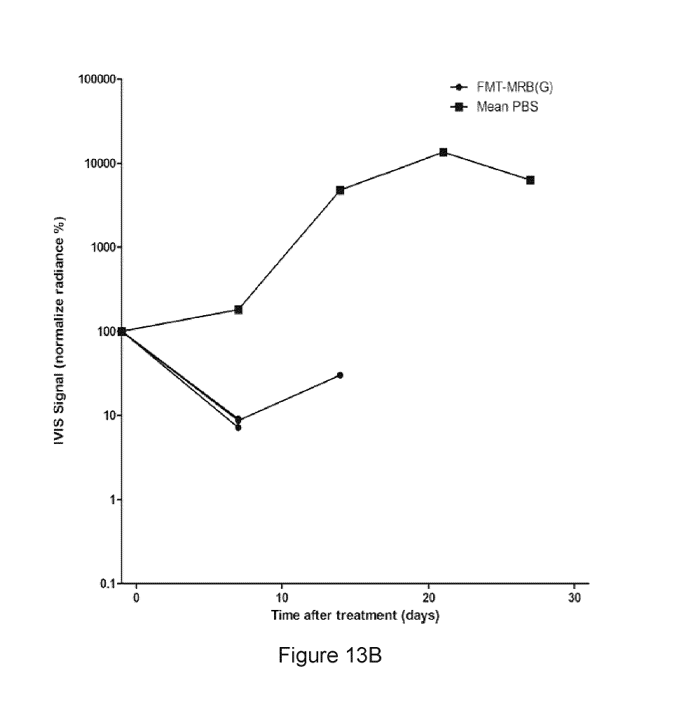

FIG. 13A is an IVIS image of U87MG tumours illustrating in vivo efficacy of FMT MRB(G) treatment in a human U87MG xenograft model. The image shows U87MG tumours post FMT MRB(G) (1 week, 2 weeks, 3 weeks) treatment (1 dose 1e7 pfu: IC). FIG. 13B is a flux plot illustrating a significant tumour regression in response to IC dose (1e7 pfu) of FMT MRB(G). However, all animals succumbed to neurotoxic effects of FMT MRB G treatment prior to 4 weeks post treatment.

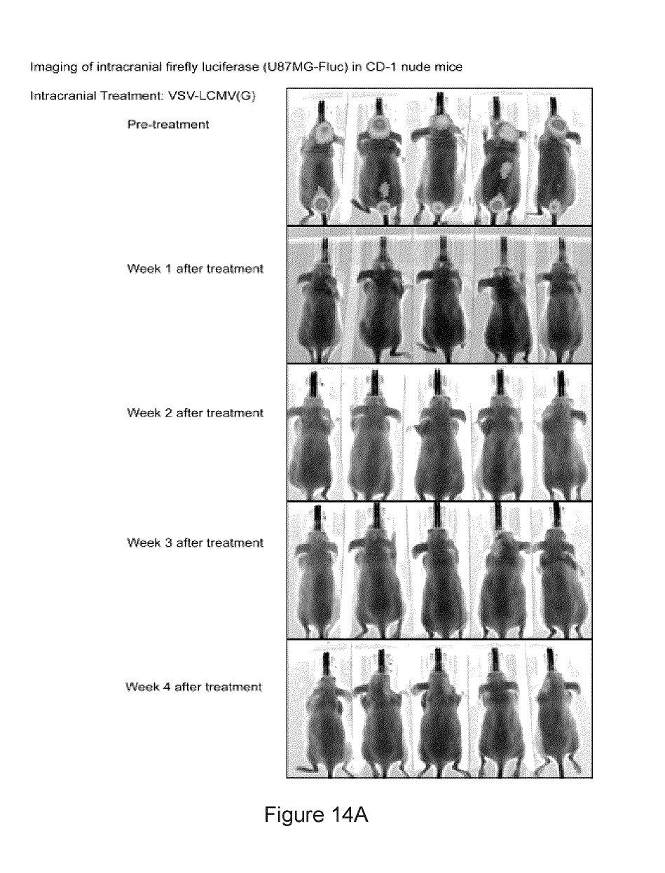

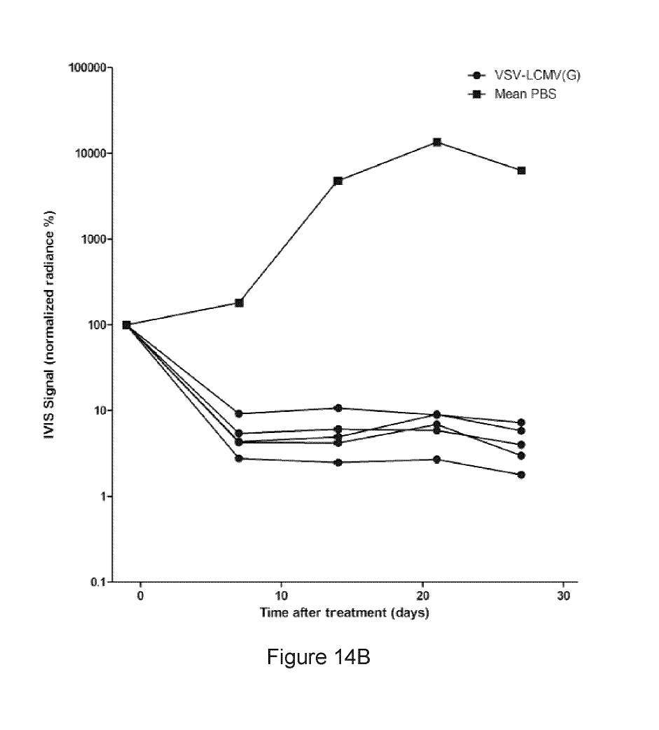

FIG. 14A is an IVIS image of U87MG tumours illustrating in vivo efficacy of VSV-LCMV(G) treatment in a human U87MG xenograft model. The image shows U87MG tumours post VSV LCMV G (1 week, 2 weeks, 3 weeks, 4 weeks) treatment (1 dose 1e7 pfu: IC). FIG. 14B is a flux plot illustrating a significant tumour regression in response to IC dose (1e7 pfu) of VSV-LCMV(G).

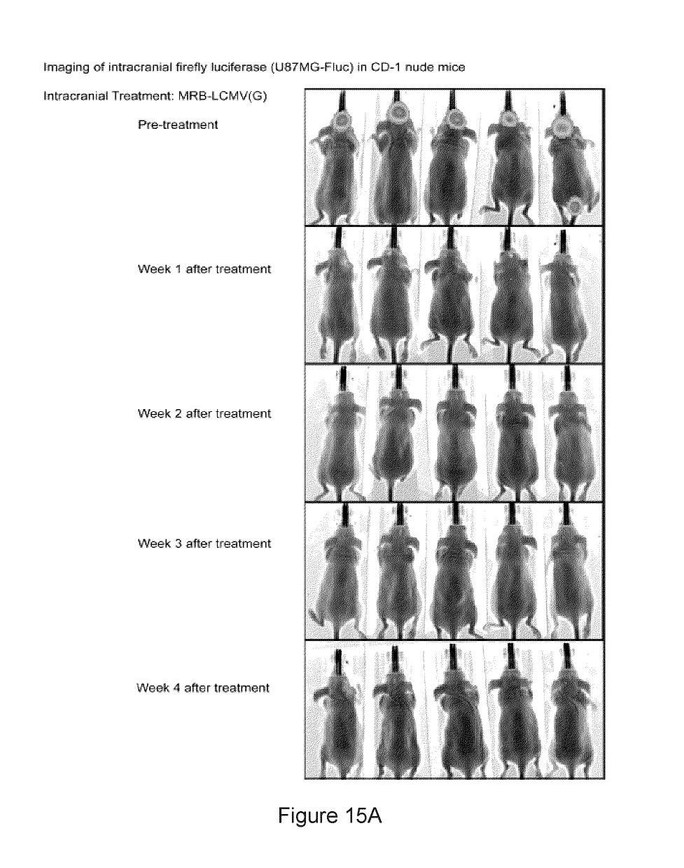

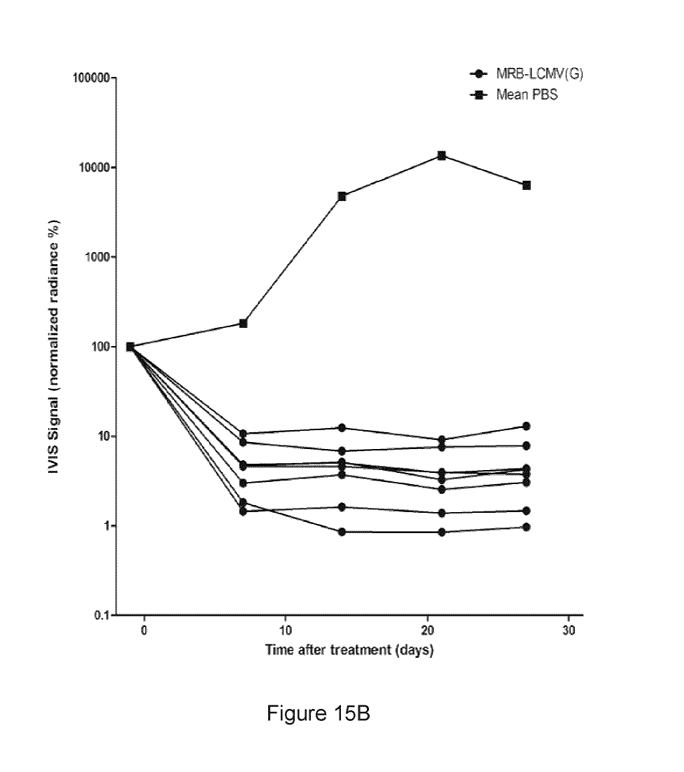

FIG. 15A is an IVIS image of U87MG tumours illustrating in vivo efficacy of MRB LCMV(G) treatment in a human U87MG xenograft model. The image shows U87MG tumours post MRB LCMV G (1 week, 2 weeks, 3 weeks, 4 weeks) treatment (1 dose 1e7 pfu: IC). FIG. 15B is a flux plot illustrating a significant tumour regression in response to IC dose (1e7 pfu) of MRB LCMV(G).

### Example 7

### Neutralizing Antibody Responses to Maraba Chimera Viruses

Assays to quantify the presence of neutralizing antibodies to indicated viruses were performed as previously described (Propagation, Purification, and In Vivo Testing of Oncolytic Vesicular Stomatitis Virus Strains, J-S Diallo et al., Oncolytic Viruses: Methods and Protocols, Methods in Molecular Biology Vol 797 (2012)).

Briefly, on day 0, 50 μL of saphenous vein blood from 6-8 week old female Balb/c mice was collected into heparin coated tubes, centrifuged and serum removed. Subsequently, three animals per group were injected intravenously by tail vein injection with 1e7 pfu of the indicated virus. Mice were again bled on day 7, then injected in the same manner as on day 0. Mice were bled a final time on day 14 by terminal cardiac puncture. Serum from each animal, from each of the three time points (day 0, 7, 14) was serially diluted at 1:2 across a 96 well plate, starting with an initial dilution of 1/50. Each serum-containing well was incubated with 2.5e4 pfu/well of the injected virus for one hour, giving an initial serum dilution of 1/100. The serum and virus mixture was then added to 96 well plates seeded the day before with 1.25e4 Vero cells/well. Two days later, monolayers were assessed by microscopy for evidence of cytopathic effect (CPE). The lowest dilution at which 50 percent CPE was evident determined the neutralizing antibody titer for a particular sample.

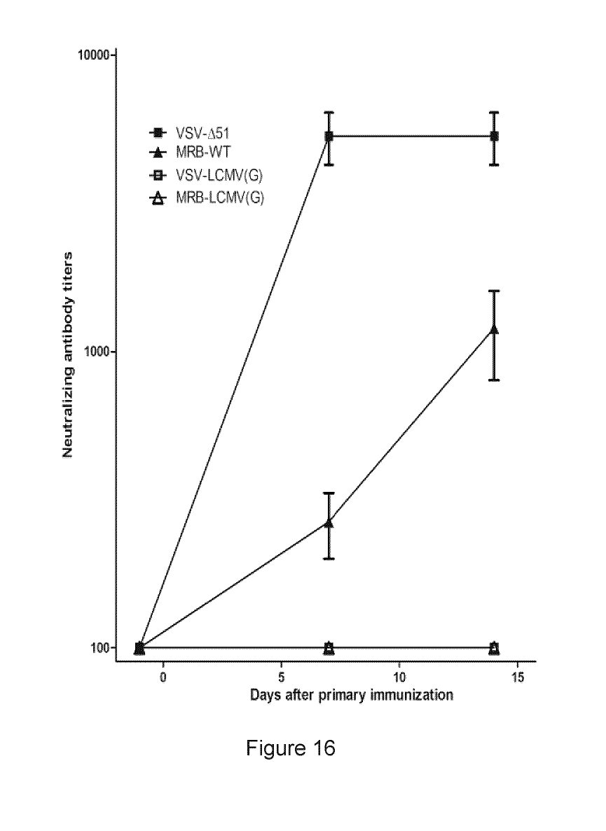

FIG. 16 is a graph illustrating the neutralizing antibody titres in Balb/C mice treated with attenuated VSV (VSV-Δ51) or wild type Maraba virus (MRB-WT) versus VSV-LCMV(G) or Maraba-LCMV(G) chimera viruses. The wild type MRB and VSV Δ51 (attenuated) induced significant neutralizing antibody titres while the corresponding chimeras VSV-LCMV(G) and MRB-LCMV(G) did not induce neutralizing antibody response. Reciprocal challenges of serum derived from day 14 mice were also performed. Serum collected from each of wild type MRB, VSV Δ51 (attenuated), VSV-LCMV(G), MRB-LCMV(G) was challenged with MRB-LCMV(G), VSV-LCMV(G), VSV-Δ51 (attenuated) and wild type MRB, respectively. In all cases a neutralizing antibody response was not evident.

### Example 8

### Chimera Virus Titres in Production Cells

To manufacture the indicated viruses, each were inoculated into forty 15 cm plastic tissue culture plates with subconfluent monolayers of Vero cells at a multiplicity of infection of 0.01. Twenty hours later, media was collected and virus was purified and titred as per Diallo et al 2012. Yield was calculated and each LCMV(G) chimera was compared to its parent wildtype. When compared to its parental strain, the MRB-LCMV(G) virus yielded over 2-fold more virus than VSV-LCMV(G (titre ratio VSV-LCMVG to wild type VSV is 0.028; in comparison, the titre ratio MRB-LCMV(G) to wild type MRB is 0.067).

In the preceding description, for purposes of explanation, numerous details are set forth in order to provide a thorough understanding of the examples. However, it will be apparent to one skilled in the art that these specific details are not required. The above-described examples are intended to be exemplary only. Alterations, modifications and variations can be effected to the particular embodiments by those of skill in the art without departing from the scope, which is defined solely by the claims appended hereto.

1. An isolated viral particle having a genome comprising open reading frames that encode:
a protein having a sequence comprising SEQ ID NO: 1, or a variant thereof;
a protein having a sequence comprising SEQ ID NO: 2, or a variant thereof;
a protein having a sequence comprising SEQ ID NO: 3, or a variant thereof;
a protein having a sequence comprising SEQ ID NO: 4 or 5, or a variant thereof; and
a protein having a sequence comprising SEQ ID NO: 6, 7 or 8.

2. The isolated viral particle according to claim 1 wherein the variant of a reference protein is a protein having a sequence which is at least 75%, at least 80%, at least 85%, at least 90%, or at least 95% identical to the sequence of the reference protein, and the variant protein maintains the same biological function as the reference protein.

3. The isolated viral particle according to claim 1, wherein the genome comprises an open reading frame that encodes a protein having a sequence comprising SEQ ID NO: 6.

4. The isolated viral particle according to claim 1, wherein the genome comprises an open reading frame that encodes a protein having a sequence comprising SEQ ID NO: 7.

5. The isolated viral particle according to claim 1, wherein the genome comprises an open reading frame that encodes a protein having a sequence comprising SEQ ID NO: 8.

6. The isolated viral particle according to claim 1, wherein the viral genome comprises open reading frames that encode:
a protein having a sequence comprising SEQ ID NO: 1;
a protein having a sequence comprising SEQ ID NO: 2;
a protein having a sequence comprising SEQ ID NO: 3;
a protein having a sequence comprising SEQ ID NO: 5; and
a protein having a sequence comprising SEQ ID NO: 7.

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. A method for treating cancer comprising administering an isolated viral particle according to claim 1 to a patient having cancer.

34. The method according to claim 33 wherein the cancer is a brain cancer.

35. The method according to claim 34 wherein the brain cancer is a glioblastoma.

36. The method according to claim 33 wherein the isolated viral particle is administered to the patient directly.

37. The method according to claim 36 wherein the isolated viral particle is administered directly to the central nervous system, outside the blood/brain barrier, inside the blood/brain barrier, or any combination thereof.

38. The method according to claim 37 wherein the isolated viral particle is administered to the patient intrathecally, intravenously, via intracranial injection, or any combination thereof simultaneously or sequentially.

39. The method according to claim 33 wherein a cell is infected with the isolated viral particle and the infected cell is administered to the patient.

40. The method according to claim 39 wherein the infected cell is administered directly to the central nervous system, outside the blood/brain barrier, inside the blood/brain barrier, or any combination thereof.

41. The method according to claim 39 wherein the infected cell is administered to the patient intrathecally, intravenously, via intracranial injection, or any combination thereof simultaneously or sequentially.

42. (canceled)

43. (canceled)

44. (canceled)

45. (canceled)

46. (canceled)

47. (canceled)

48. (canceled)

49. A kit for the treatment of cancer in a patient, the kit comprising:
the isolated viral particle according to claim 1; and
instructions for administration of the isolated viral particle to the patient.

50. The kit according to claim 49, wherein the cancer is a brain cancer.

51. The kit according to claim 49, wherein the brain cancer is a glioblastoma.

52. The kit according to claim 49 wherein the isolated viral particle is formulated for direct delivery to the central nervous system, outside the blood/brain barrier, inside the blood/brain barrier, or any combination thereof.

53. The kit according to claim 52 wherein the isolated viral particle is formulated for administration via intrathecal injection, intravenous injection, intracranial injection, or any combination thereof simultaneously or sequentially.

54. The kit according to claim 59 wherein the isolated viral particle is formulated for infection of a cell and the cell is for delivery to the central nervous system, outside the blood/brain barrier, inside the blood/brain barrier, or any combination thereof.

55. The kit according to claim 54 wherein the cell is for administration via intrathecal injection, intravenous injection, intracranial injection, or any combination thereof simultaneously or sequentially.
